(19) United States
(12) Patent Application Publication
(10) Pub. No.: US 2018/0110877 A1
(43) Pub. Date: Apr. 26, 2018

(54) DUAL AAV VECTOR SYSTEM FOR CRISPR/Cas9 MEDIATED CORRECTION OF HUMAN DISEASE

(71) Applicant: The Trustees of the University of Pennsylvania, Philadelphia, PA (US)

(72) Inventors: James M. Wilson, Philadelphia, PA (US); Lili Wang, Phoenixville, PA (US); Yang Yang, Chengdu (CN)

(21) Appl. No.: 15/566,150

(22) PCT Filed: Apr. 26, 2016
(86) PCT No.: PCT/US2016/029330

§ 371 Date: Oct. 12, 2017

Related U.S. Application Data
(60) Provisional application No. 62/153,470, filed on Apr. 27, 2015.
(60) Provisional application No. 62/183,825, filed on Jun. 24, 2015.
(60) Provisional application No. 62/254,225, filed on Nov. 12, 2015.
(60) Provisional application No. 62/287,511, filed on Jan. 27, 2016.

(51) Int. Cl. A61K 48/00; C12N 15/11; C12N 15/113; C12N 15/86; C12N 15/90; A61P 1/16; C12N 9/22

(52) U.S. Cl. CPC A61K 48/0058; C12N 15/111; C12N 15/113; C12N 15/86; C12N 2830/008; A61P 1/16; C12N 9/22; C12N 2310/20; C12N 2750/14143; C12N 15/907

(57) ABSTRACT

A dual vector expression system is provided for delivery of CRISPR/Cas9, sgRNA and donor templates for supplying corrected genes or gene sequences to neonatal subjects. An AAV8 system for delivery to proliferating cells such as hepatocytes is described, as are other dual AAV vector systems for targeting to other cells.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “UPN_15_7506PCT_ST25.txt”.

### STATEMENT REGARDING FEDERALLY SPONSORED RESEARCH OR DEVELOPMENT

This invention was made with government support under a grant from the National Institutes of Health, NICHD P01-HD057247. The US government has certain rights in this invention.

### BACKGROUND OF THE INVENTION

Early intervention and therapy is crucial in many inherited diseases. Various types of therapies have been described in the literature. One technique which has been described as having potential in correction of diseases associated with a genetic mutation or a specific phenotype is the Clustered Regularly Interspaced Short Palindromic Repeats (CRISPR) and CRISPR-associated protein (Cas9) system. CRISPR-Cas was derived from an adaptive immune response defense mechanism used by archaea and bacteria for the degradation of foreign genetic material [Van der Oost, J., et al. 2014. Nat. Rev. Microbiol. 7: 479-492; Hsu, P., et al. 2014. Development and applications of CRISPR-Cas9 for genome editing. Cell 157: 1262-1278]. This mechanism can be repurposed for other functions, including genomic engineering for mammalian systems, such as gene knockout (KO) [Cong, L., et al. 2013. Multiplex genome engineering using CRISPR/Cas systems. Science 339: 819-823; Mali, P., et al. 2013. RNA-guided human genome engineering via Cas9. Science 339: 823-826; Ran, F. A., et al. 2013. Genome engineering using the CRISPR-Cas9 system. Nat. Protoc. 8: 2281-2308; Shalem, O., et al. 2014. Genome-scale CRISPR-Cas9 knockout screening in human cells. Science 343: 84-87]. The CRISPR Type II system is currently the most commonly used RNA-guided endonuclease technology for genome engineering. There are two distinct components to this system: (1) a guide RNA and (2) an endonuclease, in this case the CRISPR associated (Cas) nuclease, Cas9. The guide RNA is a combination of the endogenous bacterial crRNA (CRISPR RNA) and tracrRNA (transactivating crRNA) into a single chimeric guide RNA (gRNA) transcript. The gRNA combines the targeting specificity of crRNA with the scaffolding properties of tracrRNA into a single transcript. When the gRNA and the Cas9 are expressed in the cell, the genomic target sequence can be modified or permanently disrupted.

Adeno-associated viruses have been described as being useful vectors for gene therapy. Such uses include those involving the CRISPR-Cas system. See, e.g., Yin et al, “Biotechnology, 32: 551-3 (2014) and “In vivo interrogation of gene function in the mammalian brain using CRISPR-Cas”, Nature Biotechnology, 33: 102-6 (2015).

As stated above, some inherited disorders require very early intervention, i.e., often within hours or days, in order to avoid infant mortality or significant morbidity. However, AAV vectors have been described as being unstable and therefore ineffective when delivered to neonates or infants; this has been attributed to the rapid proliferation of the liver in this stage of life, resulting in loss of the AAV vector by the proliferating cells and/or dilution of AAV-containing cells resulting in inadequate therapeutic outcomes.

What are needed are effective compositions and techniques for treating genetic disorders, and particularly those associated with neonate or infant mortality or significant morbidity in children and adults.

### SUMMARY OF THE INVENTION

The present invention provides a CRISPR-Cas system delivered via dual AAV vectors to newborn and infant subjects for targeted treatment of a genetic disorder. Advantageously, the system uses the rapid proliferation of the cells in the neonatal stage to populate tissues with the cells corrected by the AAV-mediated treatment while simultaneously diluting the AAV-mediated Cas elements. However, the method is also useful for treatment in older children and in adults for treatment of a variety of disorders, e.g., through targeting of proliferating, progenitor and/or stem cells in developing and adult tissues.

In one aspect, the invention provides a dual vector system for treating disorders, wherein the system comprises: (a) a gene editing vector comprising a Cas9 gene under control of regulatory sequences which direct its expression in a target cell (e.g., a hepatocyte) comprising a targeted gene which has one or more mutations resulting in a disease or disorder (e.g., a liver metabolic disorder); and (b) a targeting vector comprising one or more of sgRNAs and donor template, wherein the sgRNA comprises at least 20 nucleotides which specifically bind to a selected site in the targeted genes and is 5′ to a protospacer-adjacent motif (PAM) which is specifically recognized by the Cas9, and wherein the donor template comprises nucleic acid sequences which replaces at least one of the mutations in the targeted gene; wherein the ratio of gene editing vector of (a) to the vector containing template (b) is such that (b) is in excess of (a). In one embodiment, the disorder is a metabolic disorder. In another embodiment, the disorder is a liver metabolic disorder. In one embodiment, the vectors used in this system are adeno-associated virus (AAV) vectors. In one example, both the gene editing AAV vector and the targeting AAV vector have the same capsid.

In one aspect, the invention provides a method of treating a liver metabolic disorder in neonates, comprising: co-administering to the subject (a) a gene editing AAV vector comprising a Cas9 gene under control of regulatory sequences which direct its expression in a hepatocyte comprising a targeted gene which has one or more mutations resulting in a liver metabolic disorder; and (b) an AAV targeting vector comprising sgRNA and donor template, wherein the sgRNA comprises at least 20 nucleotides which specifically bind to a selected site in the targeted genes, and wherein the donor template comprises nucleic acid sequences which replaces at least one of the mutations in the targeted gene and which is 5′ to a PAM which is specifically recognized by the Cas9; wherein the ratio of gene editing AAV vector of (a) to (b) is such that (b) is in excess of (a). In one example, the liver metabolic disorder is ornithine transcarbamylase deficiency. In another aspect, the invention provides use of these AAV vectors to treat a liver metabolic disorder, or for the preparation of a medicament for the treatment of a liver metabolic disorder.

In a further aspect, the method is useful for treating genetic diseases in non-liver tissues by correcting point mutations by gene editing. This method allows for a single gene editing vector that simultaneously targets to different sites in order to increase the efficiency of the disease correction. This gene editing vector is co-administered to a subject with Cas9, and allows correction point mutations, as well as small deletions and insertions in the targeted cells. Suitable target tissues may include, e.g., gut epithelium, lung epithelium, hepatocytes, retina (e.g., retina epithelia), muscle and central nervous system progenitor cells.

In yet another aspect, method is useful for treating genetic diseases by inserting an entire gene cassette upstream of the targeted intron in a site-specific manner and downstream of the natural regulatory elements. This method is advantageous because it is independent from the specific location of a mutation. An exon or a large deletion or insertion can also be corrected by this method if an insertion cassette is followed by a donor site and is targeted to a donor site of the defective exon, so that gene correction will be achieved at the level of spliced mRNA. Coding sequences, as well as splice donor and acceptor sites can be corrected by the methods described herein.

Still other aspects and advantages of the invention will be readily apparent from the following detailed description of the invention.

### BRIEF DESCRIPTION OF THE DRAWINGS

FIGS. 1A-1C illustrate the results of a study showing in vivo gene correction of the OTC locus in the spf^{ash} mouse liver by AAV.CRISPR-SaCas9. FIG. 1A is a schematic diagram of the mouse OTC locus showing the spf^{ash} mutation and three SaCas9 targets. spf^{ash} has a G to A mutation at the donor splice site at the end of exon 4 indicated on the top strand. The nucleotide sequence reflected is SEQ ID NO: 1. The three selected SaCas9-targeted genomic loci (20 bp each) are in lighter gray and underlined with the PAM sequences marked. The black line above exon 4 indicates the 1.8 kb OTC donor template. FIG. 1B is a cartoon showing the dual AAV vector system for liver-directed and SaCas9-mediated gene correction. The AAV8.sgRNA1.donor vector contains a 1.8 kb murine OTC donor template sequence as shown in FIG. 1A with the corresponding PAM sequence mutated. FIG. 1C is a flowchart showing key steps of AAV8.CRISPR-SaCas9-mediated gene correction in the neonatal OTC spf^{ash} model.

FIG. 2A-2F illustrate efficient restoration of OTC expression in the liver of spf^{ash} mice by AAV8.CRISPR-SaCas9-mediated gene correction. AAV8.SaCas9 (5×10¹⁰ GC/pup) and AAV8.sgRNA1.donor (5×10¹¹ GC/pup) were administered to postnatal day 2 (p2) spf^{ash} pups via the temporal vein. spf^{ash} mice were sacrificed at 3 (n=5) or 8 weeks (n=8) after treatment. Untargeted spf^{ash} mice received AAV8.SaCas9 (5×10¹⁰ GC/pup) and AAV8.control.donor (5×10¹¹ GC/pup) at p2, and livers were harvested 8 weeks post treatment (n=6). FIG. 2A provides the quantification of gene correction based on the percentage of area on liver sections expressing OTC by immunostaining. FIG. 2B illustrates immunofluorescence staining with antibodies against OTC on liver sections from spf^{ash} mice treated with the dual AAV vectors for CRISPR-SaCas9-mediated gene correction at 3 weeks and 8 weeks. Stained areas typically represent clusters of corrected hepatocytes. Untreated controls show livers from wild type (wt), spf^{ash} heterozygous (het), and spf^{ash} hemizygous mice. Scale bar, 100 μm. FIG. 2C illustrates random distribution of clusters of corrected hepatocytes along the portal-central axis shown by double immunostaining against OTC and glutamine synthetase (GS) as a pericentral marker (p, portal vein; c, central vein). Scale bars, 300 μm (upper panel) and 100 μm (lower panel). FIG. 2D shows groups of corrected hepatocytes expressing OTC shown by immunofluorescence on sections counterstained with fluorescein-labeled tomato lectin (Lycopersicon esculentum lectin, LEL) which outlines individual hepatocytes. Scale bar, 50 μm. FIG. 2E shows OTC enzyme activity in the liver lysate of spf^{ash} mice at 3 and 8 weeks following dual vector treatment. FIG. 2F shows quantification of OTC mRNA levels in the liver by RT-qPCR using primers spanning exons 4-5 to amplify wild-type OTC. Mean±SEM are shown. * P<0.05, ** P<0.01, **** P<0.0001, Dunnett's test.

FIGS. 3A-3E shows the time course of SaCas9 expression following neonatal vector administration and functional improvement following high protein diet challenge. FIG. 3A shows the results of immunostaining with antibodies against Flag on liver sections from an untreated mouse or treated spf^{ash} mice at 1, 3, or 8 weeks following neonatal injection of the dual AAV vectors for CRISPR-SaCas9-mediated gene correction. AAV8.SaCas9 (5×10¹⁰ GC/pup) and AAV8.sgRNA1.donor (5×10¹¹ GC/pup) were administered to p2 spf^{ash} pups via the temporal vein. Nuclear staining of Flag-tagged SaCas9 were abundant at 1 week (n=5) but dramatically reduced at 3 weeks (n=6) and became scarce at 8 weeks (n=7) after vector injection. Scale bar, 100 μm. FIG. 3B shows the results of quantification of SaCas9 mRNA levels in liver by RT-qPCR. Mean±SEM are shown. * P<0.05, ** P<0.01, *** P<0.001, **** P<0.0001, Dunnett's test. FIG. 3C provides quantification of SaCas9 vector genome in liver by QPCR. FIG. 3D shows plasma ammonia levels in control or dual AAV vector-treated spf^{ash} mice after a one-week course of high protein diet. Seven weeks following neonatal treatment with the dual AAV vectors, mice were given high protein diet for 7 days. Plasma ammonia levels were measured 7 days after the high protein diet. Plasma ammonia levels in WT mice (n=13) and AAV8.SaCas9+AAV8.sgRNA1.donor-treated spf^{ash} mice (n=13) were significantly lower than untreated spf^{ash} mice (n=16) after a 7-day high protein diet. Shaded quares indicate samples obtained from moribund untreated spf^{ash} mice 6 days after high protein diet; shaded triangle indicates sample obtained from a moribund spf^{ash} mouse treated with untargeted vector (AAV8.control.donor with no sgRNA1, n=10) 5 days after high protein diet. ** P<0.01, **** P<0.0001, Dunnett's test. FIG. 3E shows survival curves in control or dual AAV vector-treated spf^{ash} mice after a one-week course of high protein diet. Untreated spf^{ash} mice (n=20) or spf^{ash} mice treated with untargeted vectors (AAV8.control.donor, n=13) started to die 3 days after high protein diet. All WT (n=13) and AAV8.SaCas9+AAV8.sgRNA1.donor-treated mice (n=13) survived. * P<0.05, Mantel-Cox test.

FIGS. 4A and 4B show in vitro validation of OTC sgRNAs and donor template. FIG. 4A shows in vitro validation of sgRNAs targeted to OTC in MC57G mouse cell line by transient transfection followed by 4-day puromycin enrichment and SURVEYOR® nuclease assay. sgRNA1 showed the highest efficiency in inducing indels in the targeted loci and was therefore chosen for subsequent studies. Arrows denote SURVEYOR® nuclease cleaved fragments of the OTC PCR products. Results were replicated in 2 independent experiments.

FIG. 4B shows in vitro validation of OTC donor template. MC57G cells were transiently transfected with a plasmid co-expressing OTC sgRNA1, SaCas9, and an AgeI restriction site tagged OTC donor plasmid followed by 4-day puromycin enrichment. RFLP analysis was performed following AgeI digestion to detect HDR in vitro. Co-transfection of the AgeI-tagged OTC donor template with an SaCas9 plasmid without OTC sgRNA1 did not result in detectable HDR. Arrows denote AgeI-sensitive cleavage products resulting from HDR. Results were replicated in 2 independent experiments. Indel and HDR frequency were calculated based on band intensities [L. Wang et al, Hum Gene Ther, 23: 533-539 (2012)].

FIGS. 5A and 5B show vector dose optimization to improve in vivo gene correction. Postnatal day 2 spf^{ash} pups received temporal vein injection of 5×10¹⁰ GC AAV8.SaCas9 and either 5×10¹⁰ (n=5), 1×10¹¹ (n=3), or 5×10¹¹ (n=5) GC of AAV8.sgRNA1.donor vector. Liver samples were collected 3 weeks post vector treatment for analysis. FIG. 5A shows quantification of gene correction based on the percentage of area on liver sections expressing OTC by immunostaining. FIG. 5B shows quantification of OTC mRNA levels in the liver by RT-qPCR using primers spanning exons 4-5 to amplify wild-type OTC. Mean±SEM are shown. ** P<0.01, Dunnett's test.

FIGS. 6A and 6B show the time course of gene expression by Western analysis and HDR analysis by RFLP. FIG. 6A provides HDR analysis by RFLP. OTC target region was PCR amplified from the liver genomic DNA isolated from untreated WT and spf^{ash} mice or spf^{ash} mice treated with the dual AAV vectors. Untreated WT and spf^{ash} control samples were collected at 8 weeks of age; samples from the treated spf^{ash} mice were collected at 1, 3, and 8 weeks (n=3 animals per time point) following neonatal injection of the dual AAV8 vectors. Targeted animals received AAV8.SaCas9 (5×10¹⁰ GC/pup) and AAV8.sgRNA1.donor (5×10¹¹ GC/pup); untargeted animals received AAV8.SaCas9 (5×10¹⁰ GC/pup) and AAV8.control.donor (5×10¹¹ GC/pup). AgeI digestion was performed and estimated HDR percentages are shown. FIG. 6B shows western analysis. Liver lysates were prepared from untreated WT and spf^{ash} mice or spf^{ash} mice treated with the dual AAV vectors for detection of Flag-SaCas9 and OTC protein.

FIGS. 7A and 7B show the examination of liver toxicity in animals treated with AAV8.CRISPR-SaCas9 dual vectors. FIG. 7A shows results of histological analysis on livers harvested 3 and 8 weeks following the dual vector treatment. FIG. 7B shows liver transaminase levels in untreated spf^{ash} mice (n=9) or 8 weeks following dual vector treatment. Untargeted mice received 5×10¹⁰ GC AAV8.SaCas9 and 5×10¹¹ of AAV8.control.donor vectors (n=8), while gene-targeted mice received 5×10¹⁰ GC AAV8.SaCas9 and 5×10¹¹ GC of AAV8.sgRNA1.donor (n=7). Mean±SEM are shown. There were no statistically significant differences between groups, Dunnett's test.

FIG. 8A-8E illustrate gene targeting/correction in the liver of spf^{ash} mice treated as adults by AAV8.CRISPR-SaCas9 vectors. Adult spf^{ash} mice (8-10 weeks old) received an intravenous injection of AAV8.SaCas9 (1×10¹¹ GC) and AAV8.sgRNA1.donor (1×10¹² GC), or higher dose of AAV8.SaCas9 (1×10¹² GC) and AAV8.sgRNA1.donor (5×10¹² GC), or untargeted vectors at the equivalent doses. 8A. Survival curve of the low-dose cohorts: sgRNA1 (n=10) or untargeted vector at the same dose (n=5). 8B Immunofluorescence staining with antibodies against OTC on liver sections collected at 3 (low-dose, n=3) or 2 weeks (high-dose, n=3) after injection. Stained cells typically showed as single corrected hepatocytes. 8C. Isolated corrected hepatocytes expressing OTC shown by immunofluorescence on sections co-stained with fluorescein-labeled tomato lectin (LEL) which outlines individual hepatocytes. Scale bar, 50 μm. 8D. Change of urine orotic acid levels in adult spf^{ash} mice after treatment with high-dose gene targeting vectors (n=3 for untreated spf^{ash} and low-dose groups; n=2 for high-dose groups). 8E. Elevation of plasma NH₃ levels in adult spf^{ash} mice after treatment with high-dose gene targeting vectors (n=3 for each group). Mean±SEM are shown. *** P<0.001, **** P<0.0001, Dunnett's test.

FIGS. 9A and 9B illustrate examination of liver toxicity in adult animals treated with AAV8.CRISPR-SaCas9 dual vectors. FIG. 9A shows the histological analysis on livers harvested 3 weeks (low-dose) or 2 weeks (high-dose) following dual vector treatment. Scale bar, 100 μm. FIG. 9B shows liver transaminase levels in untreated spf^{ash} mice, or 3 weeks following low-dose dual vector treatment, or 2 weeks following high-dose dual vector treatment (n=3 for each group). Low-dose, untargeted mice received 1×10¹¹ GC AAV8.SaCas9 and 1×10¹² GC of AAV8.control.donor vectors, while low-dose, gene-targeted mice received 1×10¹¹ GC AAV8.SaCas9 and 1×10¹² GC of AAV8.sgRNA1.donor. High-dose, untargeted mice received 1×10¹² GC AAV8.SaCas9 and 5×10¹² GC of AAV8.control.donor vectors, while high-dose, gene-targeted mice received 1×10¹² GC AAV8.SaCas9 and 5×10¹² GC of AAV8.sgRNA1.donor. Mean±SEM are shown. Adult animals received high-dose, gene-targeted vectors showed a trend of elevated ALT and AST levels, although not statistically different when compared with other groups (Dunnett's test).

FIG. 10 is a cartoon showing the location of sgRNA1, sgRNA2 and sgRNA3 assessed for correction of MPSI as described in Example 2. The nucleotide sequence reflected is SEQ ID NO: 2.

FIG. 11 is a cartoon illustrating the dual AAV vector system for liver-directed and AsCpf1-mediated gene correction. The AAV8.sgRNA.donor vector contains a 1.8 kb donor template sequence with the corresponding PAM sequence mutated.

FIG. 12 is a cartoon illustrating the dual AAV vector system for liver-directed and LbCpf1-mediated gene correction. The AAV8.sgRNA.donor vector contains a 1.8 kb donor template sequence with the corresponding PAM sequence mutated.

FIG. 13A is a cartoon showing the AAV.sgRNA.PCSK9.ScCas9 plasmid. FIG. 13B illustrates the serum PCSK9 levels in a rhesus monkey following administration of AAV8.sgRNA.PCSK9.TBG-S1.SaCas9 vector (3×10¹³ GC/kg). Approximately ˜40% reduction at day 14 post vector administration is observed compared to day 0.

### DETAILED DESCRIPTION OF THE INVENTION

The present invention provides a dual vector system to express sgRNA and donor template RNA for precise in vivo gene correction of a disease, disorder or condition characterized by a genetic abnormality. This system is particularly well suited for delivery to hepatocytes during the neonatal stage and/or infancy, but may be used in the pre-natal stage, or in older children or adults for targeting other cell types. Examples of other cell types are proliferating, progenitor and/or stem cells in young and adult patients, and optionally, post-mitotic cells. Such cells may include, e.g., epithelial cells (gut, lung, retina, etc), central nervous system (CNS) progenitor cells, among others.

The term “proliferating cells” as used herein refers to cells which multiply or reproduce, as a result of cell growth and cell division. Cells may be naturally proliferating at a desired rate, e.g., epithelial cells, stem cells, blood cells, hepatocytes; in such embodiments, the invention takes advantage of the natural proliferation rate of the cells as described herein. In another embodiment, cells may be proliferating at an abnormal or undesirable rate, e.g., as in cancer cells, or the excessive hyperplastic cell growth associated with the occlusive vascular lesions of atherosclerosis, restenosis post-angioplasty, and graft atherosclerosis after coronary artery bypass. In this embodiment, the invention may either use the proliferation rate of these cells and/or make seek to alter the proliferation rate (e.g., by correcting a genetic abnormality which results in a high growth rate).

As used herein, the term “disorder” or “genetic disorder” is used throughout this specific to refer to any diseases, disorders, or condition associated with an insertion, change or deletion in the amino acid sequence of the wild-type protein. Unless otherwise specified such disorders include inherited and/or non-inherited genetic disorders, as well as diseases and conditions which may not manifest physical symptoms during infancy or childhood.

In general, a neonate in humans may refer to infants from birth to under about 28 days of age; and infants may include neonates and span up to about 1 year of age to up to 2 years of age. The term “young children” may span to up to about 11-12 years of age.

The dual CRISP-Cas vector system provided herein utilizes a combination of two different vector populations co-administered to a subject. These vectors may be formulated together or separately and delivered essentially simultaneously, preferably by the same route. The working examples below describe use of AAV vectors. While the following discussion focuses on AAV vectors, it will be understood that a different, partially or wholly integrating virus (e.g., another parvovirus or a lentivirus) may be used in the system in place of the gene editing vector and/or the vector carrying template.

In one example, the dual vector system comprises (a) a gene editing vector which comprises a gene for an editing enzyme under control of regulatory sequences which direct its expression in a target cell (e.g., a hepatocyte) comprising a targeted gene which has one or more mutations resulting in a disorder (e.g., a liver metabolic disease) and (b) a targeting vector comprising a sequence specifically recognized by the editing enzyme and donor template, wherein the donor template comprises nucleic acid sequences which replaces at least one of the mutations in the targeted gene.

In one embodiment, the gene editing vector comprises a Cas9 gene as the editing enzyme and the targeting vector comprises sgRNA which is at least 20 nucleotides in length which specifically bind to a selected site in the targeted genes and is 5′ to a protospacer-adjacent motif (PAM) which is specifically recognized by the Cas9. Typically, the PAM sequence to the corresponding sgRNA is mutated on the donor template. However, in another embodiment, the gene editing vector may contain a different Crispr.

“Cas9” (CRISPR associated protein 9) refers to family of RNA-guided DNA endonucleases which is characterized by two signature nuclease domains, RuvC (cleaves non-coding strand) and HNH (coding strand). Suitable bacterial sources of Cas9 include Staphylococcus aureus (SaCas9), Stapylococcus pyogenes (SpCas9), and Neisseria meningitides [KM Estelt et al, Nat Meth, 10: 1116-1121 (2013)]. The wild-type coding sequences may be utilized in the constructs described herein. Alternatively, these bacterial codons are optimized for expression in humans, e.g., using any of a variety of known human codon optimizing algorithms. Alternatively, these sequences may be produced synthetically, either in full or in part. In the examples below, the Staphylococcus aureus (SaCas9) and the Stapylococcus pyogenes (SpCas9) versions of cas9 were compared. SaCas9 has a shorter sequence. Other endonucleases with similar properties may optionally be substituted. See, e.g., the public CRISPR database (db) accessible at http://crispr.u-psud.fr/crispr.

In another embodiment, the CRISPR system selected may be Cpf1 (CRISPR from Prevotella and Francisella), which may be substituted for Class 2 CRISPR, type II Cas9-based system in the methods described herein. In contrast, Cpf1's preferred PAM is 5′-TTN; this contrasts with that of SpCas9 (5′-NGG) and SaCas9 (5′-NNGRRT; N=any nucleotide; R=adenine or guanine) in both genomic location and GC-content. While at least 16 Cpf1 nucleases, two humanized nucleases (AsCpf1 and LbCpf1) are particularly useful. See, http://www.addgene.org/69982/sequences/#depositor-full (AsCpf1 sequences; and http://www.addgene.org/69988/sequences/#depositor-full (LbCpf1 sequences), which are incorporated herein by reference. Further, Cpf1 does not require a tracrRNA; allowing use of shorter guide RNAs (about 42 nucleotides) as compared to Cas9. Plasmids may be obtained from Addgene, a public plasmid database.

While the system can be effective if the ratio of gene editing vector to template vector is about 1 to about 1, it is desirable for the template vector to be present in excess of the gene editing vector. In one embodiment, the ratio of editing vector (a) to targeting vector (b) is about 1:3 to about 1:100, or about 1:10. This ratio of gene editing enzyme (e.g., Cas9 or Cpf) to donor template may be maintained even if the enzyme is additionally or alternatively supplied by a source other than the AAV vector. Such embodiments are discussed in more detail below.

A variety of conventional vector elements may be used for delivery of the editing vector to the target cells and expression of the enzyme (Cas9 or Cpf1). However, for the dual vector system designed for treatment of metabolic disorders characterized by a mutation or phenotype in hepatocytes, the gene editing vector may designed such that the enzyme is expressed under the control of a liver-specific promoter. The illustrative plasmid and vector described herein uses the liver-specific promoter thyroxin binding globulin (TBG) or a novel shortened version of TBG, a variant termed herein TBG-S1, which is characterized by the sequence:

(SEQ ID NO: 3)
actcaaagttcaaaccttatcattttttgctttgttcctcttggccttgg
ttttgtacatcagctttgaaaataccatcccagggttaatgctggggtta
atttataactaagagtgctctagttttgcaatacaggacatgctataaaa
atggaaagatgttgctttctgagaga.

Alternatively, other liver-specific promoters may be used [see, e.g., The Liver Specific Gene Promoter Database, Cold Spring Harbor, http://rulai.schl.edu/LSPD, alpha 1 anti-trypsin (A1AT); human albumin Miyatake et al., J. Virol., 71:5124 32 (1997), humAlb; and hepatitis B virus core promoter, Sandig et al., Gene Ther., 3:1002 9 (1996)]. TTR minimal enhancer/promoter, alpha-antitrypsin promoter, LSP (845 nt). For a different target tissue (e.g., epithelial cells, CNS), a different tissue specific promoter may be selected. Examples of promoters specific for endothelial cells include, but are not limited to, endothelin-I (ET-I), Flt-I, FoxJ1 (that targets ciliated cells), and T3^{b} [H Aihara et al, FEBS Letters, Vol. 463 (Issues 1-2), p. 185-188 (10 Dec. 1999) (targeting intestinal epithelial cells), E-cadherin promoter (J. Behrens et al, Proc Natl Acad Sci USA, Vol. 88: 11495-11499 (December 1991)], CEA promoter. Examples of neuron-specific promoters include, e.g., synapsin I (SYN), calcium/calmodulin-dependent protein kinase III, tubulin alpha I, microtubulin-associated protein 1B (MAP1B), neuron-specific enolase (Andersen et al., Cell. Mol. Neurobiol, 13:503-15 (1993)), and platelet-derived growth factor beta chain promoters, neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), neuronal nuclei (NeuN), glial fibrillary acidic protein (GFAP), adenomatous polyposis coli (APC), and ionized calcium-binding adapter molecule 1 (Iba-1), minimal promoter for HB9 [S Pfaff, Neuron (1999) 23: 675-687; Nature Genetics (1999) 23: 71-75]. Non-tissue specific promoters can also be used. Optionally, such a promoter may be an enhancer (e.g., cytomegalovirus). Alternatively, other promoters, such as constitutive promoters, regulatable promoters [see, e.g., WO 2011/126808 and WO 2013/049493, incorporated by reference herein], or a promoter responsive to physiologic cues may be utilized in the vectors described herein. Alternatively, for a different target tissue, Optionally, if a regulatable system is selected, a third vector may be required in order to provide the regulatory function.

Among other conventional vector elements which may be used in the gene editing vector are expression enhancer elements. One desirable enhancer is the novel ABPS2 (2 repeats of shortened ABP enhancer element):

(SEQ ID NO: 4)
gttaatttttaaactgtttgctctggttaataatctcaggaggttaattt
ttaaactgtttgctctggttaataatctca.

However, other suitable enhancers may include, e.g., the alpha fetoprotein enhancer, the TTR minimal promoter/enhancer, LSP (TH-binding globulin promoter/alpha1-microglobulin/bikunin enhancer), amongst others. Yet other promoters and enhancers can be used to target liver and/or other tissues. Other suitable vector elements may also be included in this gene editing vector. However, the size of the enzyme (Cas9 or Cpf1) gene and packaging limitations of AAV does make it desirable to select truncated or shortened versions of such elements. Thus, while conventional polyA sequences may be selected including, e.g., SV40 and bovine growth hormone (bGH), shortened and/or synthetic polyAs may also be desired.

In addition to the gene editing vector, the dual AAV vector system utilizes a second type of vector which is an AAV targeting vector comprising sgRNA and donor template. Optionally, more than 1 sgRNA can be used to improve the rates of gene correction. The term “sgRNA” refers to a “single-guide RNA”. sgRNA has at least a 20 base sequence (or about 24-28 bases) for specific DNA binding (homologous to the target DNA). Transcription of sgRNAs should start precisely at its 5′ end. When targeting the template DNA strand, the base-pairing region of the sgRNA has the same sequence identity as the transcribed sequence. When targeting the nontemplate DNA strand, the base-pairing region of the sgRNA is the reverse-complement of the transcribed sequence. Optionally, the targeting vector may contain more than one sgRNA. The sgRNA is 5′ to a protospacer-adjacent motif (PAM) which is specifically recognized by the Cas9 (or Cpf1) enzyme. Typically, the sgRNA is “immediately” 5′ to the PAM sequence, i.e., there are no spacer or intervening sequences. Examples of sgRNA and PAM sequences designed for correcting a mutation in the OTC gene which causes OTC deficiency are illustrated below. More particularly, the following target sequences are designed to correct the G/A mutation associated with OTC deficiency in the position corresponding to nt 243 of wt OTC by inserting (or knocking-in) a fragment containing the correct sequence [see, e.g., Genbank entry D00230.2, for genomic DNA sequence and identification of introns and exons, http://www.ncbi.nlm.nih.gov/nuccore/-D00230.2].

In general, a PAM sequence for SaCas9 has an NNGRRT motif. Once a selected target sequence is selected, an sgRNA comprising the target and PAM sequence may be generated synthetically, or using conventional site-directed mutagenesis. In the examples below, illustrating correction of the ornithine transcarbamylase (OTC) gene, the target DNA is within intron 4, which is 3′ to the G/A mutation site. However, other suitable target sites may be selected for other mutations targeted for correction. See, e.g., http://omim.org/entry/311250.

TABLE 1
Allelic Variants (29 Selected Examples)

| Number | Phenotype | Mutation | dbSNP | ClinVar |
|---|---|---|---|---|
| .0001 | ORNITHINE | OTC, DEL | | RCV000011733 |
| | TRANSCARBAMYLASE | | | |
| | DEFICIENCY | | | |
| .0002 | ORNITHINE | OTC, ARG109GLN | rs68026851 | RCV000083434, RCV000011734 |
| | TRANSCARBAMYLASE | | | |
| | DEFICIENCY | | | |
| .0003 | ORNITHINE | OTC, ARG109TER | rs67960011 | RCV000083433, RCV000011735 |
| | TRANSCARBAMYLASE | | | |
| | DEFICIENCY | | | |
| .0004 | ORNITHINE | OTC, LEU111PRO | rs1800324 | RCV000011736 |
| | TRANSCARBAMYLASE | | | |
| | DEFICIENCY | | | |
| .0005 | ORNITHINE | OTC, GLN216GLU | rs72558423 | RCV000011737, RCV000083523 |
| | TRANSCARBAMYLASE | | | |
| | DEFICIENCY | | | |
| .0006 | ORNITHINE | OTC, GLU154TER | rs72556267 | RCV000011738, RCV000083446 |
| | TRANSCARBAMYLASE | | | |
| | DEFICIENCY | | | |
| .0007 | ORNITHINE | OTC, LEU45PRO | rs72554312 | RCV000083338, RCV000011739 |
| | TRANSCARBAMYLASE | | | |
| | DEFICIENCY | | | |
| .0008 | ORNITHINE | OTC, ARG26GLN | rs68031618 | RCV000011740, RCV000083565 |
| | TRANSCARBAMYLASE | | | |
| | DEFICIENCY | | | |
| .0009 | ORNITHINE | OTC, LYS46ARG | rs1800321 | RCV000079082, RCV000011741 |
| | TRANSCARBAMYLASE | | | |
| | POLYMORPHISM | | | |
| .0010 | ORNITHINE | OTC, ARG245TRP | | RCV000011742 |
| | TRANSCARBAMYLASE | | | |
| | DEFICIENCY | | | |
| .0011 | ORNITHINE | OTC, GT-GC, | rs72558431 | RCV000011743, RCV000083544 |
| | TRANSCARBAMYLASE | INTRON 7 | | |
| | DEFICIENCY | | | |
| .0012 | ORNITHINE | OTC, GTA-GTG, INTRON 7 | | RCV000011744 |
| | TRANSCARBAMYLASE | | | |
| | DEFICIENCY | | | |
| .0013 | ORNITHINE | OTC, IVS4, A-T, -2 | rs66556380 | RCV000083419, RCV000011745 |
| | TRANSCARBAMYLASE | | | |
| | DEFICIENCY | | | |
| .0014 | ORNITHINE | OTC, ARG277TRP | rs72558454 | RCV000083586, RCV000011746 |
| | TRANSCARBAMYLASE | | | |
| | DEFICIENCY | | | |
| .0015 | ORNITHINE | OTC, PRO225LEU | rs67120076 | RCV000083536, RCV000011747 |
| | TRANSCARBAMYLASE | | | |
| | DEFICIENCY | | | |
| .0016 | ORNITHINE | OTC, GLU87LYS | rs72554338 | RCV000011748, RCV000083376 |
| | TRANSCARBAMYLASE | | | |
| | DEFICIENCY | | | |
| .0017 | ORNITHINE | OTC, GLY50TER | rs67486158 | RCV000083346, RCV000011749 |
| | TRANSCARBAMYLASE | | | |
| | DEFICIENCY | | | |
| .0018 | ORNITHINE | OTC, GLY162ARG | rs66626662 | RCV000083456, RCV000011750 |
| | TRANSCARBAMYLASE | | | |
| | DEFICIENCY | | | |
| .0019 | ORNITHINE | OTC, 1-BP DEL, 403G | | RCV000011751 |
| | TRANSCARBAMYLASE | | | |
| | DEFICIENCY | | | |
| .0020 | ORNITHINE | OTC, IVS2, G-A, -1 | | RCV000011752 |
| | TRANSCARBAMYLASE | | | |
| | DEFICIENCY | | | |
| .0021 | ORNITHINE | OTC, GLY47GLU | rs72554331 | RCV000011753, RCV000083369 |
| | TRANSCARBAMYLASE | | | |
| | DEFICIENCY | | | |
| .0022 | ORNITHINE | OTC, ARG62THR | rs72554345 | RCV000083388, RCV000011754 |
| | TRANSCARBAMYLASE | | | |
| | DEFICIENCY | | | |
| .0023 | ORNITHINE | OTC, LEU272PHE | rs72558465 | RCV000011755, RCV000083607 |
| | TRANSCARBAMYLASE | | | |
| | DEFICIENCY | | | |
| .0024 | ORNITHINE | OTC, TYR313ASP | rs66469337 | RCV000011756, RCV000083325 |
| | TRANSCARBAMYLASE | | | |
| | DEFICIENCY | | | |
| .0025 | ORNITHINE | OTC, ARG129HIS | rs66656800 | RCV000011757, RCV000083414 |
| | TRANSCARBAMYLASE | | | |
| | DEFICIENCY | | | |
| .0026 | ORNITHINE | OTC, LEU148PHE | rs66741318 | RCV000083441, RCV000011758 |
| | TRANSCARBAMYLASE | | | |
| | DEFICIENCY | | | |
| .0027 | ORNITHINE | OTC, MET206ARG | rs72558412 | RCV000011759, RCV000083513 |
| | TRANSCARBAMYLASE | | | |
| | DEFICIENCY | | | |
| .0028 | ORNITHINE | OTC, ARG40CYS | rs72554307 | RCV000083332, RCV000011760 |
| | TRANSCARBAMYLASE | | | |
| | DEFICIENCY | | | |
| .0029 | ORNITHINE | OTC, ARG40HIS | rs72554308 | RCV000083333, RCV000011761 |
| | TRANSCARBAMYLASE | | | |
| | DEFICIENCY | | | |

A more expansive list of the mutations, insertions and/or deletions associated with OTCD is provided, e.g., at http://www.uniprot.org/uniprot/P00480, which is incorporated by reference herein. Suitable target sites for correcting other errors in this and/or other disorders may be selected. For example, www.uniprot.org/uniprot provides a list of mutations associated with other diseases, e.g., cystic fibrosis [www.uniprot.org/uniprot/P13569; also OMIM: 219700], MPSIH [http://www.uniprot.org/uniprot/P35475; OMIM:607014]; hemophilia B [Factor IX, http://www.uniprot.org/uniprot/P00451]; hemophilia A [Factor VIII, http://www.uniprot.org/uniprot/P00451]. Still other diseases and associated mutations, insertions and/or deletions can be obtained from reference to this database. Other suitable sources may include, e.g., http://www.genome.gov/10001200; http://www.kumc.edu/gec/support/; http://www.ncbi.nlm.nih.gov/books/NBK22183/.

The target sites are selected such that they do not disrupt expression of functional portions of the gene. Optionally, more than one correction may be made to a target gene using the system described herein. Suitably, the vectors delivering donor template which are gene fragments are designed such that the donor template is inserted upstream of the gene mutation or phenotype to be corrected.

As an alternative, a full-length functioning gene may be inserted into the genome to replace the defective gene. Thus, in one embodiment, the inserted sequence may be a full-length gene, or a gene encoding a functional protein or enzyme. Where a full-length gene is being delivered, there is more flexibility within the target gene for targeting. As another alternative, a single exon may be inserted upstream of the defective exon. In another alternative, gene deletion or insertion can be corrected.

In still another embodiment, the compositions described herein are used to reduce expression of a gene having undesirably high expression levels. Such a gene may be a PCSK9 which binds to the receptor for low-density lipoprotein (LDL) cholesterol; reducing PCSK9 expression can be used to increase circulating LDL cholesterol levels. Still other genes for targeting cancer-associated genes. (e.g., BRCA1, BRCA2). See, also, http://www.eupedia.com/genetics/cancer_related_snp.shtml.

The dual AAV vector system described in detail herein expresses sgRNA and donor template RNA for precise in vivo gene correction of a metabolic liver disorder characterized by a genetic abnormality in the liver (hepatocytes). This system is particularly well suited for neonatal vector delivery. In one embodiment, neonatal treatment is defined as delivering treatment within 8 hours, the first 12 hours, the first 24 hours, or the first 48 hours following delivery, or up about 28 days. In another embodiment, particularly for a primate, neonatal delivery is within the period of about 12 hours to about 1 week, 2 weeks, 3 weeks, or about 1 month, or after about 24 hours to about 48 hours. Optionally, the system may be used for pre-natal delivery, delivery in infants, older children and/or adults. The same system can also be adapted to correct a variety of disorders when appropriate donor sequences and sgRNAs are incorporated into the system. Corresponding changes may also be made to the selection of vector elements, including the selection of the AAV capsid and the selection of a different type of vector system.

In one study performed to date, a dual AAV8 vector system has been used to express sgRNA and donor template DNA for precise in vivo gene correction of an OTC mutation about 25% of hepatocytes following neonatal vector delivery. This is believed to be the first time that efficient gene delivery and CRISPR mediated gene correction in hepatocytes has been demonstrated using an AAV vector system.

Although AAV8 is described herein in the working examples, a variety of different AAV capsids have been described and may be used, although AAV which preferentially target the liver and/or deliver genes with high efficiency are particularly desired. The sequences of the AAV8 have been described in, e.g., U.S. Pat. No. 7,790,449; U.S. Pat. No. 7,282,199, and are available from a variety of databases. While the examples utilize AAV vectors having the same capsid, the capsid of the gene editing vector and the AAV targeting vector are the same AAV capsid. Another suitable AAV may be, e.g., rh10 [WO 2003/042397]. Still other AAV sources may include, e.g., AAV9 [U.S. Pat. No. 7,906,111; US 2011-0236353-A1], and/or hu37 [see, e.g., U.S. Pat. No. 7,906,111; US 2011-0236353-A1], AAV1, AAV2, AAV3, AAV4, AAV5, AAV6, AAV6.2, AAV7, AAV8, [U.S. Pat. No. 7,790,449; U.S. Pat. No. 7,282,199] and others. See, e.g., WO 2003/042397; WO 2005/033321, WO 2006/110689; U.S. Pat. No. 7,790,449; U.S. Pat. No. 7,282,199; U.S. Pat. No. 7,588,772B2 for sequences of these and other suitable AAV, as well as for methods for generating AAV vectors. Still other AAV may be selected, optionally taking into consideration tissue preferences of the selected AAV capsid.

A recombinant AAV vector (AAV viral particle) may comprise, packaged within an AAV capsid, a nucleic acid molecule containing a 5′ AAV ITR, the expression cassettes described herein and a 3′ AAV ITR. As described herein, an expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

A single-stranded AAV viral vector may be used. Methods for generating and isolating AAV viral vectors suitable for delivery to a subject are known in the art. See, e.g., U.S. Pat. No. 7,790,449; U.S. Pat. No. 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In another embodiment, other viral vectors may be used, including integrating viruses, e.g., herpesvirus or lentivirus, although other viruses may be selected. Suitably, where one of these other vectors is generated, it is produced as a replication-defective viral vector. A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production.

A variety of different diseases and conditions associated with one or more genetic deletions, insertions or mutations, may be treated using the method described herein. Examples of such conditions may include, e.g., alpha-1-antitrypsin deficiency, liver conditions (e.g., biliary atresia, Alagille syndrome, alpha-1 antitrypsin, tyrosinemia, neonatal hepatitis, Wilson disease), metabolic conditions such as biotinidase deficiency, carbohydrate deficient glycoprotein syndrome (CDGS), Crigler-Najjar syndrome, diabetes insipidus, Fabry, galactosemia, glucose-6-phosphate dehydrogenase (G6PD), fatty acid oxidation disorders, glutaric aciduria, hypophosphatemia, Krabbe, lactic acidosis, lysosomal storage diseases, mannosidosis, maple syrup urine, mitochondrial, neuro-metabolic, organic acidemias, PKU, purine, pyruvate dehydrogenase deficiency, urea cycle conditions, vitamin D deficient, and hyperoxaluria. Urea cycle disorders include, e.g., N-acetylglutamate synthase deficiency, carbamoyl phosphate synthetase I deficiency, ornithine transcarbamylase deficiency, “AS deficiency” or citrullinemia, “AL deficiency” or argininosuccinic aciduria, and “arginase deficiency” or argininemia.

Other diseases may also be selected for treatment according to the method described herein. Such diseases include, e.g., cystic fibrosis (CF), hemophilia A (associated with defective factor VIII), hemophilia B (associated with defective factor IX), mucopolysaccharidosis (MPS) (e.g., Hunter syndrome, Hurler syndrome, Maroteaux-Lamy syndrome, Sanfilippo syndrome, Scheie syndrome, Morquio syndrome, other, MPSI, MPSII, MPSIII, MSIV, MPS 7); ataxia (e.g., Friedreich ataxia, spinocerebellar ataxias, ataxia telangiectasia, essential tremor, spastic paraplegia); Charcot-Marie-Tooth (e.g., peroneal muscular atrophy, hereditary motor sensory neuropathy), glycogen storage diseases (e.g., type I, glucose-6-phosphatase deficiency, Von Gierke), II (alpha glucosidase deficiency, Pompe), III (debrancher enzyme deficiency, Cori), IV (brancher enzyme deficiency, Anderson), V (muscle glycogen phosphorylase deficiency, McArdle), VII (muscle phosphofructokinase deficiency, Tauri), VI (liver phosphorylase deficiency, Hers), IX (liver glycogen phosphorylase kinase deficiency). This list is not exhaustive and other genetic conditions are identified, e.g., www.kumc.edu/gec/support; http://www.genome.gov/10001200; and http://www.ncbi.nlm.nih.gov/books/NBK22183/, which are incorporated herein by reference.

Other disorders include a central nervous system (CNS)-related disorder. As used herein, a “CNS-related disorder” is a disease or condition of the central nervous system. Such a disorder may affect the spinal cord, brain, or tissues surrounding the brain and spinal cord. Non-limiting examples of CNS-related disorders, include Parkinson's Disease, Lysosomal Storage Disease, Ischemia, Neuropathic Pain, Amyotrophic lateral sclerosis (ALS) (e.g., linked to a mutation in the gene coding for superoxide dismutase, SOD1), Multiple Sclerosis (MS), and Canavan disease (CD), or a primary or metastatic cancer.

In another embodiment, cells of the retina are targeted, including retinal pigment epithelium (RPE) and photoreceptors, e.g., for treatment of retinitis pigmentosa and/or Leber congenital amaurosis (LCA). Optionally, this treatment may utilize or follow subretinal injection and/or be used in conjunction with the standard of care for the condition.

In one aspect, the method is useful in treating a disorder, comprising: co-administering to a subject having the disorder. The dual AAV system employs (a) a gene editing AAV vector comprising a Cas9 (or Cpf1) gene under control of regulatory sequences which direct its expression in a target cell (e.g., a hepatocyte or lung cell) comprising a targeted gene which has one or more mutations resulting in a disorder; and (b) an AAV targeting vector comprising sgRNA and donor template, wherein the sgRNA comprises at least 20 nucleotides which specifically bind to a selected site in the targeted genes and is 5′ (or 3′) to a PAM which is specifically recognized by the Cas9 (or Cpf1), and wherein the donor template comprises nucleic acid sequences which replaces at least one of the mutations in the targeted gene and PAM sequences are mutated; wherein the ratio of gene editing AAV vector of (a) to (b) is such that (b) is in excess of (a). Also provided is use of the vector system as described herein to treat a disorder in humans, or in the preparation of a medicament for the treatment of a disorder.

In one embodiment, the method is used in neonates or infants. Alternatively, the method is used in older subjects. In one aspect, the invention provides a method of treating a liver metabolic disorder in neonates, comprising: co-administering to a subject having a liver metabolic disorder. The dual AAV system employs (a) a gene editing AAV vector comprising a Cas9 (or Cpf1) gene under control of regulatory sequences which direct its expression in a hepatocyte comprising a targeted gene which has one or more mutations resulting in a liver metabolic disorder; and (b) an AAV targeting vector comprising sgRNA and donor template, wherein the sgRNA comprises at least 20 nucleotides which specifically bind to a selected site in the targeted genes and is 5′ or 3′ to a protospacer-adjacent motif (PAM) which is specifically recognized by the enzyme, and wherein the donor template comprises nucleic acid sequences which replaces at least one of the mutations in the targeted gene and the PAM corresponding to the sgRNA is mutated; wherein the ratio of gene editing AAV vector of (a) to (b) is such that (b) is in excess of (a). Also provided is the use of (a) a gene editing AAV vector comprising a Cas9 gene under control of regulatory sequences which direct its expression in a hepatocyte comprising a targeted gene which has one or more mutations resulting in a liver metabolic disorder; and (b) an AAV targeting vector comprising sgRNA and donor template, wherein the sgRNA comprises at least 20 nucleotides which specifically bind to a selected site in the targeted genes and which is 5′ to a protospacer-adjacent motif (PAM) which is specifically recognized by the Cas9, and wherein the donor template comprises nucleic acid sequences which replaces at least one of the mutations in the targeted gene, to treat a liver metabolic disorder in a neonate subject, wherein the ratio of gene editing AAV vector of (a) to (b) is such that (b) is in excess of (a), or the use of these vectors in the preparation of a medicament for the treatment of a liver metabolic disorder.

In one embodiment, the ratio of editing vector to targeting vector is about 1:3 to about 1:100, inclusive of intervening ratios. For example, the ratio of editing vector to targeting vector may be about 1:5 to about 1:50, or about 1:10, or about 1:20. Although not as preferred, the ratio may be 1:1 or there may be more targeting vector.

In general, the ratio of AAV vectors is determined based on particle copies (pt) or genome copies (GC), which terms may be used interchangeably herein, for each vector. Suitably, when determining the ratio of two or more AAV vectors to one another (e.g., editing vector to targeting vector), the same method is used to determine the number of each type of vector(s). However, if different methods are determined to be substantially equivalent, different techniques may be used. Suitable method for determining genome copies (GC) have been described and include, e.g., oqPCR or digital droplet PCR (ddPCR) as described in, e.g., M. Lock et al, Hu Gene Therapy Methods, Hum Gene Ther Methods. 2014 April; 25(2):115-25. doi: 10.1089/hgtb.2013.131. Epub 2014 Feb. 14, which is incorporated herein by reference.

While unlike traditional gene augmentation therapy, gene editing-mediated correction as described herein in neonates may not require readministration. However, optionally, a second or subsequent additional treatments involving co-administration of the dual vector Crispr/enzyme system provided herein may be pursued. For example, where patients are treated as neonates and/or the targeted cells are proliferating cells (e.g., liver cells, epithelial cells, or cancer cells), subsequent treatments may be desired. Such subsequent treatments may follow the first treatment by a month, several months, a year, or several years. Optionally, the subsequent treatment may utilize vectors having different capsids than were utilized for the initial treatment. For example, if initial treatment was by AAV8, a second treatment may utilize rh10. In another example, if initial treatment utilized rh10, subsequent treatment may utilize AAV8. Still other combinations of AAV capsids may be selected by one skilled in the art.

The compositions described herein are designed for delivery to subjects in need thereof by any suitable route or a combination of different routes. For treatment of liver disease, direct or intrahepatic delivery to the liver is desired and may optionally be performed via intravascular delivery, e.g., via the portal vein, hepatic vein, bile duct, or by transplant. Alternatively, other routes of administration may be selected (e.g., oral, inhalation, intranasal, intratracheal, intraarterial, intraocular, intravenous, intramuscular, and other parental routes). For example, intravenous delivery may be selected for delivery to proliferating, progenitor and/or stem cells. Alternatively, another route of delivery may be selected. The delivery constructs described herein may be delivered in a single composition or multiple compositions. Optionally, two or more different AAV may be delivered [see, e.g., WO 2011/126808 and WO 2013/049493]. In another embodiment, such the dual vector system may contain only a single AAV and a second, different, Cas9-delivery system. For example, Cas9 (or Cpf1) delivery may be mediated by non-viral constructs, e.g., “naked DNA”, “naked plasmid DNA”, RNA, and mRNA; coupled with various delivery compositions and nanoparticles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, both of which are incorporated herein by reference. Such non-viral delivery constructs may be administered by the routes described previously.

The viral vectors, or non-viral DNA or RNA transfer moieties can be formulated with a physiologically acceptable carrier for use in gene transfer and gene therapy applications. In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). The replication-defective virus compositions can be formulated in dosage units to contain an amount of replication-defective virus that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of replication-defective virus in the formulation is 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, or 1.0×10¹³ GC, 5.0×10¹³ GC, 1.0×10¹⁴ GC, 5.0×1014 GC, or 1.0×10¹⁵ GC.

Production of lentivirus is measured as described herein and expressed as IU per volume (e.g., mL). IU is infectious unit, or alternatively transduction units (TU); IU and TU can be used interchangeably as a quantitative measure of the titer of a viral vector particle preparation. The lentiviral vector is typically integrating. The amount of viral particles is at least about 3×10⁶ IU, and can be at least about 1×10⁷ IU, at least about 3×10⁷ IU, at least about 1×10⁸ IU, at least about 3×10⁸ IU, at least about 1×10⁹ IU, or at least about 3×10⁹ IU.

In addition, the dual vector system described herein may involve co-administration of an AAV vector as described herein in combination with a non-AAV vector carrying the enzyme (Cas9 or Cpf1). For example, the enzyme may be delivered via a different vector, or via mRNA or DNA alone, or in combination with AAV vector-mediated delivery of the Cas9. For example, a Cas9 (or Cpf1) sequence may be via a carrier system for expression or delivery in RNA form (e.g., mRNA) using one of a number of carrier systems which are known in the art. Such carrier systems include those provided by commercial entities, such as PhaseRx′ so-called “SMARTT” technology. These systems utilize block copolymers for delivery to a target host cell. See, e.g., US 2011/0286957 entitled, “Multiblock Polymers”, published Nov. 24, 2011; US 2011/0281354, published Nov. 17, 2011; EP2620161, published Jul. 31, 2013; and WO 2015/017519, published Feb. 5, 2015. See, also, S. Uchida et al, (February 2013) PLoS ONE 8(2): e56220. Still other methods involve generating and injecting synthetic dsRNAs [see Soutschek et al. Nature (2004) 432(7014): 173-8; see also Morrissey et al. Hepatol. (2005) 41(6): 1349-56], local administration to the liver has also been demonstrated by injecting double stranded RNA directly into the circulatory system surrounding the liver using renal vein catheterization. [See Hamar et al. PNAS (2004) 101(41): 14883-8.]. Still other systems involve delivery of dsRNA and particularly siRNA using cationic complexes or liposomal formulations [see, e.g., Landen et al. Cancer Biol. Ther. (2006) 5(12); see also Khoury et al. Arthritis Rheumatol. (2006) 54(6): 1867-77. Other RNA delivery technologies are also available, e.g., from Veritas Bio [see, e.g., US 2013/0323001, Dec. 23, 2010, “In vivo delivery of double stranded RNA to a target cell” (cytosolic content including RNAs, e.g., mRNA, expressed siRNA/shRNA/miRNA, as well as injected/introduced siRNA/shRNA/miRNA, or possibly even transfected DNA present in the cytosol packaged within exovesicles and be transported to distal sites such as the liver)]. Still other systems for in vivo delivery of RNA sequences have been described. See, e.g., US 2012/0195917 (Aug. 2, 2012) (5′-cap analogs of RNA to improve stability and increase RNA expression), WO 2013/143555A1, Oct. 3, 2013, and/or are commercially available (BioNTech, Germany; Valera (Cambridge, Mass.); Zata Pharmaceuticals).

DNA and RNA are generally measured in the nanogram (ng) to microgram (μg) amounts of the nucleic acids. In general, for a treatment in a human preferably dosages of the RNA is the range of 1 ng to 700 μg, 1 ng to 500 μg, 1 ng to 300 μg, 1 ng to 200 μg, or 1 ng to 100 μg are formulated and administered. Similar dosage amounts of a DNA molecule (e.g., containing a Cas9 or other expression cassette) and not delivered to a subject via a viral vector may be utilized for non-viral DNA delivery constructs.

The above-described recombinant vectors or other constructs may be delivered to host cells according to published methods. The vectors or other moieties are preferably suspended in a physiologically compatible carrier, may be administered to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The system described herein may be therapeutically useful if a sufficient amount of functional enzyme or protein is generated to improve the patient's condition. In certain embodiments, gene expression levels as low as 5% of healthy patients will provide sufficient therapeutic effect for the patient to be treatable to non-gene therapy approaches. In other embodiments, gene expression levels are at least about 10%, at least about 15% to up to 100% of the normal range (levels) observed in humans (other veterinary subject). For example, by “functional enzyme”, is meant a gene which encodes the wild-type enzyme (e.g., OTCase) which provides at least about 50%, at least about 75%, at least about 80%, at least about 90%, or about the same, or greater than 100% of the biological activity level of the wild-type enzyme, or a natural variant or polymorph thereof which is not associated with disease. More particularly, as heterozygous patients may have as low an enzyme functional level as about 50% or lower, effective treatment may not require replacement of enzyme activity to levels within the range of “normal” or non-deficient patients. Similarly, patients having no detectable amounts of enzyme may be rescued by delivering enzyme function to less than 100% activity levels, and may optionally be subject to further treatment subsequently. In certain embodiments, were gene function is being delivered by the donor template, patients may express higher levels than found in “normal”, healthy subjects. In still other embodiments, where reduction in gene expression is desired, as much as a 20% reduction to a 50% reduction, or up to about 100% reduction, may provide desired benefits. As described herein, the therapy described herein may be used in conjunction with other treatments, i.e., the standard of care for the subject's (patient's) diagnosis.

A variety of assays exist for measuring OTC expression and activity levels in vitro. See, e.g., X Ye, et al, 1996 Prolonged metabolic correction in adult ornithine transcarbamylase-deficient mice with adenoviral vectors. J Biol Chem 271:3639-3646) or in vivo. For example, OTC enzyme activity can be measured using a liquid chromatography mass spectrometry stable isotope dilution method to detect the formation of citrulline normalized to [1,2,3,4,5-13C5] citrulline (98% 13C). The method is adapted from a previously developed assay for detection of N-acetylglutamate synthase activity [Morizono H, et al, Mammalian N-acetylglutamate synthase. Mol Genet Metab. 2004; 81(Suppl 1):S4-11]. Slivers of fresh frozen liver are weighed and briefly homogenized in buffer containing 10 mM HEPES, 0.5% Triton X-100, 2.0 mM EDTA and 0.5 mM DTT. Volume of homogenization buffer is adjusted to obtain 50 mg/ml tissue. Enzyme activity is measured using 250 μg liver tissue in 50 mM Tris-acetate, 4 mM ornithine, 5 mM carbamyl phosphate, pH 8.3. Enzyme activity is initiated with the addition of freshly prepared 50 mM carbamyl phosphate dissolved in 50 mM Tris-acetate pH 8.3, allowed to proceed for 5 minutes at 25° C. and quenched by addition of an equal volume of 5 mM13C5-citrulline in 30% TCA. Debris is separated by 5 minutes of microcentrifugation, and the supernatants are transferred to vials for mass spectroscopy. Ten μL of sample are injected into an Agilent 1100 series LC-MS under isocratic conditions with a mobile phase of 93% solvent A (1 ml trifluoroacetic acid in 1 L water):7% solvent B (1 ml trifluoroacetic acid in 1 L of 1:9 water/acetonitrile). Peaks corresponding to citrulline [176.1 mass:charge ratio (m/z)] and 13C5-citrulline (181.1 m/z) are quantitated, and their ratios are compared to ratios obtained for a standard curve of citrulline run with each assay. Samples are normalized to either total liver tissue or to protein concentration determined using a Bio-Rad protein assay kit (Bio-Rad, Hercules, Calif.). Other assays, which do not require liver biopsy, may also be used. One such assay is a plasma amino acid assays in which the ratio of glutamine and citrulline is assessed and if glutamine is high (>800 microliters/liter) and citrulline low (e.g., single digits), a urea cycle defect is suspected. Plasma ammonia levels can be measured and a concentration of about 100 micromoles per liter is indicative of OTCD. Blood gases can be assessed if a patient is hyperventilating; respiratory alkalosis is frequent in OTCD. Orotic acid in urine, e.g., greater than about 20 micromoles per millimole creatine is indicative of OTCD, as is elevated urinary orotate after allopurinol challenge test. Diagnostic criteria for OTCD have been set forth in Tuchman et al, 2008, Urea Cycle Disorders Consortium (UCDC) of the Rare Disease Clinical Research Network (RDCRN). Tuchman M, et al., Consortium of the Rare Diseases Clinical Research Network. Cross-sectional multicenter study of patients with urea cycle disorders in the United States. Mol Genet Metab. 2008; 94:397-402, which is incorporated by reference herein. See, also, http://www.ncbi.nlm.nih.gov/books/NBK154378/, which provides a discussion of the present standard of care for OTCD.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% (±10%) from the reference given, unless otherwise specified.

A “subject” is a mammal, e.g., a human, mouse, rat, guinea pig, dog, cat, horse, cow, pig, or non-human primate, such as a monkey, chimpanzee, baboon or gorilla. A patient refers to a human. A veterinary subject refers to a non-human mammal.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not a limitation on the invention described herein.

### EXAMPLES

### Example 1—Correction of OTC Deficiency Using Dual AAV CRISPR-Cas9

An X-linked deficiency of the OTC enzyme in humans causes recurrent and life threatening episodes of hyperammonemia [Batshaw, M., et al, Mol Genet Metab, 113: 127-139 (2014); Lichter-Koneti, U, et al., Ornithine Transcarbamylase Deficiency (1993-2013) In: Pagon R A, Adam M P, Ardinger H H, et al., editors. GeneReviews® (http://www.ncbi.nlm.nih.gov/books/NBK154378/)]. In males hemizygous for OTC deficiency, the first metabolic crisis can usually occur in the newborn period and is associated with up to 50% mortality; survivors typically undergo liver transplantation in the first year of life [Ah Mew et al, J Pediatr, 162: 324-329, e321 (2013)]. An animal model of OTC deficiency, the male sparse fur ash (spf^{ash}) mouse, has a point mutation at the 3 donor splice site at the end of exon 4 of the OTC gene which leads to abnormal splicing and a 20-fold reduction in OTC mRNA and protein [Hodges, P. E. & Rosenberg, L. E. The spf^{ash} mouse: a missense mutation in the ornithine transcarbamylase gene also causes aberrant mRNA splicing. Proc Natl Acad Sci USA 86, 4142-4146 (1989)]. Affected animals can survive on a chow diet, but develop hyperammonemia that can be lethal when provided on a high protein diet.

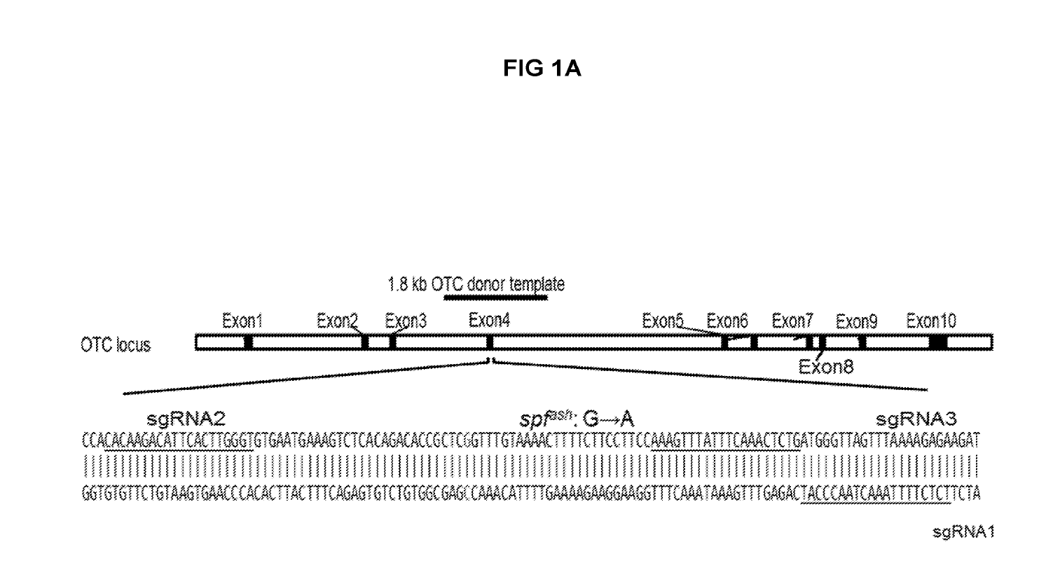

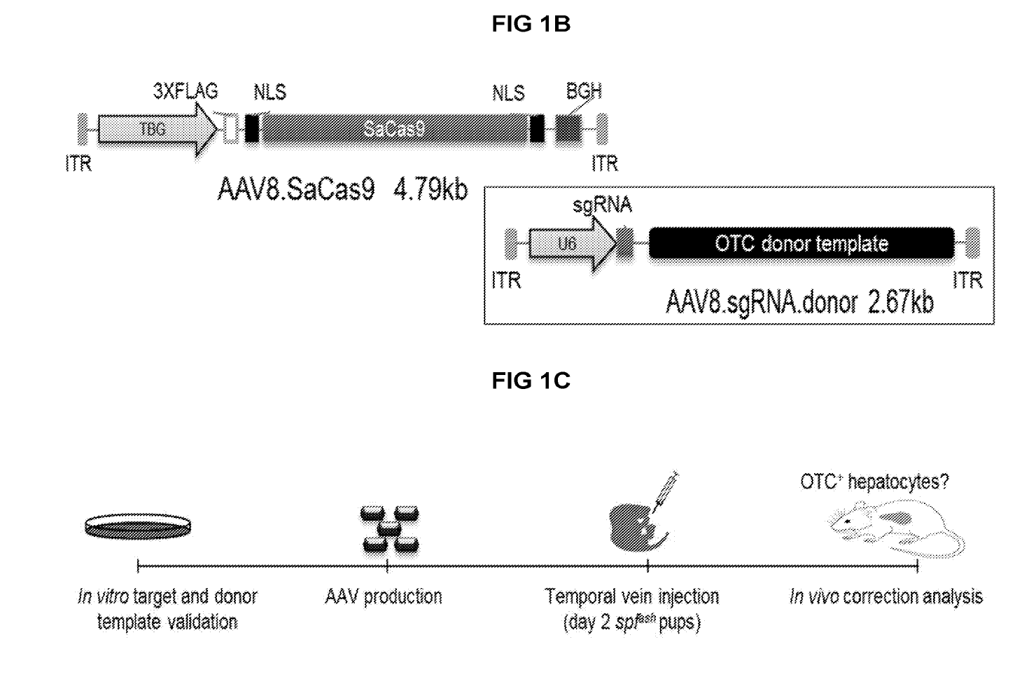

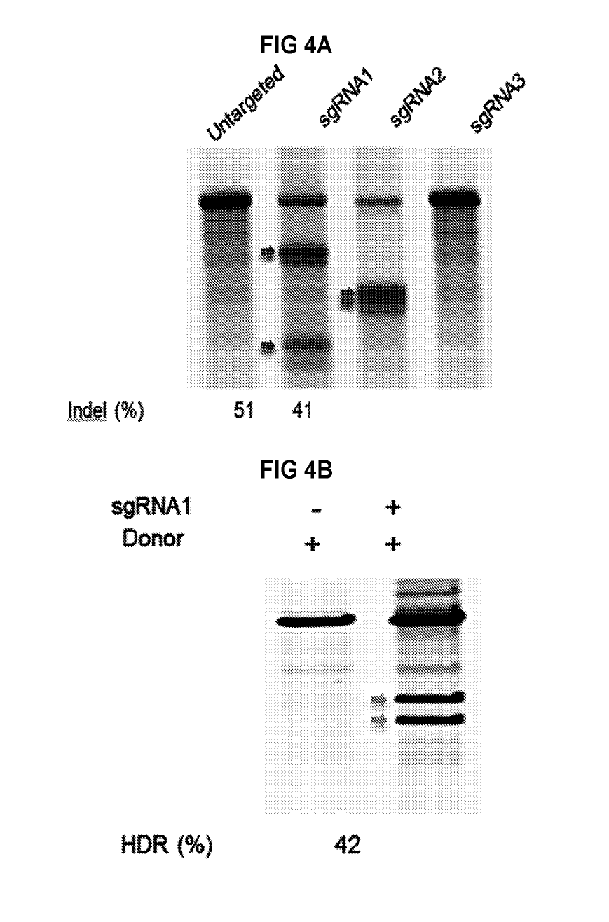

The studies in this Example utilized an AAV vector with high liver tropism (i.e., AAV8) to correct the point mutation in newborn spf^{ash} mice using Cas9 enzymes from Streptococcus pyogenes (SpCas9) [Jinek, M. et al, Science, 337: 816-821 (2012); Cong L., et al, Science, 339: 819-823 (2013); Mali P, et al, Science, 339: 823-826 (2013)] and Staphylococcus aureus (SaCas9) [Ran, F A, et al., Nature, 520: 186-191 (2015)]. Protospacer-adjacent motif (PAM) sequences (NNGRRT) in proximity to the spf^{ash} mutation of the OTC gene were identified, as were potential 20 nt guide sequences. Three sequences, sgRNA1-3 (FIG. 1A), were further evaluated following transfection of neomycin-containing plasmids into a mouse MC57G cell line. Low transfection efficiency in this cell line required enrichment of transfected cells by a brief exposure to puromycin. Evidence for double-strand breaks (DSBs) at the desired site was demonstrated using the SURVEYOR assay with two of the three guide RNAs (FIG. 4A). Due to the absence of indel formation with sgRNA3, this guide sequence was not pursued further. The remaining candidates, sgRNA1 and sgRNA2, were similar in both DSB induction efficiency (FIG. 4A) and proximity to the spf^{ash} mutation (FIG. 1A). Due to its location within exon 4, sgRNA2 was avoided; DSB induction within an exon can lead to non-homologous end joining (NHEJ) without homology directed repair (HDR), which could ablate the residual OTC activity characteristic of the spf^{ash} mutation, thereby reducing residual ureagenesis. As a result, intronic sgRNA1 was selected for further experimentation. The sgRNA1 guide was then transfected with SaCas9 plus a donor DNA with approximately 0.9 kb of sequence flanking each side of the mutation in which the corresponding PAM sequence was mutated and an AgeI site was included to facilitate detection of HDR. High efficiency HDR was achieved with this combination of SaCas9, donor, and sgRNA1 (FIG. 4B).

A two-vector approach was necessary to incorporate all components into AAV (FIG. 1B). Vector 1 expresses the SaCas9 gene from a liver specific promoter called TBG (subsequently referred to as AAV8.SaCas9), while vector 2 contains sgRNA1 sequence expressed from a U6 promoter and the 1.8 kb donor OTC DNA sequence (referred to as AAV8.sgRNA1.donor). In all experiments, spf^{ash} pups were injected IV on postnatal day 2 with mixtures of vector 1 and vector 2 and subsequently evaluated for indel formation and functional correction of the spf^{ash} mutation (FIG. 1C).

To generate a dual AAV vector system for in vivo OTC gene correction by SaCas9, we constructed two AAV cis-plasmids: 1) the hSaCas9 was subcloned from pX330.hSaCas9 into an AAV backbone plasmid containing the full length TBG promoter (two copies of enhancer elements of the a microglobulin/bikunin gene followed by a liver-specific TBG promoter) and the bovine growth hormone polyadenylation sequence; 2) the 1.8-kb OTC donor template was cloned into the pAAV backbone and the U6-OTC sgRNA sequence was inserted into the AflII site (FIG. 1C), yielding AAV8.sgRNA1 donor. The PAM sequence on the donor template AAV.sgRNA1donor was mutated (Table 1) to prevent re-cleavage by Cas9 after HDR, and an AgeI site was added to facilitate detection of HDR. The untargeted AAV8.control.donor differs from the targeted AAV8.sgRNA1 donor by eliminating the protospacer from the U6-OTC sgRNA cassette. Puromycin-resistant gene was cloned into pX330.hSaCas9-derived plasmids for selection of transfected cells following in vitro transient transfection. All plasmid constructs were verified by sequencing.

A. Vector Construction

To edit the murine OTC locus with hSpCas9 or hSaCas9, 20-nt target sequences, e.g., the hSpCas9 or hSaCas9 target illustrated in in the table below and FIG. 1 were selected to precede a 5′-NGG protospacer-adjacent motif (PAM) or 5′-NNGRRT PAM sequence. FIG. 1 shows SaCas9 target sites.

TABLE 2

| | PAM Signature | Source of |
|---|---|---|
| Target sequence | Sequence | Cas9 |
| AGTTTGAAATAAACTTTGGA | AGG | SpCas9 |
| (SEQ ID NO: 5) | | |
| GAAAAGTTTTACAAACTGAG | CGG | SpCas9 |
| (SEQ ID NO: 6) | | |
| TCAGAGTTTGAAATAAACTT | TGG | SpCas9 |
| (SEQ ID NO: 7) | | |
| TCTCTTTTAAACTAACCCAT | CAGAGT | SaCas9 |
| (SEQ ID NO: 8) | | |
| CACAAGACATTCACTTGGGT | GTGAAT | SaCas9 |
| (SEQ ID NO: 9) | | |
| AAAGTTTATTTCAAACTCTG | ATGGGT | SaCas9 |
| (SEQ ID NO: 10) | | |

Three target sequences were selected and individually cloned into the pX330 plasmid (Addgene), which co-expresses the sgRNA and humanized SpCas9 (hSpCas9). The OTC donor template plasmid was constructed by amplifying a 1.8-kb fragment flanking the G/A mutation site in spf^{ash} mouse using genomic DNA isolated from a C57BL/6 mouse as the template. The AgeI restriction site was subsequently introduced into donor template with the In-Fusion® HD Cloning System (Clontech). To generate a dual AAV vector system for in vivo OTC gene editing (hSpCas9), two AAV cis-plasmids were constructed: 1) the hSpCas9 was subcloned from pX330 into a AAV backbone plasmid containing two copies of shortened enhancer elements of α microglobulin/bikunin gene [http://www.ncbi.nlm.nih.gov/gene/259, accessed Apr. 24, 2015] followed by a shortened liver-specific TBG promoter (TBG-S1) and a minimal polyadenylation signal (PA75) (signal pattern: AATAAA) [GB Accession number CN480585, https://fasterdb.lyon.unicancer.fr]; 2) the 1.8-kb OTC donor template was cloned into pAAV backbone and U6-OTC sgRNA sequence was inserted into the NW site.

The smaller size of Cas9 from Staphylococcus aureus (SaCas9) made it desirable for packaging into the AAV vector. FLAG-tagged SaCas9 was codon-optimized according to codon usage in human (hSaCas9) and pX330.hSaCas9 was constructed by replacing the hSpCas9 and sgRNA scaffold in pX330 with hSaCas9 and SaCas9 sgRNA scaffold. Three 20-nt target sequences preceding a 5′NNGRRT PAM sequence were selected for OTC gene editing.

Illustrative plasmid sequences are provided in the attached Sequence Listing and include, pAAV.ABPS2.TBG-S1.hSpCas9; pAAV. TBG.hSaCas9.bGH; pAAV.U6.control.mOTC.T1.9(hSaCas9); pAAV.U6.control.mOTC.T1.8(hSpCas9); pAAV.U6.0TC\sgRNA1.mOTC.T1.8.MfeI\(hSaCas9)\; pAAV.U6.OTC\sgRNA1.mOTC.T1.8.MfeI (hSpCas9); pAAV.U6.0TC\sgRNA1.mOTC.T1.8.TBG.hOTCco.BGH(hSaCas9); pAAV.U6.OTC\sgRNA2.mOTC.T1.8.M2\(hSaCas9)\; and pAAV.U6.mOTC\sgRNA4.mOTC.T1.8.MfeI (hSpCas9)\.

B. In Vitro Validation of OTC sgRNAs and Donor Template

MC57G cells were transiently transfected with OTC targeted Cas9 plasmid (250 ng) with increasing amounts of AgeI tagged OTC donor plasmid (250, 500, 1000 ng) followed by 4-day puromycin enrichment and SURVEYOR nuclease assay. SpCas9 sgRNA3 and SaCas9 sgRNA1 showed the highest efficiency in inducing indels in the targeted loci and therefore were chosen for subsequent studies.

Increasing the amount of donor template did not result in increasing levels of HDR in vitro. Co-transfection of untargeted Cas9 plasmid (250 ng) and the AgeI tagged OTC donor template (1000 ng) did not result in detectable HDR. Arrows denote AgeI-sensitive cleavage products resulting from HDR. Lanes with no quantification had no detectable Indels or HDR.

C. Cell Culture and Transfection

MC57G cells (ATCC) were maintained in DMEM medium supplemented with 10% FBS and cultured at 37° C. with 5% CO₂. Cell lines were used directly upon receipt from ATCC and were not authenticated or tested for mycoplasma contamination. For in vitro target and/or donor template testing, plasmids were transfected into MC57G cells using Lipofectamine® LTX with Plus™ reagent (Life Technology) per manufacturer's recommendations. Transfected cells were under puromycin (4 μg mL⁻¹) selection for 4 days to enrich transfected cells.

D. Genomic DNA Extraction and SURVEYOR® Assay

Genomic DNA from transfected MC57G cells was extracted using the QuickExtract DNA extraction solution (Epicentre Biotechnologies). The efficiency of individual sgRNA was tested by the SURVEYOR nuclease assay (Transgenomics) using PCR primers listed in Table 3, below, which provides primers and sequences for construction and analysis of the donor template and sgRNA plasmids.

TABLE 3

| | | SEQ | |
|---|---|---|---|
| | | ID | |
| ID | Primer Sequence (5'->3') | NO: | Note |
| WT Sequence | 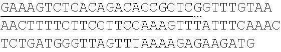 | 11 | 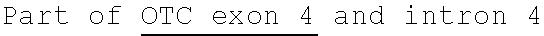 |
| spf^{ash} sequence | 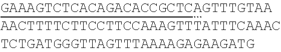 | 12 | 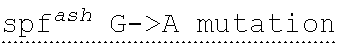 |
| OTC donor | AAAGTCTCACAGACACCGCTCGGTTTGTAAA | 13 | PAM mutation |
| sequence | ACTTTTCTTCCTTGCAAAGTTTATTTCAAACT | | |
| (SpCas9, | CTGATGGGTTAGTTTAAAAGAGAAGATG | | |
| 892-983 bp) | | | |
| OTC donor sequence (SaCas9, 892-989 bp) | 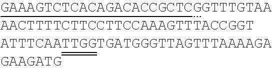 | 14 | 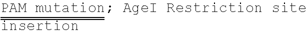 |
| OTC sgRNA1_Fwd | 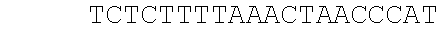 | 15 | OTC target sequence 1 |
| OTC sgRNA1_Rev | 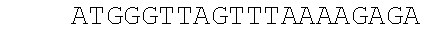 | 16 | OTC target sequence 1 |
| OTC sgRNA2_Fwd | 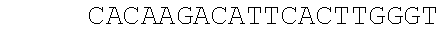 | 17 | OTC target sequence 2 |
| OTC sgRNA2_Rev | 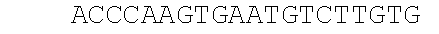 | 18 | OTC target sequence 2 |
| OTC sgRNA3_Fwd | 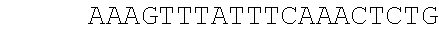 | 19 | OTC target sequence 3 |
| OTC sgRNA3_Rev | 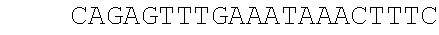 | 20 | OTC target sequence 3 |
| HDR-Fwd | TGGAGCAATTCTGCACATGGA | 21 | OTC PCR for RFLP analysis |
| HDR-Rev | CTTACTGAACATGGCAGTTTCCC | 22 | OTC PCR for RFLP analysis |
| OTC_PointM_F | GGCTATGCTTGGGAATGTCCT | 23 | OTC PCR for Surveyor assay |
| OTC_PointM_R | GCTACAGAATGAAAGAGAGGCG | 24 | OTC PCR for Surveyor assay |
| mOTC hSpCas9 sgRNA1_Fwd | 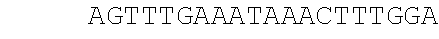 | 25 | mOTC target sequence 1 for hSpCas9 |
| mOTC hSpCas9 sgRNA1_Rev | 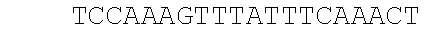 | 26 | |
| mOTC hSpCas9 sgRNA2_Fwd | 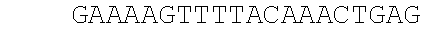 | 27 | mOTC target sequence 2 for hSpCas9 |
| mOTC hSpCas9 sgRNA2_Rev | 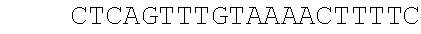 | 28 | |
| mOTC hSpCas9 sgRNA3_Fwd | 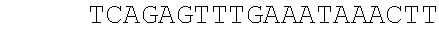 | 29 | mOTC target sequence 3 for hSpCas9 |
| mOTC hSpCas9 sgRNA3_Rev | 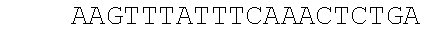 | 30 | |
| mOTC hSaCas9 sgRNA1_Fwd | 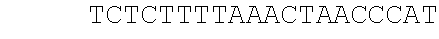 | 31 | mOTC target sequence 1 for hSaCas9 |
| mOTC hSaCas9 sgRNA1_Rev | 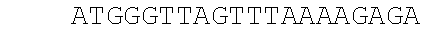 | 32 | |
| mOTC hSaCas9 sgRNA2_Fwd | 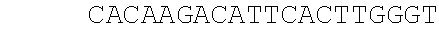 | 33 | mOTC target sequence 2 for hSaCas9 |
| mOTC hSaCas9 sgRNA2_Rev | 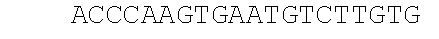 | 34 | |
| mOTC hSaCas9 sgRNA3_Fwd | 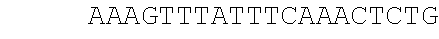 | 35 | mOTC target sequence 3 for hSaCas9 |
| mOTC hSaCas9 sgRNA3_Rev | 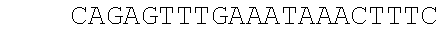 | 36 | |

E. AAV8 Vector Production

All vectors used in this study were packaged with AAV serotype 8 capsid in 293 cells by polyethylenimine (PEI)-based triple transfections, concentrated from the supernatant tangential flow filtration (TFF), and further purified by iodixanol gradient ultracentrifugation as previously described (Lock M et al., 2010). The genome titer (GC/ml⁻¹) of AAV vectors were determined by quantitative PCR (qPCR). All vectors used in this study passed the endotoxin assay using QCL-1000 Chromogenic LAL test kit (Cambrex Bio Science).

F. Animals

spf^{ash} mice were maintained at the Animal Facility at the Translational

Research Laboratories at the University of Pennsylvania as described previously [ML Batshaw, et al, Mol Genet Metab, 113: 127-130 (2014)]. All animal procedures were performed in accordance with protocols approved by the Institutional Animal Care and Use Committee (IACUC) of the University of Pennsylvania. Mating cages were monitored daily for birth. Newborn (postnatal day 2, p2) male pups received a temporal vein injection of the mixture of two vectors at the intended doses for each in a volume of 50 μl, as described [Daly, T M, et al., Methods Mol Biol, 246: 195-199 (2004)]. Untreated WT, spf^{ash} heterozygous (Het), and spf^{ash} hemizygous mice served as controls. Mice were sacrificed at 1, 3, or 8 weeks after vector treatment, and liver samples were harvested for analyses. Mice were genotyped at weaning or at the time of necropsy to confirm genotype.

For testing the efficacy of OTC correction, a high protein diet (40% protein, Animal Specialties & Provisions) was given to 7-week-old mice for 7 days. After this time, plasma was collected for measurement of plasma NH₃ using the Sigma Ammonia Assay Kit. The remaining samples were sent to Antech Diagnostics for measurements of ALT, AST, and total bilirubin.

Note that the entire litter of newborn male pups was injected with either the test or control vectors, and no specific randomization method was used. The following assays were performed in a blinded fashion in which the investigator was unaware of the nature of the vectors or vector dose: vector injection, OTC and Cas9 (FLAG) immunostaining and quantification, histopathology analysis on liver, OTC enzyme activity assay, and gene expression analysis and RT-qPCR.

The adult gene editing experiments were conducted in 8- to 10-week-old male spf^{ash} mice. Animals in low-dose groups received a tail vein injection of AAV8.SaCas9 (1×10¹¹ GC) and AAV8.sgRNA1.donor (1×10¹² GC) or untargeted vectors at the same doses, and they were sacrificed at 3 weeks after injection for analyses. Animals in high-dose groups received a tail vein injection of AAV8.SaCas9 (1×10¹² GC) and AAV8.sgRNA1.donor (5×10¹² GC) or untargeted vectors at the same doses, and they were sacrificed at 2 weeks after injection for analyses.

G. OTC Immunostaining

Immunofluorescence for OTC expression was performed on frozen liver sections. Cryosections (8 μm) were air dried and fixed in 4% paraformaldehyde (all solutions in phosphate-buffered saline) for 10 min Sections were then permeabilized and blocked in 0.2% Triton containing 1% donkey serum for 30 min A rabbit anti-OTC antibody [Augustin, L., et al, Pediatr Res, 48: 842-846 (2000)] diluted 1:1000 in 1% donkey serum was used to incubate the sections for 1 h. After washing, the sections were stained with tetramethylrhodamine (TRITC)-conjugated donkey anti-rabbit antibodies (Vector Labs) in 1% donkey serum for 30 min, washed and mounted with Vectashield (Vector Labs). Some sections were additionally stained with a monoclonal antibody against glutamine synthetase (BD Biosciences, clone 6, Cat#610517) as a marker for pericentral hepatocytes followed by fluorescein isothiocyanate (FITC)-labeled donkey anti-mouse antibodies (Jackson Immunoresearch Laboratories, Cat#715-095-150). Double staining was performed by mixing the two primary and secondary antibodies, respectively, and following the above protocol. Other sections were counterstained with fluorescein-labeled tomato lectin (Lycopersicon esculentum lectin, LEL; Vector Laboratories, Cat#FL-1171) by adding LEL to the secondary antibody solution at a dilution of 1:500.

Cas9 expression was detected on sections from paraffin-embedded livers via immunostaining for FLAG tag using monoclonal antibody M2 (Sigma, Cat#F1804). Paraffin sections were processed according to standard protocols with an antigen retrieval step (boiling for 6 min in 10 mM citrate buffer, pH 6.0). Staining was performed using a mouse-on-mouse (MOM) kit (Vector Laboratories) according to the manufacturer's instructions.

To quantify percentages of OTC-expressing hepatocytes, 10 random images were taken with a 10× objective from each liver section stained for OTC expression. In some cases, where only a small liver section was available, only 5 pictures were taken. Using ImageJ software (Rasband W. S., National Institutes of Health, USA; http://rsb.info.nih.gov/ij/), images were thresholded for OTC-positive area (i.e. the OTC-positive area was selected) and the percentage of the OTC-positive area was determined for each image. In a second measurement the images were thresholded for “empty” area (e.g. veins and sinusoids) to determine the percentage of the area not occupied by liver tissue. This was possible as a result of the presence of weak background fluorescence of the liver tissue. The final percentage of OTC-positive liver tissue (i.e. OTC-positive hepatocytes) was then calculated per adjusted area (total area minus empty area), and the values were averaged for each liver.

To determine the percentage of Cas9-positive hepatocytes, two sections from the each liver were analyzed, one stained for Cas9 (via FLAG tag), the other section stained with hematoxylin to label all nuclei. Three images from every section were taken with an 10× objective and the number of either Cas9-positive or hematoxylin stained hepatocyte nuclei was determined using ImageJ's “Analyze Particles” tool which allows to select and count stained hepatocyte nuclei. Hematoxylin stained nuclei from other cell types could be excluded based on size and circularity parameters. The percentage of Cas9-positive nuclei was then calculated based on the total number of hepatocyte nuclei visible in the hematoxylin-stained sections.

For histochemical staining of OTC activity, sliced liver tissue (2 mm) was fixed, embedded, sectioned (8 μm) and mounted onto slides for histochemical staining of OTC enzyme activity as previously described [Ye, X., et al, J Biol Chem., 271: 3639-3646 (1996)]. Hematoxylin and eosin (H&E) staining was performed on sections from paraffin-embedded liver samples processed and stained according to standard protocols. Sections were analyzed for any abnormalities compared to livers from untreated animals.

H. OTC Enzyme Activity Assay

OTC enzyme activity was assayed in liver lysates as described previously with modifications [Morizono, H, et al., Biochem J., 322 (Pt2): 625-631 (1997)]. Whole-liver fragments were frozen in liquid nitrogen, and stored at −80° C. until OTC measurements were performed. A homogenate of 50 mg liver tissue per mL was prepared in 50 mM Tris acetate buffer pH 7.5, with a Polytron homogenizer (Kinematica AG). A total of 250 μg of liver tissue was used per assay tube, and assays were performed in triplicate. Protein concentration was determined on the remaining liver homogenate using the Bio-Rad Protein assay kit (Bio-Rad) according to the manufacturer's instructions.

I. Western Blot Analysis

Western blot was performed on liver lysate as described previously [Wang, L., et al, Gene Ther, 19: 404-410 (2012)]. OTC protein was detected by a custom rabbit polyclonal antibody (1:10,000 dilution)[Augustin, L., et al, Pediatr Res, 48: 842-846 (2000)]. Mouse anti-FLAG M2 antibody (1:2000 dilution, Sigma) and mouse anti-α-tubulin antibody (1:500 dilution, Santa Cruz) were used to detect Cas9 and α-tubulin. Mouse anti-actin antibody (1:1,000 dilution, Cell Signaling Technology, Cat#8457L) was used to detect actin Blots imaged with ChemiDoc MP system with ImageLab 4.1 software (Bio-Rad).

J. Gene Expression Analysis, RT-PCR and RT-qPCR

RNA was purified using Trizol (Life Technology) and reverse-transcribed using a High-Capacity cDNA Reverse Transcription Kit (Applied Biosystems). Real-time PCR (qPCR) reactions to measure murine OTC, SaCas9 and GAPDH were performed using gene specific primers (Applied Biosystems). Data were normalized to GAPDH.

K. On-Target and Off-Target Mutagenesis Analyses

HDR-mediated targeted modifications were confirmed by restriction-fragment length polymorphism (RFLP) analysis, as described previously [Ran, F., et al, Nat Protocol, 8: 228-2308 (2013)]. The HDR-Fwd and HDR-Rev primers were designed to anneal outside of the region of homology between the donor template and targeted genomic region. The PCR products were purified and digested with AgeI restriction enzyme. To further determine the OTC Intron 4 on-target site, the genomic region was amplified by nested PCR. Briefly, the genomic DNA was first amplified by the HDR-Fwd and HDR-Rev primers (Table 3) using Q5® High-Fidelity DNA Polymerase (New England Biolabs) and gel purified to remove the residual AAV8.sgRNA1.donor in the genomic DNA. Then nested PCR was performed by using the purified 1st round PCR amplicon. Libraries were made from 250 ng of the 2nd PCR products using NEBNext® Ultra™ DNA Library Prep Kit for Illumina (NEB) and sequenced on Illumina MiSeq (2×250 base pair (bp) paired end or 2×300 bp paired end, Genewiz). Data were processed according to standard Illumina sequencing analysis procedures. Processed reads were mapped to the PCR amplicons as reference sequences using Burrows-Wheeler Aligner with custom scripts. Reads that did not map to reference were discarded. Insertions and/or deletions were determined by comparison of reads against reference using custom scripts. The most likely off-target sites were determined using the algorithm described in www.benchling.com, referred to as OT1 through OT16 (Table 4 below). Primers spanning these sites (see Table immediately following) were used to amplify relevant sequences by nested PCR. Purified PCR fragments were then subjected to deep sequencing as described above.

TABLE 4
PCR primer sequences for detecting potential off-
target effects by deep sequencing.

| | | SEQ | |
|---|---|---|---|
| | | ID | |
| Primer Name | Sequence (5′→3′) | NO: | Note |
| OTC_OT1F1 | CTGGTGCCTTTTTCTATCGCC | 37 | Primers |
| OTC_OT1R1 | CCAAGAGCAACTACAATGGCTT | 38 | for OT1 |
| OTC_OT1F2 | GCATTTTCATGAGCATTCCA | 39 | |
| OTC_OT1R2 | CATGTTGTGCCTGCATCTCT | 40 | |
| OTC_OT2F1 | GCAGACTCCAAGATGCAAGAC | 41 | Primers |
| OTC_OT2R1 | GATGTTGTTCCACCCGCATCT | 42 | for OT2 |
| OTC_OT2F2 | CACTGAGCCAAGTCACTGGA | 43 | |
| OTC_OT2R2 | AGGGACAAAACCAAACAGCA | 44 | |
| OTC_OT3F1 | TGGCCTTCTAAAGCAACCAA | 45 | Primers |
| OTC_OT3R1 | CCGTCTCCCAGATCACATGAC | 46 | for OT3 |
| OTC_OT3F2 | ATAACTCATAATCTATGCATGGCA | 47 | |
| | CAA | | |
| OTC_OT3R2 | TTTGATCATGGTGTTTATCAGAGC | 48 | |
| OTC_OT4F1 | GTCCCCGACAAACCAAGCTA | 49 | Primers |
| OTC_OT4R1 | TGAACTGGCAGTATGCAGGG | 50 | for OT4 |
| OTC_OT4F2 | AACATGGTTTCTGCCCTCAG | 51 | |
| OTC_OT4R2 | GGACCATGCCGAACTCTTAC | 52 | |
| OTC_OT5F1 | TTGAGACCTAGCTCATGCCC | 53 | Primers |
| OTC_OT5R1 | TAACGCAGAACTGGCACAGG | 54 | for OT5 |
| OTC_OT5F2 | CTCTCCCATGGAGAAAGCTG | 55 | |
| OTC_OT5R2 | GAATGTGGCATTGGCTTTTT | 56 | |
| OTC_OT6F1 | GAGAGAGCCAATCTGCCCAT | 57 | Primers |
| OTC_OT6R1 | CACCGGAAACGTGTGAGAGA | 58 | for OT6 |
| OTC_OT6F2 | ACTTCCCATGATCCCATTGA | 59 | |
| OTC_OT6R2 | AGCTTCCCTCCAAGTGTCCT | 60 | |
| OTC_OT7F1 | GATGGGCATAAGCCCGAAGTA | 61 | Primers |
| OTC_OT7R1 | TAAGGCCCAGTGTTGTTGTGT | 62 | for OT7 |
| OTC_OT7F2 | GTAGCAGGGGCTCTGTGAAG | 63 | |
| OTC_OT7R2 | TGGCCTGAAATACCCAGAAC | 64 | |
| OTC_OT8F1 | GTTGAATTCGCGTGTCCAGG | 65 | Primers |
| OTC_OT8R1 | TCCCATGGCGAGAATGTCAC | 66 | for OT8 |
| OTC_OT8F2 | CCCTGTAGGAAACACAGAGGA | 67 | |
| OTC_OT8R2 | TGCTTTGGATGTTGATTCTAAA | 68 | |
| OTC_OT9F1 | GGCAAAGGACTAGCTTGCAC | 69 | Primers |
| OTC_OT9R1 | GGGTGCTATGAGGACCAGTG | 70 | for OT9 |
| OTC_OT9F2 | CAGTGGTGTGTGGAGAGCTG | 71 | |
| OTC_OT9R2 | AGAGAGAGCGCTTGACTTTGA | 72 | |
| OTC_OT10F1 | CTTTGACTCCCGGCGAAAGA | 73 | Primers |
| OTC_OT10R1 | TTGTCCATCCGGGTCATTGC | 74 | for |
| OTC_OT10F2 | AAGTCCTTCTTGCCCAACTT | 75 | OT10 |
| OTC_OT10R2 | CAGCCCCAATGCATTTTT | 76 | |
| OTC_OT11F1 | ATTACAGGTCCTGGTTGGGC | 77 | Primers |
| OTC_OT11R1 | ACTGAGCCTGGTAGAGCCTT | 78 | for |
| OTC_OT11F2 | GGAAGGTGAAGGAAGGAAGG | 79 | OT11 |
| OTC_OT11R2 | TTTTCTAGGAATTCAGGACATACA | 80 | |
| OTC_OT12F | TCATGGTCCTTAAAATTTTTGC | 81 | Primers |
| OTC_OT12R | TCCAGGTATGCAAAGTGGAT | 82 | for |
| | | | OT12 |
| OTC_OT13F1 | TTCAGTTGTACTTTGGATGCTCTGA | 83 | Primers |
| OTC_OT13R1 | CATCTGAATAGCAGCAGGCG | 84 | for |
| OTC_OT13F2 | TAGCACAGCCCAAATGACTT | 85 | OT13 |
| OTC_OT13R2 | TCATGAAACCCCATAATCAGAA | 86 | |
| OTC_OT14F1 | AGTGGGTCATCCTTTGTTACCC | 87 | Primers |
| OTC_OT14R1 | TGCCAGTTATCAGCCAAGCA | 88 | for |
| OTC_OT14F2 | CCCAGGAACTTAACTCAGGTG | 89 | OT14 |
| OTC_OT14R2 | TGCCATTTGACCTCATAAGTCT | 90 | |
| OTC_OT15F1 | TTCAGCCCCCTTGAGTGTTTA | 91 | Primers |
| OTC_OT15R1 | GTCTCTGAGCACAAAGAGACGA | 92 | for |
| OTC_OT15F2 | TTGCCTGTCCCAACTAGAGC | 93 | OT15 |
| OTC_OT15R2 | GGCCCAAGAATGCACATTTA | 94 | |
| OTC_OT16F1 | CCACACACTGGCTAGGACTG | 95 | Primers |
| OTC_OT16R1 | ACTGGCAGCACTTGAGACAA | 96 | for |
| OTC_OT16F2 | GATGGCATGCTGTGGTTTTT | 97 | OT16 |
| OTC_OT16R2 | CAATGCTTCCACACAGAACC | 98 | |

Frequencies of on-target and off-target indels and on-target correction of the spf^{ash} mutation were determined as follows. MiSeq reads were analyzed using custom scripts to identify indels by matching reads against reference, with indels involving any portion of the sequence within 15 nt upstream or downstream of the predicted CRISPR-Cas9 cleavage site (3 nt downstream of the PAM, within the protospacer) considered to be possible off-target effects. Reads for which there was any 18-nt sequence with more than 2 mismatches with the corresponding 18-nt portion of the reference sequence, either upstream or downstream of a candidate indel, were discarded as errors. All candidate indels for the OT1 through OT16 sites were manually curated for confirmation. For the OTC intron 4 on-target site, a read was counted as having “Perfect HDR” if on the antisense strand there was a perfect match with a 51-nt sequence from the donor, starting with the donor-specific ‘CACCAA’ at the location of the PAM, through the donor-specific AgeI insert ‘ACCGGT’, and ending with the SNV ‘C’ at the spf^{ash} OTC mutation site. A read was counted as being a “Read with a ‘G’” if it either (1) met the criterion for “Perfect HDR” or (2) had the SNV ‘G’ on the sense strand in the expected spf^{ash} OTC mutation site 54 nt upstream of the predicted CRISPR-Cas9 cleavage site (accounting for the size of the donor-specific AgeI insert ‘ACCGGT’), with up to two mismatches with the 18-nt intronic portion of the reference sequence adjacent to the spf^{ash} OTC mutation site. A read was counted as having “Partial HDR” if it did not meet the criteria for “Perfect HDR” and “Read with a ‘G’” and if there was a perfect match with an 18-nt sequence from the donor, starting with the donor-specific ‘CACCAA’ at the 3′ end of the target site and ending with the donor-specific AgeI insert ‘ACCGGT’.

L. Statistical Analysis

Test and control vectors were evaluated in at least 3 mice per group at each time point to ensure reproducibility. Sample sizes are noted in figure legends. All animals with successful temporal vein injection were included in the study analysis. Those with unsuccessful injection were excluded. Injection success was determined according to vector genome copies in liver via qPCR, where animals with vector genome copies <10% of the mean value of the dosing group at the same time point were considered to be unsuccessful.

Statistical analysis was performed with GraphPad Prism 6.03 for Windows. The Dunn's multiple comparisons test was used to compare a number of variables with a single control. The Mann-Whitney test was used to determine differences between two groups. The Mantel-Cox test was used to test the survival distributions for differences. Group averages were presented as mean±S.E.M.

M. RFLP Knock-in and Targeting Assays

Genomic DNA of MC57G cells co-transfected with pX330 and OTC donor template was extracted using the QuickExtract™ DNA extraction solution (Epicentre Biotechnologies). RFLP assays were performed to detect homology direct repair (HDR). Briefly, the genomic DNA was amplified by using the HDR-Fwd and HDR-Rev primers. The HDR-Fwd and HDR-Rev primers are designed to anneal outside of the region of homology between the OTC donor template and targeted genomic region. The PCR products (30 cycles, 67° C. annealing and 1 min extension at 72° C.) were purified by QIAQuick PCR purification kit (Qiagen) and digested with AgeI. The digested product was loaded on a 4-20% gradient polyacrylamide. TBE gel and stained with SYBR Gold dye. The cleavage products was imaged and quantified as described above for the SURVEYOR® assay. All PCR reactions were performed using Phusion® High-fidelity DNA polymerase (New England BioLabs) in conjunction with HF Buffer and 3% dimethylsulphoxide.

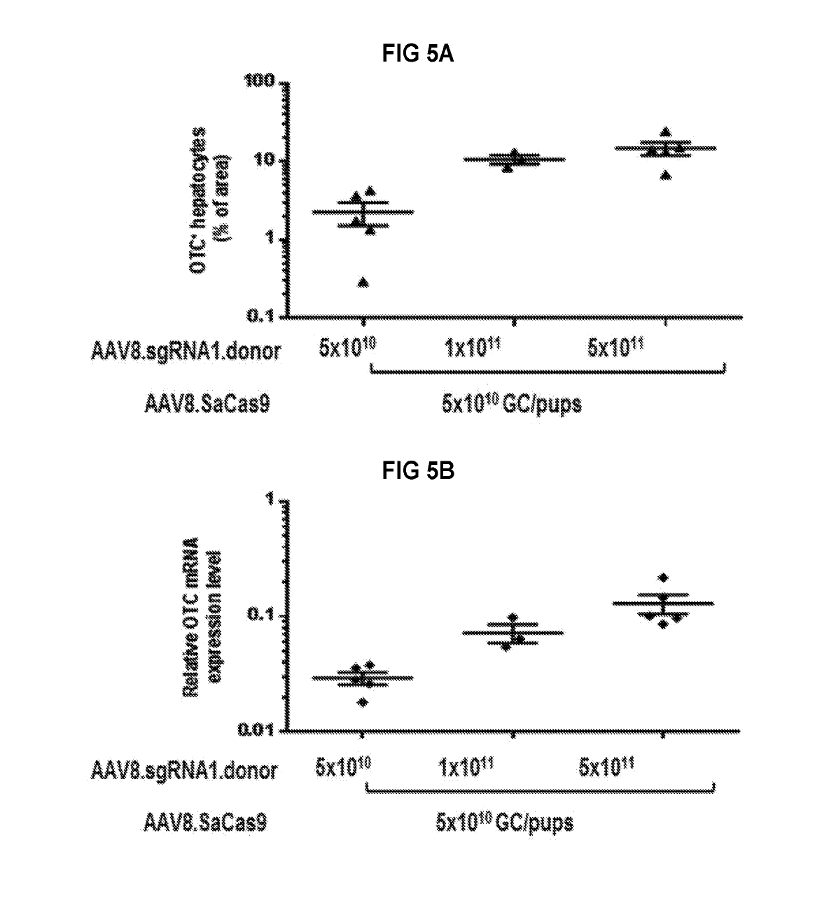

A complete analysis of both the SpCas9 and SaCas9 vector systems in spf^{ash} animals in which samples were obtained 1, 3 and 8 weeks following vector infusion was performed. Controls included samples from wild type litter mates, and spf^{ash} mice that were either untreated or administered AAV8.Cas9 and AAV8.donor without guide RNA (called untargeted). Untreated and untargeted spf^{ash} animals are referred to as spf^{ash} controls, while spf^{ash} mice infused with AAV8.SaCas9 plus AAV8.sgRNA1.donor are referred to as treated animals. Pilot experiments elucidated optimal conditions of vector infusion with respect to doses and ratios of the two vectors (FIG. 5); all subsequent experiments in newborns were conducted with 5×10¹¹ GC of AAV8.sgRNA1.donor (or AAV8.control.donor) and 5×10¹⁰ GC of AAV8.SaCas9.

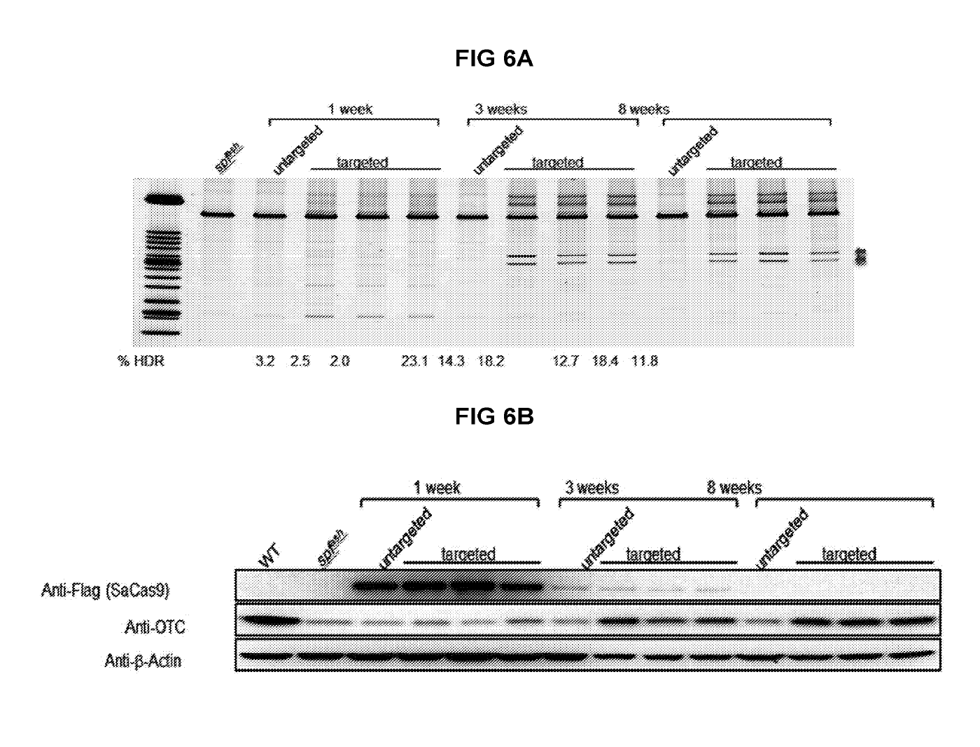

To assess in vivo indel formation and HDR, liver DNAs were recovered from mice at 3 weeks (n=3) and 8 weeks (n=3) after neonatal vector treatment, followed by amplification of the OTC target region by nested PCR and deep sequencing; similar analyses were conducted on DNAs from an untreated spf^{ash} mouse to assess background due to sequencing errors. Table 5 below summarizes the data. Following gene correction, indels were detected in 31% (26.5%-35.5%) of OTC alleles from the 6 treated animals (Table 5). More detailed studies in two treated mice indicated that over 90% of the deletions were less than 20 bp and only 1% extended into the adjacent exon. HDR-based correction of the G-to-A mutation was observed in 10% (6.7%-20.1%) of OTC alleles from 6 treated animals. Analysis of amplified DNA between the G-to-A mutation and the donor-specific, altered PAM located 51 nt into the adjacent intron showed that approximately 83% of corrected alleles contained only donor derived-sequences between these two landmarks (reads with perfect HDR), while 3.5% of total OTC alleles had evidence of incomplete HDR events (reads with partial HDR). HDR-mediated targeted modifications were also estimated by the presence of a restriction-fragment length polymorphism (RFLP) introduced into the donor DNA in three animals harvested at each of the three time points. The average rate of HDR was 2.6% at 1 week, 18.5% at 3 weeks, and 14.3% at 8 weeks, confirming the high rate of HDR observed by deep sequencing (FIG. 6A).

TABLE 5
Summary of the frequencies of indels, correction of OTC spf^{ash} mutation,
and HDR in animals treated with the dual AAV gene-targeting vectors.

| | | | | OTC | | |
|---|---|---|---|---|---|---|
| | | Time after | | reads with | Reads with | Reads with |
| | | treatment | Indel | a ‘G’ | Perfect HDR | Partial HDR |
| ID | Treatment | (week) | (%) | (%) | (%) | (%) |
| 4001 | Neonatal, sgRNA1 | 3 | 26.5 | 20.1 | 17.2 | 7.6 |
| 4003 | Neonatal, sgRNA1 | 3 | 35.5 | 7.2 | 6.4 | 2.7 |
| 307 | Neonatal, sgRNA1 | 3 | 30.5 | 8.9 | 7.2 | 2.9 |
| 835 | Neonatal, sgRNA1 | 8 | 26.7 | 6.8 | 5.5 | 2.2 |
| 836 | Neonatal, sgRNA1 | 8 | 29.6 | 10.8 | 8.9 | 3.3 |
| 844 | Neonatal, sgRNA1 | 8 | 34.4 | 6.7 | 5.4 | 2.4 |
| 630 | Adult, untargeted, low dose | 3 | 0.3 | 0.02 | 0.01 | 0.01 |
| 637 | Adult, sgRNA1, low dose | 3 | 50.3 | 0.3 | 0.2 | 0.1 |
| 640 | Adult, sgRNA1, low dose | 3 | 45.0 | 0.3 | 0.3 | 0.1 |
| 641 | Adult, sgRNA1, low dose | 3 | 38.5 | 0.2 | 0.1 | 0.04 |
| 658 | Adult, untargeted, high dose | 2 | 0.1 | 0.03 | 0.02 | 0.01 |
| 648 | Adult, sgRNA1, high dose | 2 | 43.6 | 1.8 | 1.5 | 0.3 |
| 649 | Adult, sgRNA1, high dose | 2 | 48.5 | 2.1 | 1.7 | 0.4 |
| 653 | Adult, sgRNA1, high dose | 2 | 34.0 | 1.3 | 1.1 | 0.2 |
| | Untreated spf^{ash} | n/a | 0.04 | 0.002 | 0.001 | 0.004 |

The algorithm described in www.benchling.com identified 49 potential off-target sites for sgRNA1; the top 15 sites most likely to create DSBs were amplified by PCR and subjected to deep sequencing (See following Table 6). The background from DNA of untreated animals due to sequencing error varied between sites, although it was usually a fraction of a percent. Samples from treated animals did not demonstrate evidence of indels that were above this background.

TABLE 6
Full list of off-target sites.

| | | SEQ | | | | |
|---|---|---|---|---|---|---|
| | | ID | | | | |
| ID | Sequence | NO: | PAM | Score | On-target | Chromosome |
| sgRNA1 | TCTCTTTTAAACTAACCCAT | 99 | CAGAG | 100.00 | TRUE | X |
| OT1 | 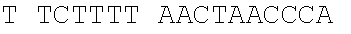 | 100 | TAGGA | 1.14 | FALSE | chr17 |
| 0T2 | 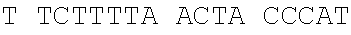 | 101 | AGGGG | 0.86 | FALSE | chrX |
| 0T3 | 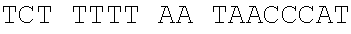 | 102 | TAGAA | 0.86 | FALSE | chr8 |
| 0T4 | 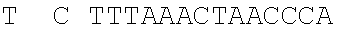 | 103 | CTGGG | 0.85 | FALSE | chr5 |
| OT5 | 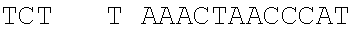 | 104 | CTGAA | 0.83 | FALSE | chr19 |
| 0T6 | 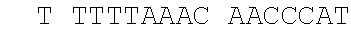 | 105 | CTGGA | 0.66 | FALSE | chr2 |
| 0T7 | 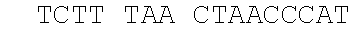 | 106 | GTGAA | 0.63 | FALSE | chr6 |
| 0T8 | 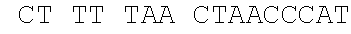 | 107 | GTGAA | 0.62 | FALSE | chr9 |
| 0T9 | 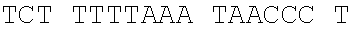 | 108 | CAGGG | 0.59 | FALSE | chr1 |
| OT10 | 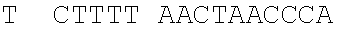 | 109 | GTGGG | 0.54 | FALSE | chr8 |
| OT11 | 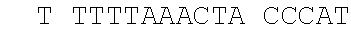 | 110 | GTGAG | 0.48 | FALSE | chr12 |
| OT12 | 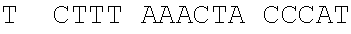 | 111 | CTGAA | 0.48 | FALSE | chrX |
| 0T13 | 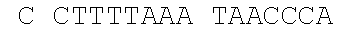 | 112 | ATGAA | 0.47 | FALSE | chr8 |
| 0T14 | 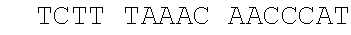 | 113 | TGGAG | 0.46 | FALSE | chr6 |
| OT15 | 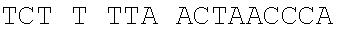 | 114 | CTGAG | 0.46 | FALSE | chrX |
| 0T16 | 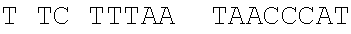 | 115 | ATGGA | 0.46 | FALSE | chr15 |
| 0T17 | 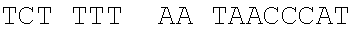 | 116 | GTGAA | 0.46 | FALSE | chr6 |
| 0T18 | 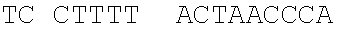 | 117 | CAGAG | 0.46 | FALSE | chr5 |
| 0T19 | 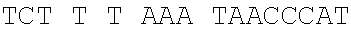 | 118 | CAGAG | 0.46 | FALSE | chr18 |
| 0T20 | 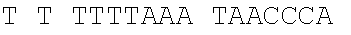 | 119 | ATGAA | 0.45 | FALSE | chr17 |
| 0T21 | 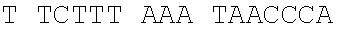 | 120 | AGGGA | 0.43 | FALSE | chr1 |
| 0T22 | 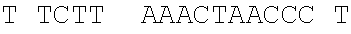 | 121 | GTGGA | 0.42 | FALSE | chr7 |
| 0T23 | 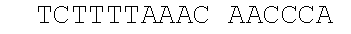 | 122 | AAGGA | 0.39 | FALSE | chr8 |
| 0T24 | 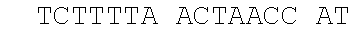 | 123 | AAGAA | 0.39 | FALSE | chr12 |
| 0T25 | 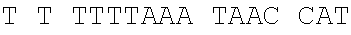 | 124 | TGGGG | 0.38 | FALSE | chr6 |
| 0T26 | 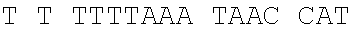 | 125 | TGGGA | 0.38 | FALSE | chr15 |
| 0T27 | 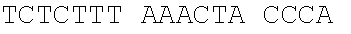 | 126 | GGGAA | 0.37 | FALSE | chr11 |
| 0T28 | 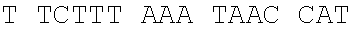 | 127 | GAGAA | 0.36 | FALSE | chrX |
| 0T29 | 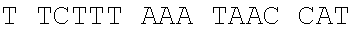 | 128 | GAGGG | 0.36 | FALSE | chr8 |
| 0T30 | 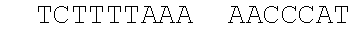 | 129 | AAGAA | 0.35 | FALSE | chr12 |
| 0T31 | 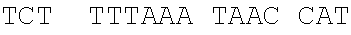 | 130 | TTGGA | 0.35 | FALSE | chr8 |
| 0T32 | 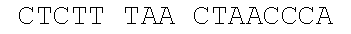 | 131 | TGGGA | 0.35 | FALSE | chr6 |
| 0T33 | 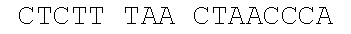 | 132 | ATGGA | 0.35 | FALSE | chr3 |
| 0T34 | 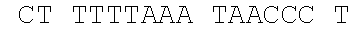 | 133 | TTGGA | 0.34 | FALSE | chr9 |
| 0T35 | 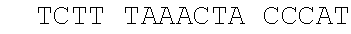 | 134 | TAGAA | 0.34 | FALSE | chr19 |
| 0T36 | 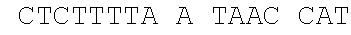 | 135 | TAGAA | 0.34 | FALSE | chr18 |
| 0T37 | 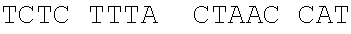 | 136 | AAGAG | 0.33 | FALSE | chr19 |
| 0T38 | 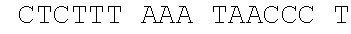 | 137 | TAGAA | 0.33 | FALSE | chr2 |
| 0T39 | 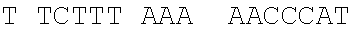 | 138 | GGGAA | 0.32 | FALSE | chr2 |
| 0T40 | 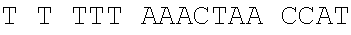 | 139 | CTGAA | 0.32 | FALSE | chr13 |
| 0T41 | 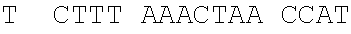 | 140 | GTGAA | 0.32 | FALSE | chr11 |
| 0T42 | 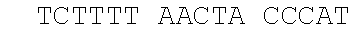 | 141 | TTGGA | 0.31 | FALSE | chr17 |
| 0T43 | 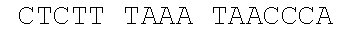 | 142 | AAGAA | 0.31 | FALSE | chr14 |
| 0T44 | 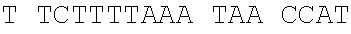 | 143 | CTGAG | 0.31 | FALSE | chr10 |
| 0T45 | 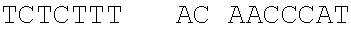 | 144 | GTGAG | 0.31 | FALSE | chr2 |
| 0T46 | 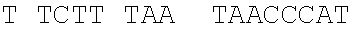 | 145 | TTGAG | 0.31 | FALSE | chr3 |
| 0T47 | 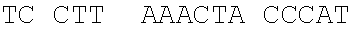 | 146 | AAGGG | 0.30 | FALSE | chr9 |
| 0T48 | 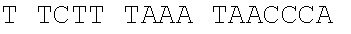 | 147 | TGGGA | 0.30 | FALSE | chr14 |
| 0T49 | 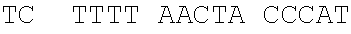 | 148 | TTGAG | 0.28 | FALSE | chr1 |

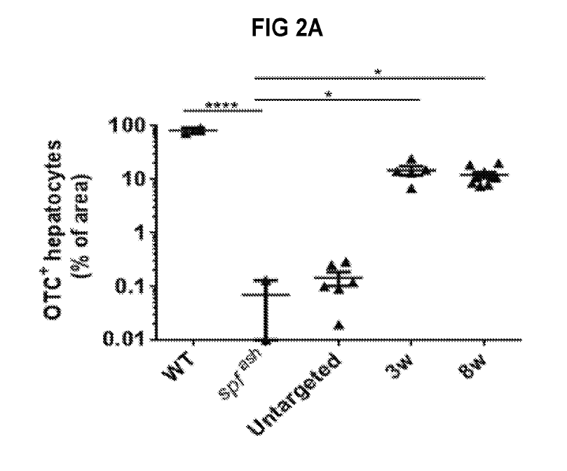

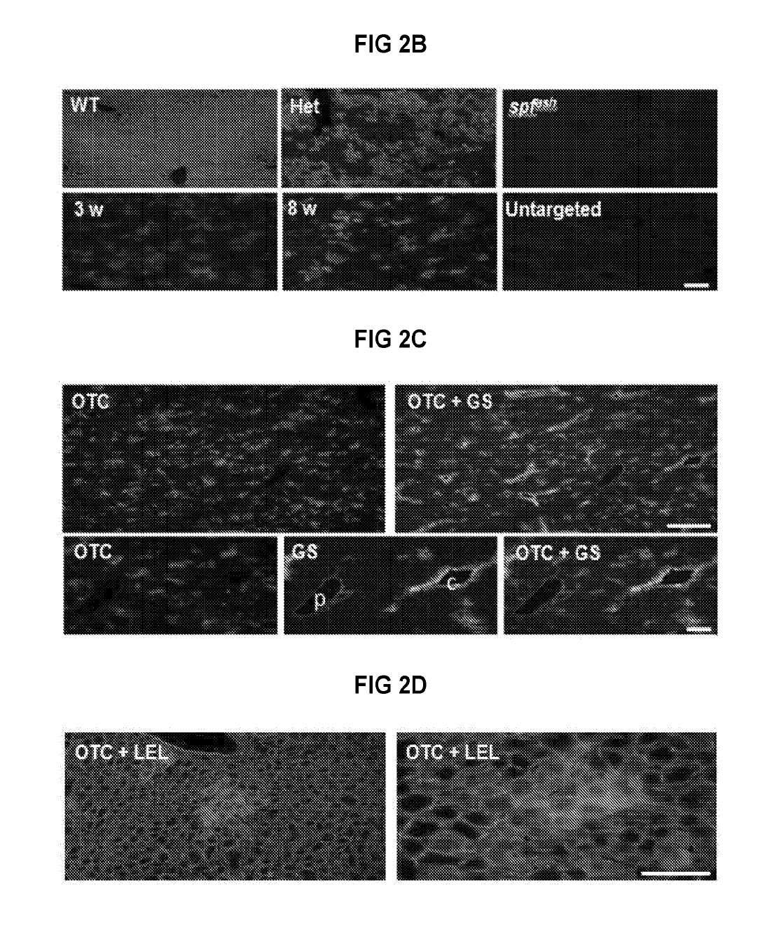

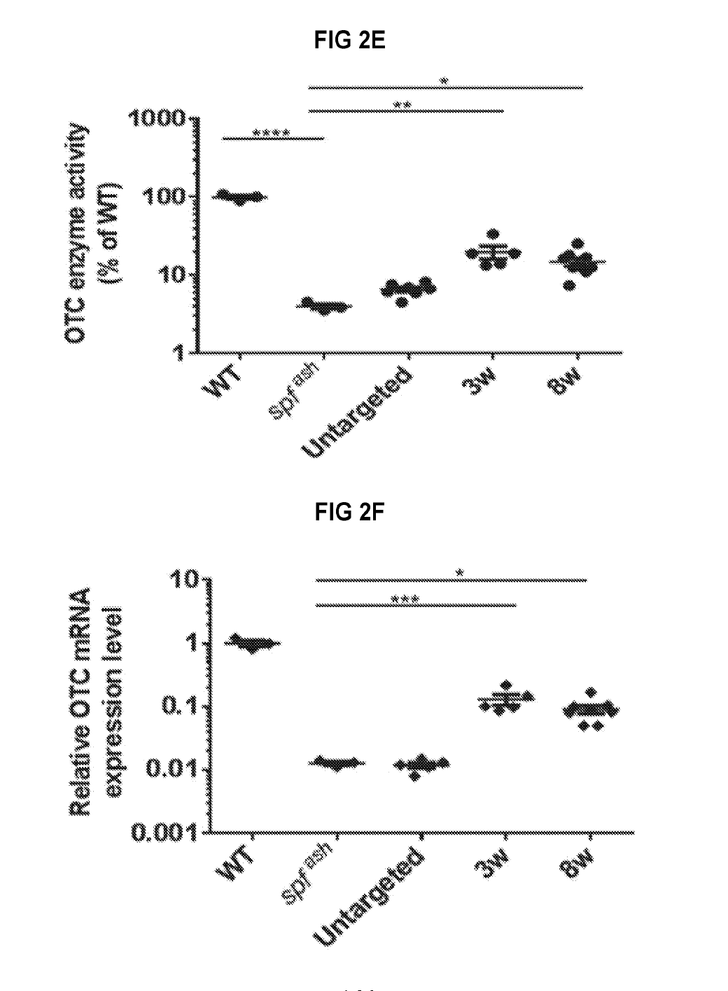

Liver homogenates were evaluated for expression of OTC by Western blot. OTC protein in most treated animals was higher than in spf^{ash} controls, but did not reach the level found in wild type mice (FIG. 6B). Tissue sections of liver were analyzed by immunohistochemistry for OTC expression (FIG. 2C-2E). FIG. 2C shows representative fluorescent micrographs of OTC expression, which were quantified in FIG. 2B for % correction using morphometric analyses. No signal (<1%) was observed in the spf^{ash} controls, while analysis of heterozygotes showed the predicted mosaicism (FIG. 2C). Morphometry indicated 10 to 100 fold higher numbers of OTC expressing cells in treated groups than found in the spf^{ash} control groups (FIG. 2B; 15% (6.8%-24.4% at 3 weeks and 13% (7.5%-20.1% at 8 weeks). Treated animals showed patches of OTC expressing cells (FIG. 2D) that localized within all portions of the portal axis except around central veins, which was visualized by staining for glutamine synthetase. This is the predicted distribution of endogenous OTC [MA Dingemanse, et al, Hepatology, 24: 407-411 (1996)]. A higher magnification histological view demonstrated clusters of OTC expressing hepatocytes consistent with correction followed by clonal expansion in the context of the growing liver (FIG. 2E). Direct measurements of OTC enzyme activity from liver homogenates and OTC mRNA from total cellular RNA from liver revealed similarly high levels of correction in treated animals, resulting in 20% (13.4%-33.7% (n=5) and 16% (11.0%-25.4%; n=8) normal OTC activity at 3 and 8 weeks respectively (FIG. 2F), and 13% (8.6%-21.8%, n=5) and 9% (5.0%-16.8% n=8) of normal OTC mRNA at 3 and 8 weeks (FIG. 2G). Despite the decrease in OTC⁺ hepatocytes, OTC enzyme activity, and OTC mRNA expression from 3 to 8 weeks, none of these differences were statistically significant (FIG. 2b; p=0.4828, FIG. 2e; p=0.2723, FIG. 2f; p=0.1475, respectively). Liver homogenates were also evaluated for expression of OTC by Western blot. OTC protein levels in most treated animals were higher than in spf^{ash} controls but did not reach the levels found in wild-type mice. Overall, there was good correlation in the estimates of correction based on histology, protein, and mRNA within individual animals.

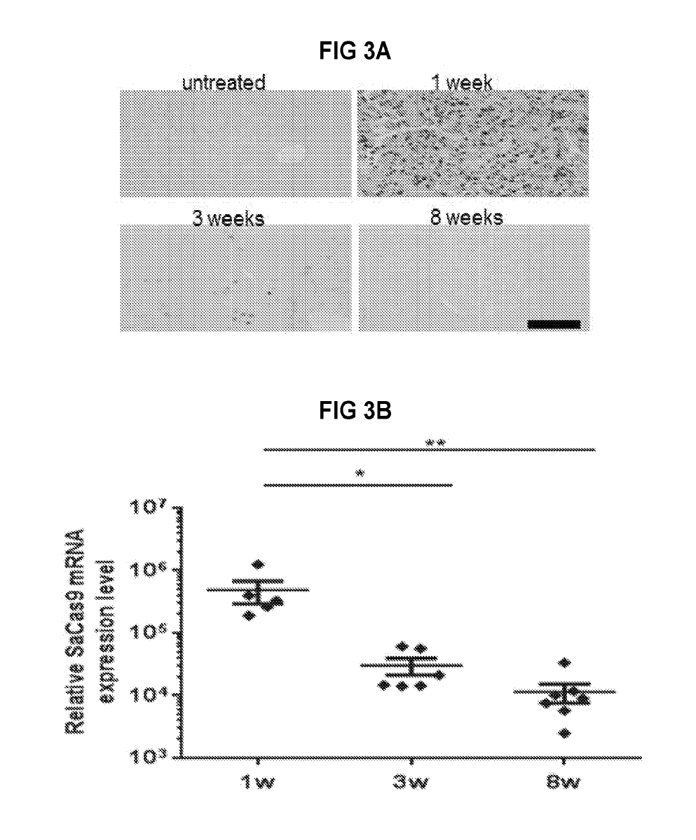

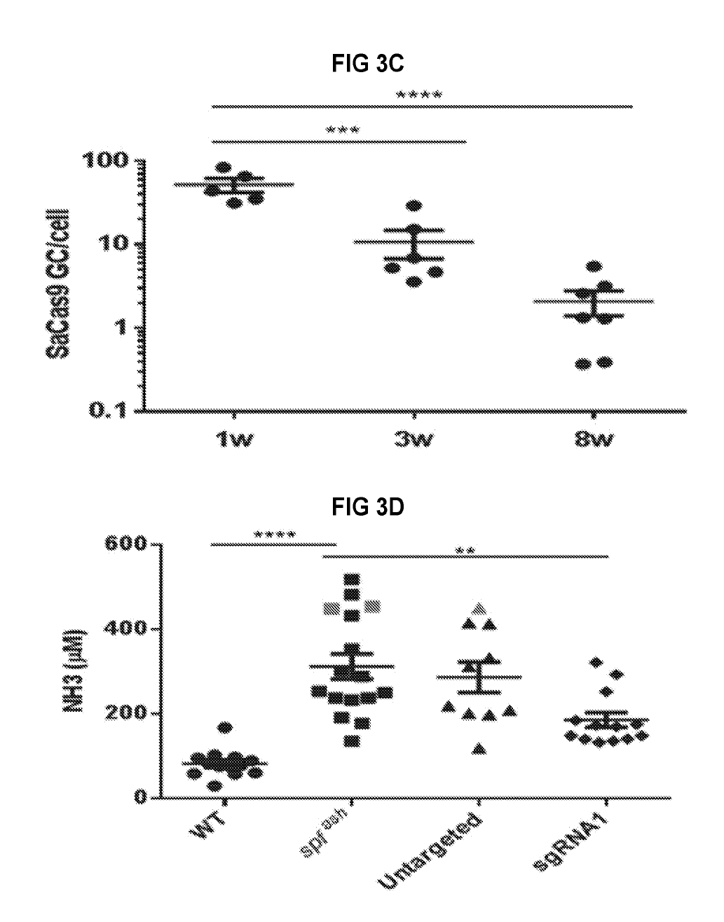

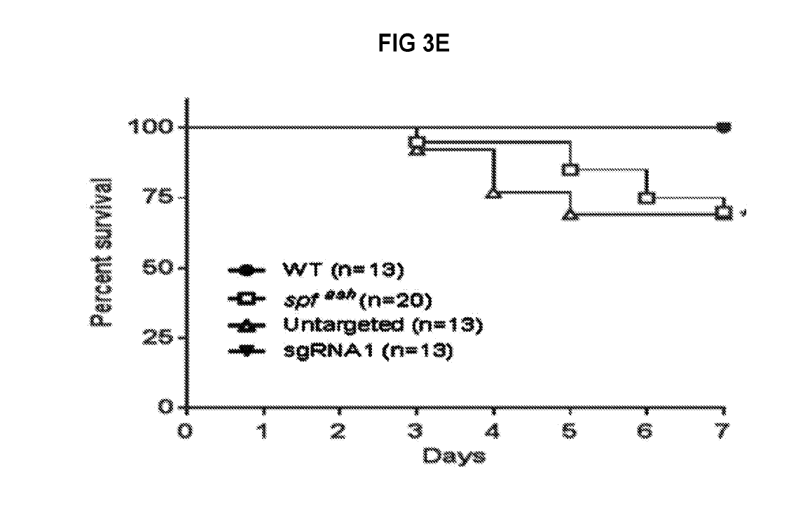

One concern about using AAV to deliver SaCas9 is its propensity to achieve stable transgene expression which is not necessary to accomplish editing and may in fact contribute to immune and/or genome toxicity. Western blot analysis showed high level SaCas9 protein at 1 week that declined to undetectable levels by 8 weeks (FIG. 6B). Furthermore, immunohistochemistry revealed nuclear-localized SaCas9 protein in 21% of hepatocytes at one week, which declined to undetectable levels (<0.1% hepatocytes) by 8 weeks (FIG. 3A). SaCas9 mRNA declined 43-fold during this 8 week period, to very low but still detectable levels (FIG. 3B). A 25-fold reduction in SaCas9 DNA during this same time interval indicates that elimination of vector genomes in the setting of the proliferating newborn liver is a primary contributor to the desired decline of SaCas9 expression (FIG. 3C).

In assessing the impact of gene correction on the clinical manifestations of OTC deficiency, we evaluated the tolerance of spf^{ash} mice to a one-week course of a high protein diet. As expected, measurement of blood ammonia at the end of the diet revealed a statistically significant elevation from 83±9 μM (n=13) in wild type controls to 312±30 (n=16) in the spf^{ash} controls (FIG. 3D; p<0.0001). Substantial variation in blood ammonias were found in untreated spf^{ash} animals after the 7-day high protein diet, which is consistent with findings in OTC deficient patients who show large fluctuations in ammonia over relatively short periods of time [D. Moscioni et al, Mol Ther, 14: 25-33 (2006)]. There was no significant difference between untreated spf^{ash} and untargeted spf^{ash} controls (FIG. 3D; p=0.83); conversely, a statistically significant 40% reduction in ammonia was observed between untreated spf^{ash} and treated spf^{ash} animals (FIG. 3D; p=0.0014). Importantly, treated spf^{ash} mice also demonstrated a statistically significant improvement in survival over untreated or untargeted spf^{ash} (FIG. 3E; p=0.03). During the course of the protein diet, 30% of both untreated spf^{ash} (n=20) and untargeted spf^{ash} (n=13) developed clinical signs of hyperammonia and died or had to be euthanized, while all of the wild type mice (n=13) and treated spf^{ash} mice (n=13) survived (FIG. 3E).

The surprisingly high level of correction in this study is likely due to high expression of SaCas9 with abundant donor DNA in the context of dividing cells. Infusion of AAV8 vectors into newborn monkeys demonstrated the same high peak levels of transduction and gene transfer (i.e., 92% hepatocytes expressing lacZ and 32 vector genomes per cell, respectively) as achieved in these murine studies (i.e., 21% SaCas9-expressing hepatocytes and 52 vector genomes per cell, respectively) with similar kinetics of decline, which is encouraging in terms of translation to larger species including humans.

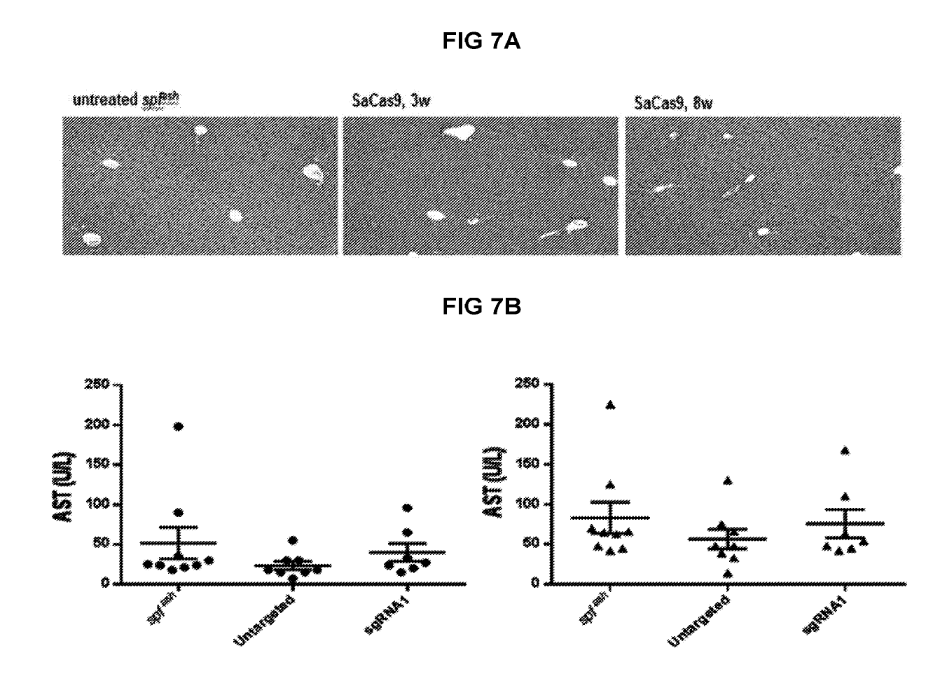

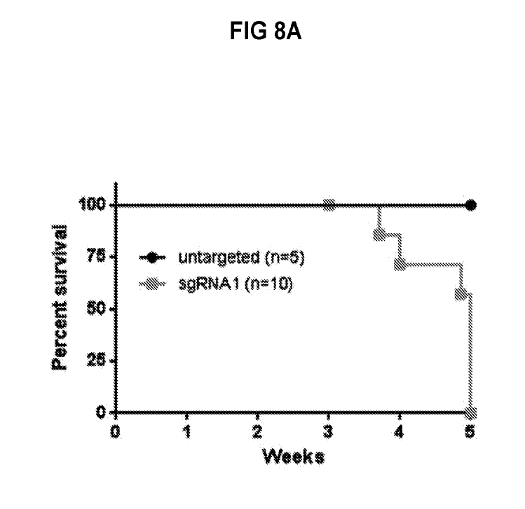

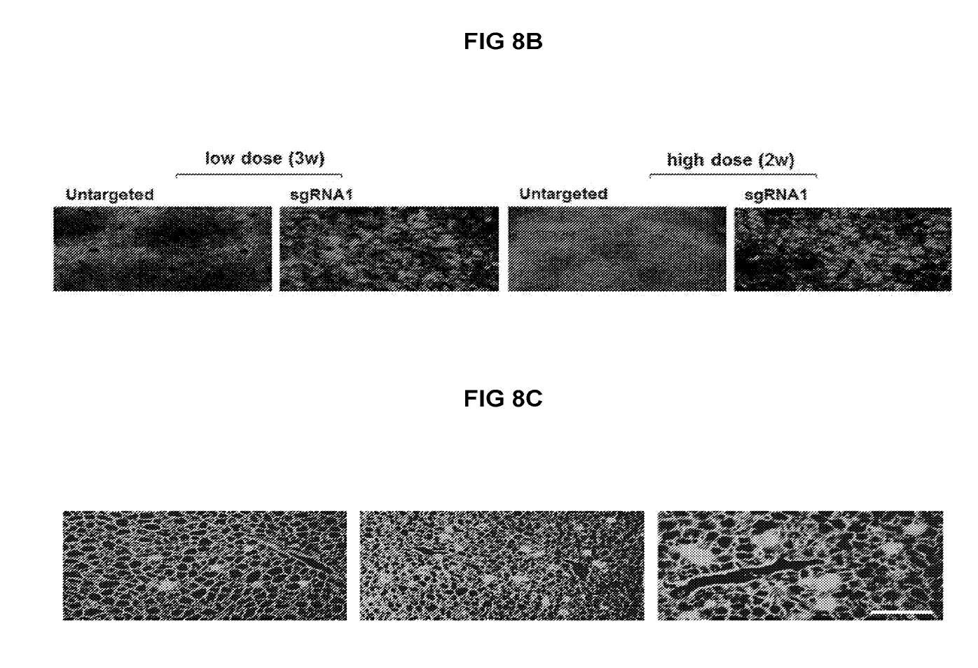

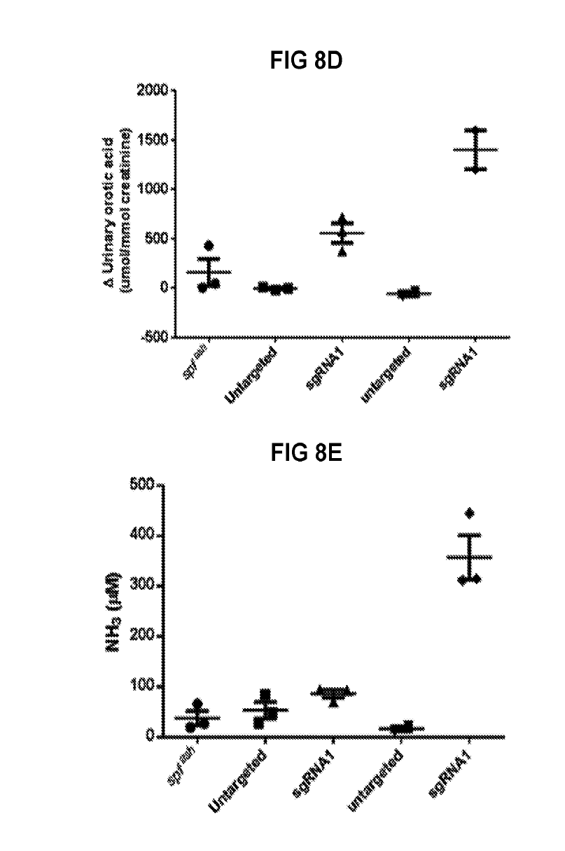

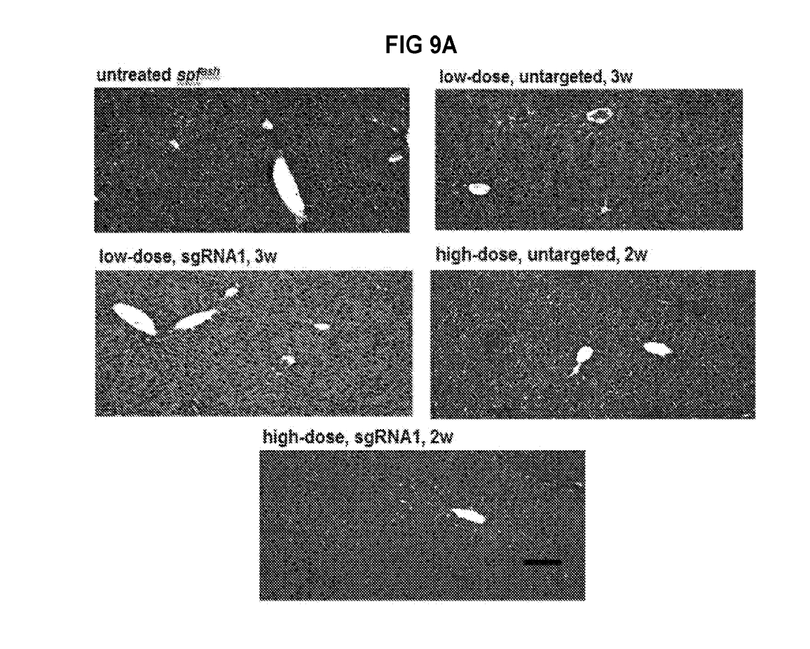

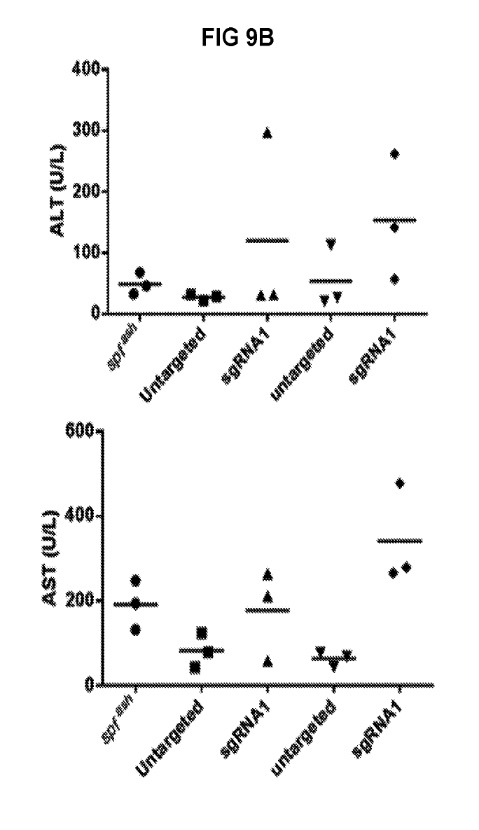

Issues of safety relate primarily to the expression of Cas9 in the context of an sgRNA that could create off-target DSBs with carcinogenic sequelae. More extensive characterization of these potential toxicities is necessary before clinical translation can be considered, although we could not detect off-target indels at the level of sensitivity achieved by deep sequencing of likely off-target sites. Cas9 could also elicit pathologic immune responses, as has been observed in gene replacement therapies in which the transgene is a foreign protein. However, systemic delivery of AAV in a newborn helps mitigate potential immunologic adverse events for several reasons. First, expression of the prokaryotic SaCas9 protein is transient because the non-integrated vector is lost during hepatocyte proliferation. Furthermore, exposure of newborn rhesus macaques to AAV-encoded proteins has been shown to induce tolerance to these proteins thereby circumventing toxicity caused by destructive adaptive immune responses. Detailed histological analyses of liver and transaminase levels in SaCas9-treated spf^{ash} mice harvested at the end of the high protein diet challenge failed to reveal any pathology or toxicity (FIG. 7).

### Example 2—MPSI

A. MPSI Mouse

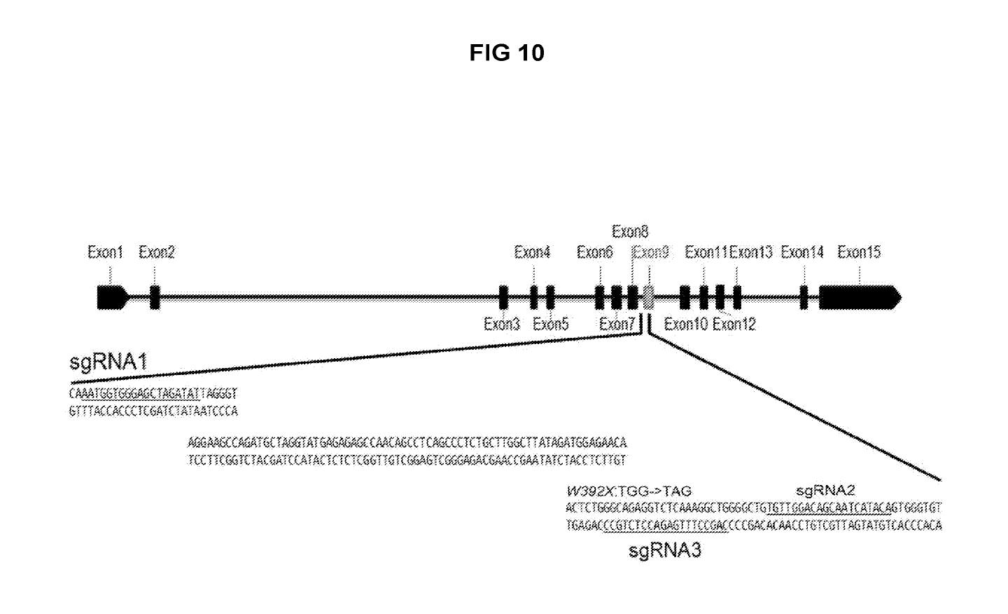

For use in this study, PAM sequences (NNGRRT) in proximity to the MPSI W392X mutation of the MPSI gene were identified as potential 20-nt protospacer sequences. Three sequences, sgRNA1-3 (FIG. 10), were further evaluated following transfection of puromycin-containing plasmids into a mouse MC57G cell line. Low transfection efficiency in this cell line required enrichment of transfected cells by a brief exposure to puromycin. Evidence for double-strand breaks (DSBs) and the formation of indels at the desired site was demonstrated using the SURVEYOR assay.

The AAV donor plasmids are constructed by cloning in the sgRNA under the control of U6 promoter and respective donor template with approximately 1 kb of sequence flanking each side of the mutation and the corresponding PAM sequence in the donor template is mutated to reduce re-cleavage after HDR.

Initial in vivo assessment was performed in neonatal MPSWX pups targeting the liver by co-injection of AAV8 vector expressing SaCas9 under the control of liver-specific TBG promoter (AAV8.TBG.SaCas9) and AAV8.sgRNA.donor. Eight weeks after vector treatment, liver DNA were isolated for indel analysis by Surveyor assay and high frequency of indels were generated by both sgRNA2 and sgRNA3.

Western blot on tissue lysate from wild type and MPSWX mouse showed that brain is the dominant tissue that expresses IDUA enzyme as compared to heart, liver, spleen and kidney. For in vivo gene correction, in order to target brain, AAV9 vector expressing SaCas9 under the control of an ubiquitous promoter CBh was made. The sgRNA and donor template vectors are also packaged with AAV9 capsid for broad in vivo transduction and crossing the blood-brain barrier. For in vivo assessment, AAV9.SaCas9 and AAV9.sgRNA.donor vectors will be co-injected into neonatal MPSI pups via temporal vein or intracerebroventricular (ICV) delivery. The correction will be characterized in treated mice: biochemistry (IDUA protein and activity), histology (GAG storage), DNA, RNA, and off-target analysis.

B. MPSI Dog

Donor template sequences are provided in the following table.

TABLE 7

| | | SEQ | |
|---|---|---|---|
| | | ID | |
| ID | Sequence(5′→3′) | NO: | Note |
| dIDUA_sgRNA1_ | CACCGTTCTGATGAGGGCTCCGCGG | 149 | hSpCas9 |
| Fwd | | | |
| dIDUA_sgRNA1_ | AAACCCGCGGAGCCCTCATCAGAAC | 150 | |
| Rev | | | |
| dIDUA_sgRNA2_ | CACCGCACCTGGTGCTCGTGGACG | 151 | |
| Fwd | | | |
| dIDUA_sgRNA2_ | AAACCGTCCACGAGCACCAGGTGC | 152 | |
| Rev | | | |
| dIDUA_sgRNA3_ | CACCGGGCCGCGTCCACGAGCACC | 153 | |
| Fwd | | | |
| dIDUA_sgRNA3_ | AAACGGTGCTCGTGGACGCGGCCC | 154 | |
| Rev | | | |
| dIDUA_sgRNA4_ | CACCGTCCACGAGCACCAGGTGCG | 155 | |
| Fwd | | | |
| dIDUA_sgRNA4_ | AAACCGCACCTGGTGCTCGTGGAC | 156 | |
| Rev | | | |
| dIDUA_sgRNA5_ | CACCGTGGACGCGGCCCGCGCGCTG | 157 | |
| Fwd | | | |
| dIDUA_sgRNA5_ | AAACCAGCGCGCGGGCCGCGTCCAC | 158 | |
| Rev | | | |
| dIDUA_sgRNA6_ | CACCGCTTCTGATGAGGGCTCCGC | 159 | |
| Fwd | | | |
| dIDUA_sgRNA6_ | AAACGCGGAGCCCTCATCAGAAGC | 160 | |
| Rev | | | |

The sgRNA candidates listed in the table above will be validated on MDCK cells (canine cell line). Site-directed mutagenesis will be performed on the PAM sequence on the donor template corresponding to the selected sgRNA. The AAV donor plasmids are constructed by cloning in the sgRNA and respective donor template and used for AAV vector production. The dual AAV vector system is injected into neonatal MPSI puppies. The correction is characterized in the dogs by biochemistry, histology, DNA, RNA, and off-target analysis.

### Example 3—hCFTR—ΔF508 Human Airway Cells

sgRNA for SaCas9 have been selected. The sgRNA primers will be closed into pX330.SaCas9. The donor template will then be PCR cloned.

TABLE 8

| | | SEQ ID | |
|---|---|---|---|
| ID | Sequence(5'->3' | NO: | Note |
| hCFTR_sgRNA1_Fwd | 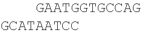 | 161 | hSpCas9 |
| hCFTR_sgRNA1_Rev | 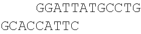 | 162 | |
| hCFTR_sgRNA2_Fwd | 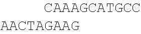 | 163 | |
| hCFTR_sgRNA2_Rev | 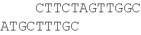 | 164 | |
| hCFTR_sgRNA3_Fwd | 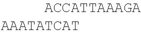 | 165 | |
| hCFTR_sgRNA3_Rev | 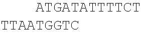 | 166 | |
| hCFTR_sgRNA4_Fwd | 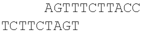 | 167 | |
| hCFTR_sgRNA4_Rev | 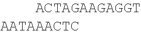 | 168 | |

In order to validate the sgRNAs in vitro, pX330.SaCas9-sgRNA will be transfected into 293 cells. DNA will then be isolated for Indel analysis. The AAV donor plasmids are constructed by cloning in the sgRNA and respective donor template. The resulting plasmids are used to construct AAV vector (AAV6.2). The dual AAV6.2 viral vector system is used for in vitro transduction on ΔF508 human airway cells and the correction in these airway cells is characterized. For in vivo assessment, the testing system is DF508 CFTR mouse. Vectors can be delivered systemically to neonatal mice; or to adult mouse lung. AAV serotypes can be AAV6.2 or AAV9.

### Example 4—Correction of FIX KO Phenotype in Mice

SaCas9-mediated insertion of a therapeutic gene. PAM sequences (NNGRRT) in exon 2 of the murine factor IX gene were identified as potential 20-nt protospacer sequences for use in this study. sgRNA candidates were validated in vitro for Indels following the transfection into mouse cell line and sgRNA3 showed the highest efficiency in indel generation. AAV donor vector contains U6.sgRNA, donor arms, and. hFIXco-Padua.bGH (exon 2-8) were constructed. AAV8 vectors were produced and injected into neonatal FIX-KO pups and adult FIX-KO mice. Expression of Cas9 will be characterized, and the correction of the phenotype will be determined by measuring FIX expression, and performing histological examinations. DNA and RNA species will also be characterized, and off-target analyses will be performed.

TABLE 9

| | | SEQ | |
|---|---|---|---|
| | | ID | |
| ID | Sequence(5′→3′) | NO: | Note |
| mFIXsgRNA1_ | CACCGTTTAGGATATCTA | 169 | mFIX target |
| Fwd | CTCAGTA | | sequence 1 for |
| | | | hSaCas9 |
| mFIXsgRNA1_ | AAACTACTGAGTAGATAT | 170 | |
| Rev | CCTAAAC | | |
| mFIXsgRNA2_ | CACCGACAAACCTGCAC | 171 | mFIX target |
| Fwd | ATTCGGTA | | sequence 2 for |
| | | | hSaCas9 |
| mFIXsgRNA2_ | AAACTACCGAATGTGCA | 172 | |
| Rev | GGTTTGTC | | |
| mFIXsgRNA3_ | CACCGCACCTGAACACC | 173 | mFIX target |
| Fwd | GTCATGG | | sequence 3 for |
| | | | hSaCas9 |
| mFIXsgRNA3_ | AAACCCATGACGGTGTTC | 174 | |
| Rev | AGGTGC | | |
| mFIX_Point | CGAGGGAGATGGACAAC | 175 | FIX PCR |
| M_F | AAT | | primers |
| mFIX_Point | GCACCACAAGCCCTGTA | 176 | |
| M_R | AAT | | |

### Example 5—Insertion of Therapeutic Gene in Rhesus Macaques

sgRNA candidates have been identified and cloned into pX330.SaCas9 plasmid, and donor template is being de-novo synthesized. In vitro validation of the sgRNA candidates will be performed in LLC-MK2 cell line at the optimal transfection conditions. Cells will be transfected with pX330-sgRNA plasmid in the presence of Cas9. DNA will be purified and assessed for the presence of Indels. Based on Indel assessments, preferred sgRNA candidates will be identified and characterized in rhesus macaques. Selected sgRNA will be used for construction of the AAV donor plasmid in which hFIXco-Padua is flanked by HDR arms. Newborn monkeys will be injected systemically using dual AAV system, and gene insertion will be characterized by measuring hFIX expression, and by measuring activated partial thromboplastin time (APPT).

TABLE 10

| | | SEQ | |
|---|---|---|---|
| | | ID | |
| ID | Sequence(5′→3′ | NO: | Note |
| rhFIXsgRNA1_ | CACCGCGTGTGAACATGAT | 177 | rhFIX target |
| Fwd | CATGG | | seqence 1 for |
| | | | hSaCas9 |
| rhFIXsgRNA1_ | AAACCCATGATCATGTTCA | 178 | |
| Rev | CACGC | | |
| rhFIXsgRNA2_ | CACCGTTTAGGATATCTAC | 179 | rhFIX target |
| Fwd | TCAGTG | | seqence 2 for |
| | | | hSaCas9 |
| rhFIXsgRNA2_ | AAACCACTGAGTAGATATC | 180 | |
| Rev | CTAAAC | | |
| rhFIXsgRNA3_ | CACCGACAAACCTGTACAT | 181 | rhFIX target |
| Fwd | TCAGCA | | seqence 3 for |
| | | | hSaCas9 |
| rhFIXsgRNA3_ | AAACTGCTGAATGTACAGG | 182 | |
| Rev | TTTGTC | | |

### Example 6—Gene Splicing Using Dual AAV Cpf1 CRISPR System

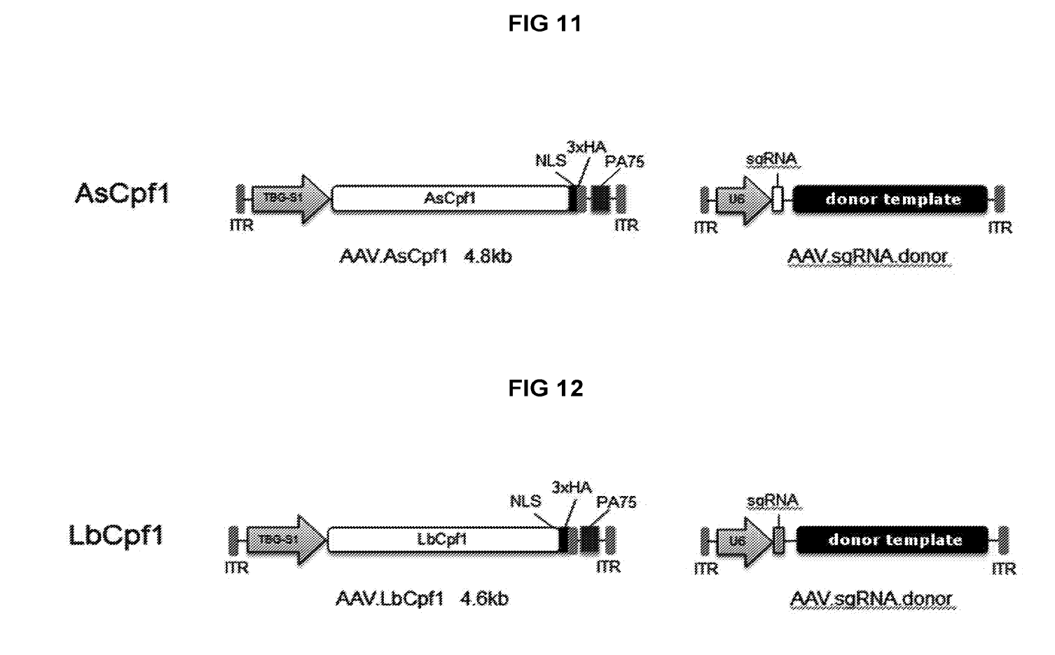

A U6-gRNA scaffold-terminator was cloned into AsCpf1 and LbCpf1 (plasmids containing Cpf1 obtained from Addgene (a non-profit plasmid repository, www.addgene.org), following which the plasmids with DNMT1 target sequences [L. Swiech, et al, Nature biotechnology, 33: 102-106 (2015), e-published 19 Oct. 2014] as a positive control were tested in 293 cells. The Surveyor data showed both constructs functioned as designed. Next, Cpf1 protospacer sequences near the OTC spf^{ash} mutation will be verified in vitro by Surveyor assay. The most efficient sgRNAs will be cloned into pAAV donor plasmids with the corresponding PAM sequence mutated. AAV8 vectors expressing AsCpf1 and LbCpf1 driven by ABP2.TBG-S1 as enhancer/promoter for liver applications (OTC and mFIX-KO as first models) have been generated (FIGS. 11 and 12).

### Example 7—Gene Inactivation Using AAV SaCas9 and PCSK9 sgRNA

Seven PCSK9 sgRNAs that target both monkey and human PCSK9 exons were selected (see Table below) and cloned into pX330.SaCas9 plasmid. Plasmids were transfected into monkey cell line LLC-MK2 and 293 cells. DNA was isolated and target regions were amplified by PCR for Surveyor assay. sgRNA3 showed highest efficiency in both monkey and human cell lines and was selected as the top candidate.

TABLE 11

| | | SEQ | |
|---|---|---|---|
| | | ID | |
| ID | Sequence (5′→3′) | NO: | Note |
| EGFP-sgRNAF | CACCGTCGTGCTGCTTCATGTGGT | 183 | Control |
| EGFP-sgRNAR | AAACACCACATGAAGCAGCACGAC | 184 | |
| PCSK9_sgRNA1F | CACCGATGCTCTGGGCAAAGACAG | 185 | Exon3 |
| PCSK9_sgRNA1R | AAACCTGTCTTTGCCCAGAGCATC | 186 | |
| PCSK9_sgRNA2F | CACCGATCTCCTAGACACCAGCATA | 187 | Exon4 |
| PCSK9_sgRNA2R | AAACTATGCTGGTGTCTAGGAGATC | 188 | |
| PCSK9_sgRNA3F | CACCGAAGTTGGTCCCCAAAGTCCC | 189 | Exon7 |
| PCSK9_sgRNA3R | AAACGGGACTTTGGGGACCAACTTC | 190 | |
| PCSK9_sgRNA4F | CACCGCAGCACCTGCTTTGTGTCA | 191 | Exon7 |
| PCSK9_sgRNA4R | AAACTGACACAAAGCAGGTGCTGC | 192 | |
| PCSK9_sgRNA5F | CACCGAGTCTCTGCCTCAACTCGGC | 193 | Exon8 |
| PCSK9_sgRNA5R | AAACGCCGAGTTGAGGCAGAGACTC | 194 | |
| PCSK9_sgRNA6F | CACCGATGACATCTTTGGCAGAGAA | 195 | Exon8 |
| PCSK9_sgRNA6R | AAACTTCTCTGCCAAAGATGTCATC | 196 | |
| PCSK9_sgRNA7F | CACCGCCTGGTTCCCTGAGGACCA | 197 | Exon8 |
| PCSK9_sgRNA7R | AAACTGGTCCTCAGGGAACCAGGC | 198 | |

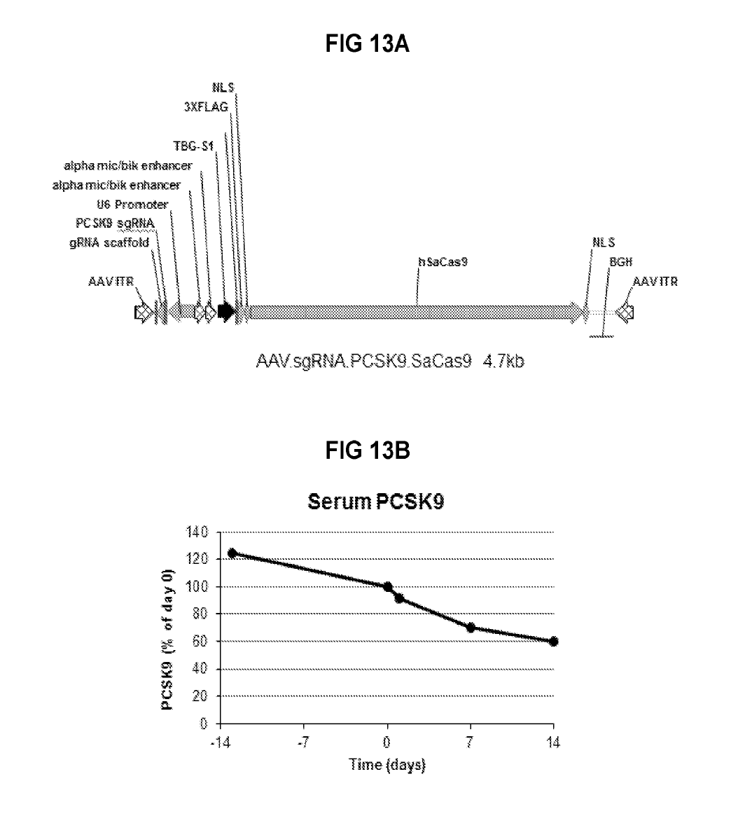

sgRNA3 was cloned into an AAV plasmid contains SaCas9 driven by ABP2.TBG-S1, a liver-specific enhancer/promoter (FIG. 13A). AAV8 vector was produced and intravenously delivered to a rhesus monkey at the dose of 3×10¹³ GC/kg. Two weeks following vector treatment, serum PCSK9 levels were reduced by 40% as compared to day 0 (FIG. 13B). Serum cholesterol, HDL, LDL and triglyceride levels are currently being monitored. DNA and RNA analysis will be performed on liver biopsy samples.

This application contains sequences and a sequence listing, which is hereby incorporated by reference. All publications, patents, and patent applications cited in this application, as well as U.S. Provisional Patent Application No. 62/287,511, filed Jan. 27, 2016, U.S. Provisional Patent Application No. 62/254,225, filed Nov. 12, 2015, U.S. Provisional Patent Application No. 62/183,825, filed Jun. 24, 2015, and U.S. Provisional Patent Application No. 62/153,470, filed Apr. 27, 2015, are hereby incorporated by reference in their entireties as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims.

TABLE 12
(Sequence Listing Free Text)
The following information is provided for sequences containing
free text under numeric identifier <223>.

| SEQ ID NO: | |
|---|---|
| (containing free text) | Free text under <223> |
| 1 | <220> |
| | <221> misc_feature |
| | <222> (4) . . . (23) |
| | <223> sgRNA2 |
| | <220> |
| | <221> misc_feature |
| | <222> (24) . . . (29) |
| | <223> PAM |
| | <220> |
| | <221> misc_feature |
| | <222> (52) . . . (52) |
| | <223> spfash mutation site G->A |
| | <220> |
| | <221> misc_feature |
| | <222> (77) . . . (96) |
| | <223> sgRNA3 |
| | <220> |
| | <221> misc_feature |
| | <222> (91) . . . (96) |
| | <223> PAM (reverse complement sequence) |
| | <220> |
| | <221> misc_feature |
| | <222> (97) . . . (102) |
| | <223> PAM |
| | <220> |
| | <221> misc_feature |
| | <222> (97) . . . (116) |
| | <223> sgRNA1 (reverse complement |
| | sequence) |
| 2 | <220> |
| | <221> misc_feature |
| | <222> (3) . . . (21) |
| | <223> sgRNA1 |
| | <220> |
| | <221> misc_feature |
| | <222> (22) . . . (27) |
| | <223> PAM |
| | <220> |
| | <221> misc_feature |
| | <222> (98) . . . (103) |
| | <223> PAM (reverse complement sequence) |
| | <220> |
| | <221> misc_feature |
| | <222> (102) . . . (104) |
| | <223> W392X mutation:TGG->TGA |
| | <220> |
| | <221> misc_feature |
| | <222> (104) . . . (123) |
| | <223> sgRNA3 (reverse complement |
| | sequence) |
| | <220> |
| | <221> misc_feature |
| | <222> (130) . . . (149) |
| | <223> sgRNA2 |
| | <220> |
| | <221> misc_feature |
| | <222> (150) . . . (155) |
| | <223> PAM |
| 3 | TBG variant TBG-S1 |
| 4 | novel enhancer ABPS2 |
| 5 | target sequence |
| 6 | target sequence |
| 7 | target sequence |
| 8 | target sequence |
| 9 | target sequence |
| 10 | target sequence |
| 12 | spfash G->A mutation sequence |
| 13 | OTC donor sequence (SpCas9, 892-983 bp) |
| 14 | OTC donor sequence (SaCas9, 892-989 bp) |
| 15 | OTC sgRNA1_Fwd |
| 16 | OTC sgRNA1_Rev |
| 17 | OTC sgRNA2_Fwd |
| 18 | OTC sgRNA2_Rev |
| 19 | OTC sgRNA3_Fwd |
| 20 | OTC sgRNA3_Rev |
| 21 | HDR-Fwd |
| 22 | HDR-Rev |
| 23 | OTC_PointM_F |
| 24 | OTC_PointM_R |
| 25 | mOTC hSpCas9 sgRNA1_Fwd |
| 26 | mOTC hSpCas9 sgRNA1_Rev |
| 27 | mOTC hSpCas9 sgRNA2_Fwd |
| 28 | mOTC hSpCas9 sgRNA2_Rev |
| 29 | mOTC hSpCas9 sgRNA3_Fwd |
| 30 | mOTC hSpCas9 sgRNA3_Rev |
| 31 | mOTC hSaCas9 sgRNA1_Fwd |
| 32 | mOTC hSaCas9 sgRNA1_Rev |
| 33 | mOTC hSaCas9 sgRNA2_Fwd |
| 34 | mOTC hSaCas9 sgRNA2_Rev |
| 35 | mOTC hSaCas9 sgRNA3_Fwd |
| 36 | mOTC hSaCas9 sgRNA3_Rev |
| 37 | OTC_OT1F1 |
| 38 | OTC_OT1R1 |
| 39 | OTC_OT1F2 |
| 40 | OTC_OT1R2 |
| 41 | OTC_OT2F1 |
| 42 | OTC_OT2R1 |
| 43 | OTC_OT2F2 |
| 44 | OTC_OT2R2 |
| 45 | OTC_OT3F1 |
| 46 | OTC_OT3R1 |
| 47 | OTC_OT3F2 |
| 48 | OTC_OT3R2 |
| 49 | OTC_OT4F1 |
| 50 | OTC_OT4R1 |
| 51 | OTC_OT4F2 |
| 52 | OTC_OT4R2 |
| 53 | OTC_OT5F1 |
| 54 | OTC_OT5R1 |
| 55 | OTC_OT5F2 |
| 56 | OTC_OT5R2 |
| 57 | OTC_OT6F1 |
| 58 | OTC_OT6R1 |
| 59 | OTC_OT6F2 |
| 60 | OTC_OT6R2 |
| 61 | OTC_OT7F1 |
| 62 | OTC_OT7R1 |
| 63 | OTC_OT7F2 |
| 64 | OTC_OT7R2 |
| 65 | OTC_OT8F1 |
| 66 | OTC_OT8R1 |
| 67 | OTC_OT8F2 |
| 68 | OTC_OT8R2 |
| 69 | OTC_OT9F1 |
| 70 | OTC_OT9R1 |
| 71 | OTC_OT9F2 |
| 72 | OTC_OT9R2 |
| 73 | OTC_OT10F1 |
| 74 | OTC_OT10R1 |
| 75 | OTC_OT10F2 |
| 76 | OTC_OT10R2 |
| 77 | OTC_OT11F1 |
| 78 | OTC_OT11R1 |
| 79 | OTC_OT11F2 |
| 80 | OTC_OT11R2 |
| 81 | OTC_OT12F |
| 82 | OTC_OT12R |
| 83 | OTC_OT13F1 |
| 84 | OTC_OT13R1 |
| 85 | OTC_OT13F2 |
| 86 | OTC_OT13R2 |
| 87 | OTC_OT14F1 |
| 88 | OTC_OT14R1 |
| 89 | OTC_OT14F2 |
| 90 | OTC_OT14R2 |
| 91 | OTC_OT15F1 |
| 92 | OTC_OT15R1 |
| 93 | OTC_OT15F2 |
| 94 | OTC_OT15R2 |
| 95 | OTC_OT16F1 |
| 96 | OTC_OT16R1 |
| 97 | OTC_OT16F2 |
| 98 | OTC_OT16R2 |
| 99 | sgRNA1 |
| 100 | OT1 |
| 101 | OT2 |
| 102 | OT3 |
| 103 | OT4 |
| 104 | OT5 |
| 105 | OT6 |
| 106 | OT7 |
| 107 | OT8 |
| 108 | OT9 |
| 109 | OT10 |
| 110 | OT11 |
| 111 | OT12 |
| 112 | OT13 |
| 113 | OT14 |
| 114 | OT15 |
| 115 | OT16 |
| 116 | OT17 |
| 117 | OT18 |
| 118 | OT19 |
| 119 | OT20 |
| 120 | OT21 |
| 121 | OT22 |
| 122 | OT23 |
| 123 | OT24 |
| 124 | OT25 |
| 125 | OT26 |
| 126 | OT27 |
| 127 | OT28 |
| 128 | OT29 |
| 129 | OT30 |
| 130 | OT31 |
| 131 | OT32 |
| 132 | OT33 |
| 133 | OT34 |
| 134 | OT35 |
| 135 | OT36 |
| 136 | OT37 |
| 137 | OT38 |
| 138 | OT39 |
| 139 | OT40 |
| 140 | OT41 |
| 141 | OT42 |
| 142 | OT43 |
| 143 | OT44 |
| 144 | OT45 |
| 145 | OT46 |
| 146 | OT47 |
| 147 | OT48 |
| 148 | OT49 |
| 149 | dIDUA_sgRNA1_Fwd |
| 150 | dIDUA_sgRNA1_Rev |
| 151 | dIDUA_sgRNA2_Fwd |
| 152 | dlDUA_sgRNA2_Rev |
| 153 | dIDUA_sgRNA3_Fwd |
| 154 | dlDUA_sgRNA3_Rev |
| 155 | dIDUA_sgRNA4_Fwd |
| 156 | dlDUA_sgRNA4_Rev |
| 157 | dIDUA_sgRNA5_Fwd |
| 158 | dlDUA_sgRNA5_Rev |
| 159 | dIDUA_sgRNA6_Fwd |
| 160 | dlDUA_sgRNA6_Rev |
| 161 | hCFTR_sgRNA1_Fwd |
| 162 | hCFTR_sgRNA1_Rev |
| 163 | hCFTR_sgRNA2_Fwd |
| 164 | hCFTR_sgRNA2_Rev |
| 165 | hCFTR_sgRNA3_Fwd |
| 166 | hCFTR_sgRNA3_Rev |
| 167 | hCFTR_sgRNA4_Fwd |
| 168 | hCFTR_sgRNA4_Rev |
| 169 | mFIXsgRNA1_Fwd |
| 170 | mFIXsgRNA1_Rev |
| 171 | mFIXsgRNA2_Fwd |
| 172 | mFIXsgRNA2_Rev |
| 173 | mFIXsgRNA3_Fwd |
| 174 | mFIXsgRNA3_Rev |
| 175 | mFIX_PointM_F |
| 176 | mFIX_PointM_R |
| 177 | rhFIXsgRNA1_Fwd |
| 178 | rhFIXsgRNA1_Rev |
| 179 | rhFIXsgRNA2_Fwd |
| 180 | rhFIXsgRNA2_Rev |
| 181 | rhFIXsgRNA3_Fwd |
| 182 | rhFIXsgRNA3_Rev |
| 183 | EGFP-sgRNAF |
| 184 | EGFP-sgRNAR |
| 185 | PCSK9_sgRNA1F |
| 186 | PCSK9_sgRNA1R |
| 187 | PCSK9_sgRNA2F |
| 188 | PCSK9_sgRNA2R |
| 189 | PCSK9_sgRNA3F |
| 190 | PCSK9_sgRNA3R |
| 191 | PCSK9_sgRNA4F |
| 192 | PCSK9_sgRNA4R |
| 193 | PCSK9_sgRNA5F |
| 194 | PCSK9_sgRNA5R |
| 195 | PCSK9_sgRNA6F |
| 196 | PCSK9_sgRNA6R |
| 197 | PCSK9_sgRNA7F |
| 198 | PCSK9_sgRNA7R |

1. A dual vector system for treating genetic disorders, wherein the system comprises:
(a) a gene editing AAV vector comprising a Cas9 gene under control of regulatory sequences which direct its expression in a target cell comprising a targeted gene which has one or more mutations resulting in a disorder; and
(b) a targeting AAV vector comprising one or more of sgRNAs and a donor template, wherein the sgRNAs comprise at least 20 nucleotides which specifically bind to a selected site in the targeted genes and which is 5′ to a protospacer-adjacent motif (PAM) which is specifically recognized by the Cas9, and wherein the donor template comprises nucleic acid sequences which replaces at least one of the mutations in the targeted gene and;
optionally wherein the ratio of gene editing vector of (a) to (b) is such that (b) is in excess of (a).

2. The dual vector system according to claim 1, wherein the target cells are proliferating cells, progenitor cells, or stem cells.

3. The dual vector system according to claim 2, wherein the proliferating cells are hepatocytes or epithelial cells.

4. The vector system according to claim 1, wherein the ratio of editing vector (a) to targeting vector (b) is about 1:3 to about 1:100, or about 1:10.

5. The vector system according to claim 1, wherein the sgRNA of vector (b) comprises about 24 nucleotides to about 28 nucleotides.

6. The vector system according to claim 1, wherein the donor template is a full-length gene encoding a functioning protein or enzyme.

7. The vector system according to claim 1, wherein the donor template of vector (b) encodes a partial protein or enzyme and is designed to complement a defective gene and to allow production of a functioning protein in the target cell.

8. The vector system according to claim 1, wherein the sgRNA of vector (b) is designed to target an intron of the targeted gene, such that the mutation is corrected by the donor.

9. The vector system according to claim 1, wherein the sgRNA of vector (b) is designed to target a gene, such that the donor template is inserted upstream of the gene defect.

10. The vector system according to claim 1, wherein the vector (b) comprises more than one sgRNA.

11. The vector system according to claim 1, wherein the gene editing vector of (a) and the targeting vector of (b) have the same AAV capsid.

12. The vector system according to claim 11, wherein the AAV capsid is selected from AAV8, AAV9, rh10, AAV6.2, AAV3B, hu37, and/or rh64.

13. The vector system according to claim 1, wherein Cas9 is selected from Staphylococcus aureaus or Staphylococcus pyogenes Cas9.

14. The vector system according to claim 1, wherein the Cas9 is under the control of a tissue-specific promoter.

15. The vector system according to claim 1, wherein the Cas9 is under the control of a constitutive promoter.

16. The vector system according to claim 1, wherein the Cas9 is under the control of a liver-specific promoter.

17. The vector system according to claim 16, wherein the promoter is a human thyroxin-binding globulin (TBG) promoter.

18. A method of treating a disorder in humans by co-administering the vector system according to claim 1.

19. (canceled)

20. A method of treating a liver metabolic disorder in neonates, comprising: co-administering to a subject having a liver metabolic disorder:
(a) a gene editing AAV vector comprising a Cas9 gene under control of regulatory sequences which direct its expression in a hepatocyte comprising a targeted gene which has one or more mutations resulting in a liver metabolic disorder; and
(b) an AAV targeting vector comprising sgRNA and donor template, wherein the sgRNA comprises at least 20 nucleotides which specifically bind to a selected site in the targeted genes and which is 5′ to a protospacer-adjacent motif (PAM) which is specifically recognized by the Cas9, and wherein the donor template comprises nucleic acid sequences which replaces at least one of the mutations in the targeted gene;
wherein the ratio of gene editing AAV vector of (a) to (b) is such that (b) is in excess of (a).

21. The method according to claim 20, wherein the vectors (a) and (b) are delivered essentially simultaneously via the same route.

22. The method according to claim 20, wherein the gene editing vector (a) is suspended in a vehicle for injection at a concentration of about 2×10¹¹ GC/mL to about 2×10¹² GC/mL.

23. The method according to claim 20, wherein the AAV targeting vector (a) is suspended in a vehicle for injection at a concentration of about 2×10¹² GC/mL to about 1×10¹³ GC/mL.

24. The method according to claim 20, wherein the liver metabolic disorder is ornithine transcarbamylase.

25. The method according to claim 24, wherein the sgRNA is targeted to a G/A mutation site in exon4 of the OTC gene, located at nucleotide 243 based on the numbering of the wild-type OTC gene.

26. (canceled)


SEQUENCE LISTING (198 sequences)

SEQ ID NO: 1 - DNA, 120 bp, Mus musculus
  misc_feature (4)..(23): sgRNA2
  misc_feature (24)..(29): PAM
  misc_feature (52)..(52): spfash mutation site G->A
  misc_feature (77)..(96): sgRNA3
  misc_feature (91)..(96): PAM (reverse complement sequence)
  misc_feature (97)..(102): PAM
  misc_feature (97)..(116): sgRNA1 (reverse complement sequence)
ccacacaagacattcacttgggtgtgaatgaaagtctcacagacaccgctcggtttgtaaaacttttcttccttccaaagtttatttcaaactctgatgggttagtttaaaagagaagat

SEQ ID NO: 2 - DNA, 157 bp, Mus musculus
  misc_feature (3)..(21): sgRNA1
  misc_feature (22)..(27): PAM
  misc_feature (98)..(103): PAM (reverse complement sequence)
  misc_feature (102)..(104): W392X mutationTGG->TGA
  misc_feature (104)..(123): sgRNA3 (reverse complement sequence)
  misc_feature (130)..(149): sgRNA2
  misc_feature (150)..(155): PAM
caaatggtgggagctagatattagggtaggaagccagatgctaggtatgagagagccaacagcctcagccctctgcttggcttatagatggagaacaactctgggcagaggtctcaaaggctggggctgtgttggacagcaatcatacagtgggtgt

SEQ ID NO: 3 - DNA, 176 bp, Artificial Sequence
  TBG variant TBG-S1
actcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaga

SEQ ID NO: 4 - DNA, 80 bp, Artificial Sequence
  novel enhancer ABPS2
gttaatttttaaactgtttgctctggttaataatctcaggaggttaatttttaaactgtttgctctggttaataatctca

SEQ ID NO: 5 - DNA, 20 bp, Artificial Sequence
  target sequence
agtttgaaataaactttgga

SEQ ID NO: 6 - DNA, 20 bp, Artificial Sequence
  target sequence
gaaaagttttacaaactgag

SEQ ID NO: 7 - DNA, 20 bp, Artificial Sequence
  target sequence
tcagagtttgaaataaactt

SEQ ID NO: 8 - DNA, 20 bp, Artificial Sequence
  target sequence
tctcttttaaactaacccat

SEQ ID NO: 9 - DNA, 20 bp, Artificial Sequence
  target sequence
cacaagacattcacttgggt

SEQ ID NO: 10 - DNA, 20 bp, Artificial Sequence
  target sequence
aaagtttatttcaaactctg

SEQ ID NO: 11 - DNA, 92 bp, Mus musculus
gaaagtctcacagacaccgctcggtttgtaaaacttttcttccttccaaagtttatttcaaactctgatgggttagtttaaaagagaagatg

SEQ ID NO: 12 - DNA, 92 bp, Artificial Sequence
  spfash G->A mutation sequence
gaaagtctcacagacaccgctcagtttgtaaaacttttcttccttccaaagtttatttcaaactctgatgggttagtttaaaagagaagatg

SEQ ID NO: 13 - DNA, 91 bp, Artificial Sequence
  OTC donor sequence (SpCas9, 892-983 bp)
aaagtctcacagacaccgctcggtttgtaaaacttttcttccttgcaaagtttatttcaaactctgatgggttagtttaaaagagaagatg

SEQ ID NO: 14 - DNA, 98 bp, Artificial Sequence
  OTC donor sequence (SaCas9, 892-989 bp)
gaaagtctcacagacaccgctcggtttgtaaaacttttcttccttccaaagtttaccggtatttcaattggtgatgggttagtttaaaagagaagatg

SEQ ID NO: 15 - DNA, 25 bp, Artificial Sequence
  OTC sgRNA1_Fwd
caccgtctcttttaaactaacccat

SEQ ID NO: 16 - DNA, 25 bp, Artificial Sequence
  OTC sgRNA1_Rev
aaacatgggttagtttaaaagagac

SEQ ID NO: 17 - DNA, 25 bp, Artificial Sequence
  OTC sgRNA2_Fwd
caccgcacaagacattcacttgggt

SEQ ID NO: 18 - DNA, 25 bp, Artificial Sequence
  OTC sgRNA2_Rev
aaacacccaagtgaatgtcttgtgc

SEQ ID NO: 19 - DNA, 25 bp, Artificial Sequence
  OTC sgRNA3_Fwd
caccgaaagtttatttcaaactctg

SEQ ID NO: 20 - DNA, 25 bp, Artificial Sequence
  OTC sgRNA3_Rev
aaaccagagtttgaaataaactttc

SEQ ID NO: 21 - DNA, 21 bp, Artificial Sequence
  HDR-Fwd
tggagcaattctgcacatgga

SEQ ID NO: 22 - DNA, 23 bp, Artificial Sequence
  HDR-Rev
cttactgaacatggcagtttccc

SEQ ID NO: 23 - DNA, 21 bp, Artificial Sequence
  OTC_PointM_F
ggctatgcttgggaatgtcct

SEQ ID NO: 24 - DNA, 22 bp, Artificial Sequence
  OTC_PointM_R
gctacagaatgaaagagaggcg

SEQ ID NO: 25 - DNA, 25 bp, Artificial Sequence
  mOTC hSpCas9 sgRNA1_Fwd
caccgagtttgaaataaactttgga

SEQ ID NO: 26 - DNA, 25 bp, Artificial Sequence
  mOTC hSpCas9 sgRNA1_Rev
aaactccaaagtttatttcaaactc

SEQ ID NO: 27 - DNA, 24 bp, Artificial Sequence
  mOTC hSpCas9 sgRNA2_Fwd
caccgaaaagttttacaaactgag

SEQ ID NO: 28 - DNA, 24 bp, Artificial Sequence
  mOTC hSpCas9 sgRNA2_Rev
aaacctcagtttgtaaaacttttc

SEQ ID NO: 29 - DNA, 25 bp, Artificial Sequence
  mOTC hSpCas9 sgRNA3_Fwd
caccgtcagagtttgaaataaactt

SEQ ID NO: 30 - DNA, 25 bp, Artificial Sequence
  mOTC hSpCas9 sgRNA3_Rev
aaacaagtttatttcaaactctgac

SEQ ID NO: 31 - DNA, 25 bp, Artificial Sequence
  mOTC hSaCas9 sgRNA1_Fwd
caccgtctcttttaaactaacccat

SEQ ID NO: 32 - DNA, 25 bp, Artificial Sequence
  mOTC hSaCas9 sgRNA1_Rev
aaacatgggttagtttaaaagagac

SEQ ID NO: 33 - DNA, 25 bp, Artificial Sequence
  mOTC hSaCas9 sgRNA2_Fwd
caccgcacaagacattcacttgggt

SEQ ID NO: 34 - DNA, 25 bp, Artificial Sequence
  mOTC hSaCas9 sgRNA2_Rev
aaacacccaagtgaatgtcttgtgc

SEQ ID NO: 35 - DNA, 25 bp, Artificial Sequence
  mOTC hSaCas9 sgRNA3_Fwd
caccgaaagtttatttcaaactctg

SEQ ID NO: 36 - DNA, 25 bp, Artificial Sequence
  mOTC hSaCas9 sgRNA3_Rev
aaaccagagtttgaaataaactttc

SEQ ID NO: 37 - DNA, 21 bp, Artificial Sequence
  OTC_OT1F1
ctggtgcctttttctatcgcc

SEQ ID NO: 38 - DNA, 22 bp, Artificial Sequence
  OTC_OT1R1
ccaagagcaactacaatggctt

SEQ ID NO: 39 - DNA, 20 bp, Artificial Sequence
  OTC_OT1F2
gcattttcatgagcattcca

SEQ ID NO: 40 - DNA, 20 bp, Artificial Sequence
  OTC_OT1R2
catgttgtgcctgcatctct

SEQ ID NO: 41 - DNA, 21 bp, Artificial Sequence
  OTC_OT2F1
gcagactccaagatgcaagac

SEQ ID NO: 42 - DNA, 21 bp, Artificial Sequence
  OTC_OT2R1
gatgttgttccacccgcatct

SEQ ID NO: 43 - DNA, 20 bp, Artificial Sequence
  OTC_OT2F2
cactgagccaagtcactgga

SEQ ID NO: 44 - DNA, 20 bp, Artificial Sequence
  OTC_OT2R2
agggacaaaaccaaacagca

SEQ ID NO: 45 - DNA, 20 bp, Artificial Sequence
  OTC_OT3F1
tggccttctaaagcaaccaa

SEQ ID NO: 46 - DNA, 21 bp, Artificial Sequence
  OTC_OT3R1
ccgtctcccagatcacatgac

SEQ ID NO: 47 - DNA, 27 bp, Artificial Sequence
  OTC_OT3F2
ataactcataatctatgcatggcacaa

SEQ ID NO: 48 - DNA, 24 bp, Artificial Sequence
  OTC_OT3R2
tttgatcatggtgtttatcagagc

SEQ ID NO: 49 - DNA, 20 bp, Artificial Sequence
  OTC_OT4F1
gtccccgacaaaccaagcta

SEQ ID NO: 50 - DNA, 20 bp, Artificial Sequence
  OTC_OT4R1
tgaactggcagtatgcaggg

SEQ ID NO: 51 - DNA, 20 bp, Artificial Sequence
  OTC_OT4F2
aacatggtttctgccctcag

SEQ ID NO: 52 - DNA, 20 bp, Artificial Sequence
  OTC_OT4R2
ggaccatgccgaactcttac

SEQ ID NO: 53 - DNA, 20 bp, Artificial Sequence
  OTC_OT5F1
ttgagacctagctcatgccc

SEQ ID NO: 54 - DNA, 20 bp, Artificial Sequence
  OTC_OT5R1
taacgcagaactggcacagg

SEQ ID NO: 55 - DNA, 20 bp, Artificial Sequence
  OTC_OT5F2
ctctcccatggagaaagctg

SEQ ID NO: 56 - DNA, 20 bp, Artificial Sequence
  OTC_OT5R2
gaatgtggcattggcttttt

SEQ ID NO: 57 - DNA, 20 bp, Artificial Sequence
  OTC_OT6F1
gagagagccaatctgcccat

SEQ ID NO: 58 - DNA, 20 bp, Artificial Sequence
  OTC_OT6R1
caccggaaacgtgtgagaga

SEQ ID NO: 59 - DNA, 20 bp, Artificial Sequence
  OTC_OT6F2
acttcccatgatcccattga

SEQ ID NO: 60 - DNA, 20 bp, Artificial Sequence
  OTC_OT6R2
agcttccctccaagtgtcct

SEQ ID NO: 61 - DNA, 21 bp, Artificial Sequence
  OTC_OT7F1
gatgggcataagcccgaagta

SEQ ID NO: 62 - DNA, 21 bp, Artificial Sequence
  OTC_OT7R1
taaggcccagtgttgttgtgt

SEQ ID NO: 63 - DNA, 20 bp, Artificial Sequence
  OTC_OT7F2
gtagcaggggctctgtgaag

SEQ ID NO: 64 - DNA, 20 bp, Artificial Sequence
  OTC_OT7R2
tggcctgaaatacccagaac

SEQ ID NO: 65 - DNA, 20 bp, Artificial Sequence
  OTC_OT8F1
gttgaattcgcgtgtccagg

SEQ ID NO: 66 - DNA, 20 bp, Artificial Sequence
  OTC_OT8R1
tcccatggcgagaatgtcac

SEQ ID NO: 67 - DNA, 21 bp, Artificial Sequence
  OTC_OT8F2
ccctgtaggaaacacagagga

SEQ ID NO: 68 - DNA, 22 bp, Artificial Sequence
  OTC_OT8R2
tgctttggatgttgattctaaa

SEQ ID NO: 69 - DNA, 20 bp, Artificial Sequence
  OTC_OT9F1
ggcaaaggactagcttgcac

SEQ ID NO: 70 - DNA, 20 bp, Artificial Sequence
  OTC_OT9R1
gggtgctatgaggaccagtg

SEQ ID NO: 71 - DNA, 20 bp, Artificial Sequence
  OTC_OT9F2
cagtggtgtgtggagagctg

SEQ ID NO: 72 - DNA, 21 bp, Artificial Sequence
  OTC_OT9R2
agagagagcgcttgactttga

SEQ ID NO: 73 - DNA, 20 bp, Artificial Sequence
  OTC_OT10F1
ctttgactcccggcgaaaga

SEQ ID NO: 74 - DNA, 20 bp, Artificial Sequence
  OTC_OT10R1
ttgtccatccgggtcattgc

SEQ ID NO: 75 - DNA, 20 bp, Artificial Sequence
  OTC_OT10F2
aagtccttcttgcccaactt

SEQ ID NO: 76 - DNA, 18 bp, Artificial Sequence
  OTC_OT10R2
cagccccaatgcattttt

SEQ ID NO: 77 - DNA, 20 bp, Artificial Sequence
  OTC_OT11F1
attacaggtcctggttgggc

SEQ ID NO: 78 - DNA, 20 bp, Artificial Sequence
  OTC_OT11R1
actgagcctggtagagcctt

SEQ ID NO: 79 - DNA, 20 bp, Artificial Sequence
  OTC_OT11F2
ggaaggtgaaggaaggaagg

SEQ ID NO: 80 - DNA, 24 bp, Artificial Sequence
  OTC_OT11R2
ttttctaggaattcaggacataca

SEQ ID NO: 81 - DNA, 22 bp, Artificial Sequence
  OTC_OT12F
tcatggtccttaaaatttttgc

SEQ ID NO: 82 - DNA, 20 bp, Artificial Sequence
  OTC_OT12R
tccaggtatgcaaagtggat

SEQ ID NO: 83 - DNA, 25 bp, Artificial Sequence
  OTC_OT13F1
ttcagttgtactttggatgctctga

SEQ ID NO: 84 - DNA, 20 bp, Artificial Sequence
  OTC_OT13R1
catctgaatagcagcaggcg

SEQ ID NO: 85 - DNA, 20 bp, Artificial Sequence
  OTC_OT13F2
tagcacagcccaaatgactt

SEQ ID NO: 86 - DNA, 22 bp, Artificial Sequence
  OTC_OT13R2
tcatgaaaccccataatcagaa

SEQ ID NO: 87 - DNA, 22 bp, Artificial Sequence
  OTC_OT14F1
agtgggtcatcctttgttaccc

SEQ ID NO: 88 - DNA, 20 bp, Artificial Sequence
  OTC_OT14R1
tgccagttatcagccaagca

SEQ ID NO: 89 - DNA, 21 bp, Artificial Sequence
  OTC_OT14F2
cccaggaacttaactcaggtg

SEQ ID NO: 90 - DNA, 22 bp, Artificial Sequence
  OTC_OT14R2
tgccatttgacctcataagtct

SEQ ID NO: 91 - DNA, 21 bp, Artificial Sequence
  OTC_OT15F1
ttcagcccccttgagtgttta

SEQ ID NO: 92 - DNA, 22 bp, Artificial Sequence
  OTC_OT15R1
gtctctgagcacaaagagacga

SEQ ID NO: 93 - DNA, 20 bp, Artificial Sequence
  OTC_OT15F2
ttgcctgtcccaactagagc

SEQ ID NO: 94 - DNA, 20 bp, Artificial Sequence
  OTC_OT15R2
ggcccaagaatgcacattta

SEQ ID NO: 95 - DNA, 20 bp, Artificial Sequence
  OTC_OT16F1
ccacacactggctaggactg

SEQ ID NO: 96 - DNA, 20 bp, Artificial Sequence
  OTC_OT16R1
actggcagcacttgagacaa

SEQ ID NO: 97 - DNA, 20 bp, Artificial Sequence
  OTC_OT16F2
gatggcatgctgtggttttt

SEQ ID NO: 98 - DNA, 20 bp, Artificial Sequence
  OTC_OT16R2
caatgcttccacacagaacc

SEQ ID NO: 99 - DNA, 20 bp, Artificial Sequence
  sgRNA1
tctcttttaaactaacccat

SEQ ID NO: 100 - DNA, 20 bp, Artificial Sequence
  OT1
tatcttttcaactaacccaa

SEQ ID NO: 101 - DNA, 20 bp, Artificial Sequence
  OT2
tttcttttatactagcccat

SEQ ID NO: 102 - DNA, 20 bp, Artificial Sequence
  OT3
tctttttttaaataacccat

SEQ ID NO: 103 - DNA, 20 bp, Artificial Sequence
  OT4
tggcatttaaactaacccaa

SEQ ID NO: 104 - DNA, 20 bp, Artificial Sequence
  OT5
tcttcatgaaactaacccat

SEQ ID NO: 105 - DNA, 20 bp, Artificial Sequence
  OT6
atttttttaaacaaacccat

SEQ ID NO: 106 - DNA, 20 bp, Artificial Sequence
  OT7
gatcttgtaatctaacccat

SEQ ID NO: 107 - DNA, 20 bp, Artificial Sequence
  OT8
ccttttataatctaacccat

SEQ ID NO: 108 - DNA, 20 bp, Artificial Sequence
  OT9
tctgttttaaagtaaccctt

SEQ ID NO: 109 - DNA, 20 bp, Artificial Sequence
  OT10
ttgcttttcaactaacccaa

SEQ ID NO: 110 - DNA, 20 bp, Artificial Sequence
  OT11
aatattttaaactatcccat

SEQ ID NO: 111 - DNA, 20 bp, Artificial Sequence
  OT12
ttactttaaaactatcccat

SEQ ID NO: 112 - DNA, 20 bp, Artificial Sequence
  OT13
acacttttaaaataacccag

SEQ ID NO: 113 - DNA, 20 bp, Artificial Sequence
  OT14
aatcttctaaaccaacccat

SEQ ID NO: 114 - DNA, 20 bp, Artificial Sequence
  OT15
tctttattagactaacccag

SEQ ID NO: 115 - DNA, 20 bp, Artificial Sequence
  OT16
tttcatttaacataacccat

SEQ ID NO: 116 - DNA, 20 bp, Artificial Sequence
  OT17
tctttttagaagtaacccat

SEQ ID NO: 117 - DNA, 20 bp, Artificial Sequence
  OT18
tccctttttcactaacccac

SEQ ID NO: 118 - DNA, 20 bp, Artificial Sequence
  OT19
tctttgtaaaattaacccat

SEQ ID NO: 119 - DNA, 20 bp, Artificial Sequence
  OT20
tttattttaaagtaacccac

SEQ ID NO: 120 - DNA, 20 bp, Artificial Sequence
  OT21
tatctttcaaaataacccac

SEQ ID NO: 121 - DNA, 20 bp, Artificial Sequence
  OT22
tatcttggaaactaacccct

SEQ ID NO: 122 - DNA, 20 bp, Artificial Sequence
  OT23
cttcttttaaacaaacccag

SEQ ID NO: 123 - DNA, 20 bp, Artificial Sequence
  OT24
aatcttttatactaaccaat

SEQ ID NO: 124 - DNA, 20 bp, Artificial Sequence
  OT25
tattttttaaattaactcat

SEQ ID NO: 125 - DNA, 20 bp, Artificial Sequence
  OT26
tttattttaaaataacacat

SEQ ID NO: 126 - DNA, 20 bp, Artificial Sequence
  OT27
tctctttcaaactatcccaa

SEQ ID NO: 127 - DNA, 20 bp, Artificial Sequence
  OT28
tatctttaaaaataacacat

SEQ ID NO: 128 - DNA, 20 bp, Artificial Sequence
  OT29
tttctttaaaaataacacat

SEQ ID NO: 129 - DNA, 20 bp, Artificial Sequence
  OT30
gttcttttaaaaaaacccat

SEQ ID NO: 130 - DNA, 20 bp, Artificial Sequence
  OT31
tctgatttaaaataacacat

SEQ ID NO: 131 - DNA, 20 bp, Artificial Sequence
  OT32
cctcttataacctaacccac

SEQ ID NO: 132 - DNA, 20 bp, Artificial Sequence
  OT33
cctcttgtaagctaacccac

SEQ ID NO: 133 - DNA, 20 bp, Artificial Sequence
  OT34
cctattttaaagtaaccctt

SEQ ID NO: 134 - DNA, 20 bp, Artificial Sequence
  OT35
catcttgtaaactagcccat

SEQ ID NO: 135 - DNA, 20 bp, Artificial Sequence
  OT36
actcttttacaataacacat

SEQ ID NO: 136 - DNA, 20 bp, Artificial Sequence
  OT37
tctcctttagcctaactcat

SEQ ID NO: 137 - DNA, 20 bp, Artificial Sequence
  OT38
cctctttaaaaataacccct

SEQ ID NO: 138 - DNA, 20 bp, Artificial Sequence
  OT39
tatctttgaaataaacccat

SEQ ID NO: 139 - DNA, 20 bp, Artificial Sequence
  OT40
tatatttaaaactaagccat

SEQ ID NO: 140 - DNA, 20 bp, Artificial Sequence
  OT41
tgactttaaaactaagccat

SEQ ID NO: 141 - DNA, 20 bp, Artificial Sequence
  OT42
cttcttttcaactatcccat

SEQ ID NO: 142 - DNA, 20 bp, Artificial Sequence
  OT43
gctcttataaattaacccag

SEQ ID NO: 143 - DNA, 20 bp, Artificial Sequence
  OT44
tttcttttaaattaagccat

SEQ ID NO: 144 - DNA, 20 bp, Artificial Sequence
  OT45
tctctttgtcacaaacccat

SEQ ID NO: 145 - DNA, 20 bp, Artificial Sequence
  OT46
tttcttctaagttaacccat

SEQ ID NO: 146 - DNA, 20 bp, Artificial Sequence
  OT47
tcacttccaaactaccccat

SEQ ID NO: 147 - DNA, 20 bp, Artificial Sequence
  OT48
tatcttataaaataacccag

SEQ ID NO: 148 - DNA, 20 bp, Artificial Sequence
  OT49
tcaatttttaactatcccat

SEQ ID NO: 149 - DNA, 25 bp, Artificial Sequence
  dIDUA_sgRNA1_Fwd
caccgttctgatgagggctccgcgg

SEQ ID NO: 150 - DNA, 25 bp, Artificial Sequence
  dIDUA_sgRNA1_Rev
aaacccgcggagccctcatcagaac

SEQ ID NO: 151 - DNA, 24 bp, Artificial Sequence
  dIDUA_sgRNA2_Fwd
caccgcacctggtgctcgtggacg

SEQ ID NO: 152 - DNA, 24 bp, Artificial Sequence
  dlDUA_sgRNA2_Rev
aaaccgtccacgagcaccaggtgc

SEQ ID NO: 153 - DNA, 24 bp, Artificial Sequence
  dIDUA_sgRNA3_Fwd
caccgggccgcgtccacgagcacc

SEQ ID NO: 154 - DNA, 24 bp, Artificial Sequence
  dlDUA_sgRNA3_Rev
aaacggtgctcgtggacgcggccc

SEQ ID NO: 155 - DNA, 24 bp, Artificial Sequence
  dIDUA_sgRNA4_Fwd
caccgtccacgagcaccaggtgcg

SEQ ID NO: 156 - DNA, 24 bp, Artificial Sequence
  dlDUA_sgRNA4_Rev
aaaccgcacctggtgctcgtggac

SEQ ID NO: 157 - DNA, 25 bp, Artificial Sequence
  dIDUA_sgRNA5_Fwd
caccgtggacgcggcccgcgcgctg

SEQ ID NO: 158 - DNA, 25 bp, Artificial Sequence
  dlDUA_sgRNA5_Rev
aaaccagcgcgcgggccgcgtccac

SEQ ID NO: 159 - DNA, 24 bp, Artificial Sequence
  dIDUA_sgRNA6_Fwd
caccgcttctgatgagggctccgc

SEQ ID NO: 160 - DNA, 24 bp, Artificial Sequence
  dlDUA_sgRNA6_Rev
aaacgcggagccctcatcagaagc

SEQ ID NO: 161 - DNA, 25 bp, Artificial Sequence
  hCFTR_sgRNA1_Fwd
caccgaatggtgccaggcataatcc

SEQ ID NO: 162 - DNA, 25 bp, Artificial Sequence
  hCFTR_sgRNA1_Rev
aaacggattatgcctggcaccattc

SEQ ID NO: 163 - DNA, 25 bp, Artificial Sequence
  hCFTR_sgRNA2_Fwd
caccgcaaagcatgccaactagaag

SEQ ID NO: 164 - DNA, 25 bp, Artificial Sequence
  hCFTR_sgRNA2_Rev
aaaccttctagttggcatgctttgc

SEQ ID NO: 165 - DNA, 25 bp, Artificial Sequence
  hCFTR_sgRNA3_Fwd
caccgaccattaaagaaaatatcat

SEQ ID NO: 166 - DNA, 25 bp, Artificial Sequence
  hCFTR_sgRNA3_Rev
aaacatgatattttctttaatggtc

SEQ ID NO: 167 - DNA, 25 bp, Artificial Sequence
  hCFTR_sgRNA4_Fwd
caccgagtttcttacctcttctagt

SEQ ID NO: 168 - DNA, 25 bp, Artificial Sequence
  hCFTR_sgRNA4_Rev
aaacactagaagaggtaagaaactc

SEQ ID NO: 169 - DNA, 25 bp, Artificial Sequence
  mFIXsgRNA1_Fwd
caccgtttaggatatctactcagta

SEQ ID NO: 170 - DNA, 25 bp, Artificial Sequence
  mFIXsgRNA1_Rev
aaactactgagtagatatcctaaac

SEQ ID NO: 171 - DNA, 25 bp, Artificial Sequence
  mFIXsgRNA2_Fwd
caccgacaaacctgcacattcggta

SEQ ID NO: 172 - DNA, 25 bp, Artificial Sequence
  mFIXsgRNA2_Rev
aaactaccgaatgtgcaggtttgtc

SEQ ID NO: 173 - DNA, 24 bp, Artificial Sequence
  mFIXsgRNA3_Fwd
caccgcacctgaacaccgtcatgg

SEQ ID NO: 174 - DNA, 24 bp, Artificial Sequence
  mFIXsgRNA3_Rev
aaacccatgacggtgttcaggtgc

SEQ ID NO: 175 - DNA, 20 bp, Artificial Sequence
  mFIX_PointM_F
cgagggagatggacaacaat

SEQ ID NO: 176 - DNA, 20 bp, Artificial Sequence
  mFIX_PointM_R
gcaccacaagccctgtaaat

SEQ ID NO: 177 - DNA, 24 bp, Artificial Sequence
  rhFIXsgRNA1_Fwd
caccgcgtgtgaacatgatcatgg

SEQ ID NO: 178 - DNA, 24 bp, Artificial Sequence
  rhFIXsgRNA1_Rev
aaacccatgatcatgttcacacgc

SEQ ID NO: 179 - DNA, 25 bp, Artificial Sequence
  rhFIXsgRNA2_Fwd
caccgtttaggatatctactcagtg

SEQ ID NO: 180 - DNA, 25 bp, Artificial Sequence
  rhFIXsgRNA2_Rev
aaaccactgagtagatatcctaaac

SEQ ID NO: 181 - DNA, 25 bp, Artificial Sequence
  rhFIXsgRNA3_Fwd
caccgacaaacctgtacattcagca

SEQ ID NO: 182 - DNA, 25 bp, Artificial Sequence
  rhFIXsgRNA3_Rev
aaactgctgaatgtacaggtttgtc

SEQ ID NO: 183 - DNA, 24 bp, Artificial Sequence
  EGFP-sgRNAF
caccgtcgtgctgcttcatgtggt

SEQ ID NO: 184 - DNA, 24 bp, Artificial Sequence
  EGFP-sgRNAR
aaacaccacatgaagcagcacgac

SEQ ID NO: 185 - DNA, 24 bp, Artificial Sequence
  PCSK9_sgRNA1F
caccgatgctctgggcaaagacag

SEQ ID NO: 186 - DNA, 24 bp, Artificial Sequence
  PCSK9_sgRNA1R
aaacctgtctttgcccagagcatc

SEQ ID NO: 187 - DNA, 25 bp, Artificial Sequence
  PCSK9_sgRNA2F
caccgatctcctagacaccagcata

SEQ ID NO: 188 - DNA, 25 bp, Artificial Sequence
  PCSK9_sgRNA2R
aaactatgctggtgtctaggagatc

SEQ ID NO: 189 - DNA, 25 bp, Artificial Sequence
  PCSK9_sgRNA3F
caccgaagttggtccccaaagtccc

SEQ ID NO: 190 - DNA, 25 bp, Artificial Sequence
  PCSK9_sgRNA3R
aaacgggactttggggaccaacttc

SEQ ID NO: 191 - DNA, 24 bp, Artificial Sequence
  PCSK9_sgRNA4F
caccgcagcacctgctttgtgtca

SEQ ID NO: 192 - DNA, 24 bp, Artificial Sequence
  PCSK9_sgRNA4R
aaactgacacaaagcaggtgctgc

SEQ ID NO: 193 - DNA, 25 bp, Artificial Sequence
  PCSK9_sgRNA5F
caccgagtctctgcctcaactcggc

SEQ ID NO: 194 - DNA, 25 bp, Artificial Sequence
  PCSK9_sgRNA5R
aaacgccgagttgaggcagagactc

SEQ ID NO: 195 - DNA, 25 bp, Artificial Sequence
  PCSK9_sgRNA6F
caccgatgacatctttggcagagaa

SEQ ID NO: 196 - DNA, 25 bp, Artificial Sequence
  PCSK9_sgRNA6R
aaacttctctgccaaagatgtcatc

SEQ ID NO: 197 - DNA, 24 bp, Artificial Sequence
  PCSK9_sgRNA7F
caccgcctggttccctgaggacca

SEQ ID NO: 198 - DNA, 24 bp, Artificial Sequence
  PCSK9_sgRNA7R
aaactggtcctcagggaaccaggc